(19) United States
(12) Patent Application Publication
(10) Pub. No.: US 2024/0366629 A1
(43) Pub. Date: Nov. 7, 2024

(54) Porphyrin Complexes as Antidotes for Carbon Monoxide Exposure and Methods of Use for Same

(71) Applicant: The Regents of the University of California, Oakland, CA (US)

(72) Inventors: Timothy C. Johnstone, Santa Cruz, CA (US); Daniel G. Droege, Santa Cruz, CA (US)

(21) Appl. No.: 18/683,744

(22) PCT Filed: Aug. 31, 2022
(86) PCT No.: PCT/US2022/042252

§ 371 Date: Feb. 14, 2024

Related U.S. Application Data
(60) Provisional application No. 63/240,294, filed on Sep. 2, 2021.
(60) Provisional application No. 63/392,733, filed on Jul. 27, 2022.

(51) Int. Cl. A61K 31/555; C07D 487/22; C07F 15/02

(52) U.S. Cl. CPC A61K 31/555; C07D 487/22; C07F 15/025

(57) ABSTRACT

Compounds for binding carbon monoxide are provided (e.g., to sequest carbon monoxide in a composition). Compounds according to certain embodiments include water soluble metal porphyrin complexes having substituents that provide for water solubility at physiological pH and form a hydrophobic carbon monoxide (CO)-binding pocket. In certain embodiments. metal porphyrin complexes exhibit limited cellular uptake. In some instances, the compounds described herein are capable of rescuing CO-poisoned red blood cells. Methods for treating a subject exposed to carbon monoxide (e.g., experiencing carbon monoxide poisoning) are also provided. Compositions for practicing the subject methods are also described.

### CROSS REFERENCE TO RELATED APPLICATIONS

Pursuant to 35 U.S.C. § 119 (e), this application claims priority to the filing date of U.S. Provisional Patent Application Ser. No. 63/240,294 filed on Sep. 2, 2021 and U.S. Provisional Patent Application Ser. No. 63/392,733 filed on Jul. 27, 2022; the disclosures of which applications are incorporated herein by reference in its entirety.

### INTRODUCTION

Carbon monoxide (CO) has a complex biological chemistry. It is celebrated as an endogenous gasotransmitter, and low doses of CO have demonstrated salutary effects in health conditions ranging from cancer to coronary heart disease. Nevertheless, high levels of CO exposure are harmful and can ultimately lead to death. A portion of the toxicity of CO is attributed to its ability to bind hemoglobin (Hb), forming carboxyhemoglobin (COHb) and in-hibiting oxygen transport. Hemoglobin binds CO approximately 200-250-fold more strongly than oxygen (O₂), and the distinctively red-shifted Soret band of COHb is used clinically to assess CO exposure. Elevated COHb levels have been associated with negative outcomes. CO poisoning is the most common form of poisoning worldwide. In the United States alone, over 50,000 emergency department visits each year are attributed to CO exposure. Despite the prevalence of CO poisoning, there is no clinically-approved anti-dote available. Current best practices involve placing the afflicted subject in fresh air, delivering 100% O₂, or administering superatmospheric levels of O₂ in a hyperbaric chamber. These treatments all serve to clear CO from the body by displacing it from metalloproteins with O₂. The typical half-life of COHb in the bloodstream is 5.3 h, but hyperbaric O₂ (1.5-3 atm) can decrease this half-life to <1 h. Unfortunately, these large chambers are generally located in tertiary care centers to which patients must be transported. Moreover, hospitals typically house only a few such chambers, which would be rapidly overwhelmed in the event of a mass exposure.

### SUMMARY

Compounds for binding carbon monoxide are provided (e.g., to sequester carbon monoxide in a composition). Compounds according to certain embodiments include water soluble metal porphyrin complexes having substituents that provide for water solubility at physiological pH and form a hydrophobic carbon monoxide (CO)-binding pocket. In certain embodiments, metal porphyrin complexes exhibit limited cellular uptake. In some instances, the compounds described herein are capable of rescuing CO-poisoned red blood cells. Methods for treating a subject exposed to carbon monoxide (e.g., experiencing carbon monoxide poisoning) are also provided. Compositions for practicing the subject methods are also described.

In some embodiments, compounds of interest include a compound of formula (I):

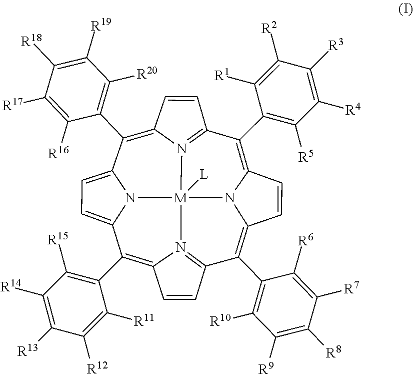

wherein:

R², R⁴, R⁷, R⁹, R¹², R¹⁴, R¹⁷ and R¹⁹ are each independently selected from hydrogen, hydroxy, alkoxy, amine, cyano, thiol, halogen, alkyl, substituted alkyl, haloalkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;

R¹, R⁵, R⁶, R¹⁰, R¹¹, R¹⁵, R¹⁶ and R²⁰ are each independently selected from substituted alkyl, haloalkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;

R³, R⁸, R¹³ and R¹⁸ are each independently a water soluble group;

M is a metal; and

L is a ligand,

or a salt, solvate or hydrate thereof.

In some embodiments, R² is hydrogen. In some embodiments, R⁴ is hydrogen. In some embodiments, R⁷ is hydrogen. In some embodiments, R⁹ is hydrogen. In some embodiments, R¹² is hydrogen. In some embodiments, R¹⁴ is hydrogen. In some embodiments, R¹⁷ is hydrogen. In some embodiments, R¹⁹ is hydrogen. In some embodiments, R², R⁴, R⁷, R⁹, R¹², R¹⁴, R¹⁷ and R¹⁹ are each independently alkyl, such as an alkyl selected from methyl, ethyl, n-propyl, isopropyl, n-butyl, t-butyl, pentyl and hexyl.

In some embodiments, R¹ is an aryl group. In some instances, R¹ is selected from the group consisting of phenyl, 4-methylphenyl, 4-propylphenyl, 4-tertbutylphenyl, 3,5-dimethylphenyl, and 3,5-dichlorophenyl. In some instances, R¹ is phenyl. In some instances, R⁵ is selected from the group consisting of phenyl, 4-methylphenyl, 4-propylphenyl, 4-tertbutylphenyl, 3,5-dimethylphenyl, and 3,5-dichlorophenyl. In some embodiments, R⁵ is an aryl group. In some instances, R⁵ is phenyl. In some embodiments, R⁶ is an aryl group. In some instances, R⁶ is selected from the group consisting of phenyl, 4-methylphenyl, 4-propylphenyl, 4-tertbutylphenyl, 3,5-dimethylphenyl, and 3,5-dichlorophenyl. In some instances, R⁶ is phenyl. In some embodiments, R¹⁰ is an aryl group. In some instances, R¹⁰ is selected from the group consisting of phenyl, 4-methylphenyl, 4-propylphenyl, 4-tertbutylphenyl, 3,5-dimethylphenyl, and 3,5-dichlorophenyl. In some instances, R¹⁰ is phenyl. In some embodiments, R¹¹ is an aryl group. In some instances, R¹¹ is selected from the group consisting of phenyl, 4-methylphenyl, 4-propylphenyl, 4-tertbutylphenyl, 3,5-dimethylphenyl, and 3,5-dichlorophenyl. In some instances, R¹¹ is phenyl. In some embodiments, R¹⁵ is an aryl group. In some instances, R¹⁵ is selected from the group consisting of phenyl, 4-methylphenyl, 4-propylphenyl, 4-tertbutylphenyl, 3,5-dimethylphenyl, and 3,5-dichlorophenyl. In some instances, R¹⁵ is phenyl. In some embodiments, R¹⁶ is an aryl group. In some instances, R¹⁶ is selected from the group consisting of phenyl, 4-methylphenyl, 4-propylphenyl, 4-tertbutylphenyl, 3,5-dimethylphenyl, and 3,5-dichlorophenyl. In some instances, R¹⁶ is phenyl. In some embodiments, R²⁰ is an aryl group. In some instances, R²⁰ is selected from the group consisting of phenyl, 4-methylphenyl, 4-propylphenyl, 4-tertbutylphenyl, 3,5-dimethylphenyl, and 3,5-dichlorophenyl. In some instances, R²⁰ is phenyl.

In some embodiments, R³, R⁸, R¹³ and R¹⁸ are each a group that increase the water solubility of the compound. In some instances, R³, R⁸, R¹³ and R¹⁸ are each a cationic group. In some instances, R³, R⁸, R¹³ and R¹⁸ are each an anionic group. In some instances, R³, R⁸, R¹³ and R¹⁸ are each a zwitterionic group. In certain instances, R³, R⁸, R¹³ and R¹⁸ are each sulfonate.

In some embodiments, M is a metal selected from the group consisting of Ti, Cr, Fe, Co, Ni, Cu, Zn, Mn, Sn, Pb, Mo, Ru, Rh, Pd, Cd, Pt, Ir, and Os. In some embodiments, M in the metal porphyrin complexes is Fe. In some instances, M is Ti. In some instances, M is Cr. In some instances, M is Co. In some instances, M is Ni. In some instances, M is Cu. In some instances, M is Zn. In some instances, M is Mn. In some instances, M is Sn. In some instances, M is Pb. In some instances, M is Mo. In some instances, M is Ru. In some instances, M is Rh.

In some instances, M is Pd. In some instances, M is Cd. In some instances, M is Pt. In some instances, M is Ir. In some instances, M is Os.

In some embodiments, L is hydroxy.

In some embodiments, the compound is a salt. In certain instances, the compound is a tetrasodium salt. In certain instances, the compound is sodium 5,10,15,20-tetrakis (2,6-diphenyl-4-(sulfonate)phenyl)porphyrinatohydroxoiron(III):

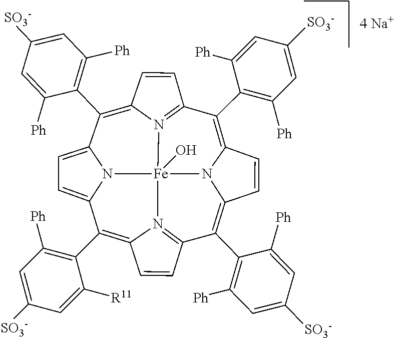

In certain embodiments, the compound is formed from contacting sodium 5,10,15,20-tetrakis (2,6-diphenyl-4-(sulfonate)phenyl)porphyrinatohydroxoiron(III) with a reducing agent. In some embodiments, the reducing agent contacted with the compound is sodium dithionite to form a compound of formula (II):

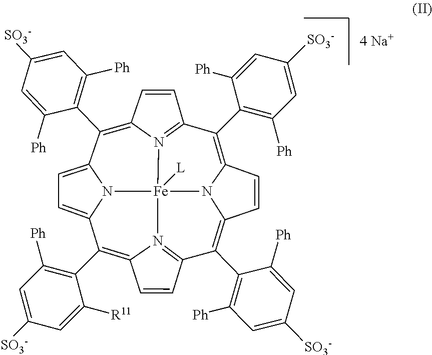

Aspects of the disclosure also include methods for binding carbon monoxide with one or more of the compounds described herein. In practicing the subject methods, one or more of the water soluble metal porphyrin complexes is contacted with a composition containing carbon monoxide. In some instances, contacting the composition with the water soluble metal porphyrin complexes is sufficient to bind 50% or more of the carbon monoxide in the composition, such as 75% or more and including 95% or more of the carbon monoxide in the composition. In some embodiments, the composition contacted with the water soluble metal porphyrin complexes is a biological fluid. In certain instances, the composition includes red blood cells. In certain cases, the composition is whole blood. In some embodiments, the water soluble metal porphyrin complexes are capable of binding carbon monoxide with limited-to-no cellular uptake.

In some embodiments, methods include treating a subject exposed to carbon monoxide, such as a subject experiencing carbon monoxide poisoning. In some instances, the compounds described herein are capable of rescuing CO-poisoned red blood cells in a subject in vivo. In other instances, water soluble metal porphyrin complexes are capable of rescuing CO-poisoned red blood cells in a subject ex vivo (e.g., using an extracorporeal blood treatment system).

### BRIEF DESCRIPTION OF THE FIGURES

FIG. 1 depicts the ¹H NMR spectrum (500 MHZ, DMSO-d₆) of Compound 6.

FIG. 2 depicts the ¹H NMR spectrum (500 MHZ, DMSO-d₆) of Compound 8.

FIG. 3 depicts the stability of Compound 8 in PBS (pH 7.4) containing 5.7 mM dithionite following exposure to air. Spectra were acquired at 600 s intervals once dithionite consumption was complete.

FIG. 4 depicts IR spectra (KBr pellet) of Compound 6 and the precipitate formed from Compound 8 and (PPh₄) Cl.

FIG. 5 depicts IR spectra (KBr pellet) of Compound 6 and the precipitate formed from Compound 8 and (PPh₄) Cl.

FIG. 6 depicts a ball-and-stick representation of Compound 5 from diffraction data confirming connectivity.

FIG. 7 depicts a ball-and-stick representation of Compound 6 from diffraction data confirming connectivity.

FIG. 8 depicts the titration of bovine COHb (2.5 μM) with 7 (produced in situ) in PBS (pH 7.4, 5.7 mM Na₂S₂O₄).

FIG. 9 depicts the titration of an equimolar (on the basis of porphyrin centers) mixture of Hb and Compound 7 in PBS (pH 7.4, 5.7 mM Na₂S₂O₄) with CO-saturated water. At 0 equiv CO, the mixture contains deoxyHb and Compound 7. At 1 equiv CO, the mixture contains deoxyHb and Compound 8. At 2 equiv CO, the mixture contains COHb and Compound 8.

FIG. 10 depicts hemolysis as assessed by measuring OD₇₀₀ over time of a suspension of RBCs in PBS (pH 7.4, 5.7 mM Na₂S₂O₄) containing no further additives, an equimolar (on the basis of porphyrin centers) amount of Compound 7, or 1.5 M NH₄Cl.

FIG. 11 depicts titration of a PBS suspension (pH 7.4, 5.7 mM Na₂S₂O₄) of CO-treated bovine RBCs with Compound 7 (produced in situ). Final trace obtained after bubbling CO through the suspension treated with 1.00 equiv of Compound 7.

FIG. 12 depicts the decrease in COHb (λₘₐₓ=420 nm) over time following addition of Compound 7 to a PBS suspension (pH 7.4, 5.7 mM Na₂S₂O₄) of CO-treated RBCs at an equimolar amount on the basis of porphyrin centers.

FIG. 13 depicts a schematic overview of a water soluble metal porphyrin complex for binding CO (e.g., as an antidote to CO poisoning) according to certain embodiments.

FIG. 14 depicts a mechanism of transfer of intracellular CO to extracellular water soluble metal porphyrin complexes according to certain embodiments.

FIG. 15 depicts Scheme 1 showing the synthesis of water soluble metal porphyrin complexes 6 and 7 according to certain embodiments.

FIG. 16 depicts a ball-and-stick representation of Compound 5 from diffraction data confirming connectivity.

FIG. 17 depicts a HPLC chromatogram of Compound 6. Absorbance is measured at 433 nm and the analyte was eluted with a H₂O/MeCN (0.01% TFA) gradient of 0-95% MeCN over 15 min.

FIG. 18 depicts the molecular structure (50% ellipsoids) of the anion obtained upon slow recrystallization of Compound 6 from DMSO/CHCl₃. H atoms, solvent, and counterions omitted for clarity.

FIG. 19 depicts the electronic absorption spectra of 10 μM solutions of Compounds 6, 7, and 8 in PBS (pH 7.4). For Compounds 7 and 8, the solutions also contain 5.7 mM Na₂S₂O₄.

FIG. 20 depicts reaction of Compound 7 with CO to produce Compound 8.

FIG. 21 depicts a general scheme for bis-pocket porphyrin synthesis according to certain embodiments.

FIGS. 22A-22X depict the NMR spectra for Compound 2-2b (FIG. 22A), Compound 2-2c (FIG. 22B), Compound 2-2d (FIG. 22C), Compound 2-2e (FIG. 22D), Compound 2-2f (FIG. 22E), Compound 2-2g (FIG. 22F), Compound 2-2h (FIG. 22G), Compound 2-2i (FIG. 22H), Compound 2-2j (FIG. 22I), Compound 2-2k (FIG. 22J), Compound 2-21 (FIG. 22K), Compound 2-2m (FIG. 22L), Compound 2-2n (FIG. 22M), Compound 2-2o (FIG. 22N), Compound 2-2p (FIG. 22O), Compound 2-2q (FIG. 22P), Compound 2-3a (FIG. 22Q), Compound 2-3b (FIG. 22R), Compound 2-4a (FIG. 22S), Compound 2-4b (FIG. 22T), Compound 2-4c (FIG. 22U), Compound 2-4d (FIG. 22V), Compound 2-4e (FIG. 22W), Compound 2-4f (FIG. 22X).

FIGS. 23A-23T depict the thermal ellipsoid plot of the crystal structures for Compound 2-2b (FIG. 23A), Compound 2-2c (FIG. 23B), Compound 2-2d (FIG. 23C), Compound 2-2e (FIG. 23D), Compound 2-2f (FIG. 23E), Compound 2-2h (FIG. 23F), Compound 2-2i (FIG. 23G), Compound 2-2k (FIG. 23H), Compound 2-21 (FIG. 23I), Compound 2-2m (FIG. 23J), Compound 2-2o (FIG. 23K), Compound 2-2p (FIG. 23L), Compound 2-2q (FIG. 23M), Compound 2-3b (FIG. 23N), Compound 2-4a (FIG. 23O), Compound 2-4b (FIG. 23P), Compound 2-4c (FIG. 23Q), Compound 2-4d (FIG. 23R), Compound 2-4e (FIG. 23S) and Compound 2-4f (FIG. 23T).

FIG. 24 depicts an exploration of the scope of groups that can be coupled to the porphyrin framework according to the depicted reaction. Yields are isolated yields.

FIG. 25 depicts pockets of Compounds 2-2c, 2-2d, 2-2f, 2-2k, 2-2q, and 2-2i as calculated with POVME2 using atomic coordinates from single-crystal X-ray diffraction data. The molecules are shown as sticks with a green surface at the van der Waals distance. The pockets are depicted as purple mesh. Atomic color code: C grey, H white, O red, N blue, Si tan, Cl green, F light green. Molecular graphics and analyses performed with UCSF ChimeraX.

FIG. 26 depicts sulfonation of bulky bis-pocket porphyrins. At right, thermal ellipsoid plot (50% probability level) of 2-3b with non-polar H atoms and three of the four Na⁺-diglyme complexes omitted for clarity. Color code: O red, N blue, Cl green, Na teal, C grey, and H white spheres of arbitrary radius.

FIG. 27 depicts metal insertion into bulky bis-pocket porphyrins.

FIG. 28. Thermal ellipsoid plots (50% probability level) of (A) the Zn-aqua complex 2-4a, (B) the Cu complex 2-4b (note that Pd complex 2-4c and the Co complex 2-4d are isomorphous), (C) the Fe-chloro complex 2-4e, and (D) the Fe-chloro complex 2-4f. H atoms, disorder, and solvent molecules are omitted for clarity. Color code: C grey, Si orange, O red, N blue, Cl green, Metal purple.

### DEFINITIONS

The following terms have the following meaning unless otherwise indicated. Any undefined terms have their art recognized meanings.

As used herein, the term “alkyl” by itself or as part of another substituent refers to a saturated branched or straight-chain monovalent hydrocarbon radical derived by the removal of one hydrogen atom from a single carbon atom of a parent alkane. Typical alkyl groups include, but are not limited to, methyl; ethyl, propyls such as propan-1-yl or propan-2-yl; and butyls such as butan-1-yl, butan-2-yl, 2-methyl-propan-1-yl or 2-methyl-propan-2-yl. In some embodiments, an alkyl group comprises from 1 to 20 carbon atoms. In other embodiments, an alkyl group comprises from 1 to 10 carbon atoms. In still other embodiments, an alkyl group comprises from 1 to 6 carbon atoms, such as from 1 to 4 carbon atoms.

“Alkanyl” by itself or as part of another substituent refers to a saturated branched, straight-chain or cyclic alkyl radical derived by the removal of one hydrogen atom from a single carbon atom of an alkane. Typical alkanyl groups include, but are not limited to, methanyl; ethanyl; propanyls such as propan-1-yl, propan-2-yl (isopropyl), cyclopropan-1-yl, etc.; butanyls such as butan-1-yl, butan-2-yl (sec-butyl), 2-methyl-propan-1-yl (isobutyl), 2-methyl-propan-2-yl (t-butyl), cyclobutan-1-yl, etc.; and the like.

“Alkylene” refers to a branched or unbranched saturated hydrocarbon chain, usually having from 1 to 40 carbon atoms, more usually 1 to 10 carbon atoms and even more usually 1 to 6 carbon atoms. This term is exemplified by groups such as methylene (—CH₂—), ethylene (—CH₂CH₂—), the propylene isomers (e.g., —CH₂CH₂CH₂— and —CH(CH₃)CH₂—) and the like. “Alkenyl” by itself or as part of another substituent refers to an unsaturated branched, straight-chain or cyclic alkyl radical having at least one carbon-carbon double bond derived by the removal of one hydrogen atom from a single carbon atom of an alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

“Alkynyl” by itself or as part of another substituent refers to an unsaturated branched, straight-chain or cyclic alkyl radical having at least one carbon-carbon triple bond derived by the removal of one hydrogen atom from a single carbon atom of an alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like. “Acyl” by itself or as part of another substituent refers to a radical-C (O) R³⁰, where R³⁰ is hydrogen, alkyl, cycloalkyl, cycloheteroalkyl, aryl, arylalkyl, heteroalkyl, heteroaryl, heteroarylalkyl as defined herein and substituted versions thereof. Representative examples include, but are not limited to formyl, acetyl, cyclohexylcarbonyl, cyclohexylmethylcarbonyl, benzoyl, benzylcarbonyl, piperonyl, succinyl, and malonyl, and the like.

The term “aminoacyl” refers to the group-C (O) NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Alkoxy” by itself or as part of another substituent refers to a radical —OR31 where R31 represents an alkyl or cycloalkyl group as defined herein. Representative examples include, but are not limited to, methoxy, ethoxy, propoxy, butoxy, cyclohexyloxy and the like.

“Alkoxycarbonyl” by itself or as part of another substituent refers to a radical —C(O)OR31 where R31 represents an alkyl or cycloalkyl group as defined herein. Representative examples include, but are not limited to, methoxycarbonyl, ethoxycarbonyl, propoxycarbonyl, butoxycarbonyl, cyclohexyloxycarbonyl and the like.

“Aryl” by itself or as part of another substituent refers to a monovalent aromatic hydrocarbon radical derived by the removal of one hydrogen atom from a single carbon atom of an aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. In certain embodiments, an aryl group comprises from 6 to 20 carbon atoms. In certain embodiments, an aryl group comprises from 6 to 12 carbon atoms. Examples of an aryl group are phenyl and naphthyl.

“Arylalkyl” by itself or as part of another substituent refers to an acyclic alkyl radical in which one of the hydrogen atoms bonded to a carbon atom, typically a terminal or sp3 carbon atom, is replaced with an aryl group. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2- naphthophenylethan-1-yl and the like. Where specific alkyl moieties are intended, the nomenclature arylalkanyl, arylalkenyl and/or arylalkynyl is used. In certain embodiments, an arylalkyl group is (C₇-C₃₀) arylalkyl, e.g., the alkanyl, alkenyl or alkynyl moiety of the arylalkyl group is (C₁-C₁₀) and the aryl moiety is (C₆-C₂₀). In certain embodiments, an arylalkyl group is (C₇-C₂₀) arylalkyl, e.g., the alkanyl, alkenyl or alkynyl moiety of the arylalkyl group is (C₁-C₈) and the aryl moiety is (C₆-C₁₂).

“Arylaryl” by itself or as part of another substituent, refers to a monovalent hydrocarbon group derived by the removal of one hydrogen atom from a single carbon atom of a ring system in which two or more identical or non-identical aromatic ring systems are joined directly together by a single bond, where the number of such direct ring junctions is one less than the number of aromatic ring systems involved. Typical arylaryl groups include, but are not limited to, biphenyl, triphenyl, phenyl-napthyl, binaphthyl, biphenyl-napthyl, and the like. When the numbers of carbon atoms in an arylaryl group are specified, the numbers refer to the carbon atoms comprising each aromatic ring. For example, (C₅-C₁₄) arylaryl is an arylaryl group in which each aromatic ring comprises from 5 to 14 carbons, e.g., biphenyl, triphenyl, binaphthyl, phenylnapthyl, etc. In certain embodiments, each aromatic ring system of an arylaryl group is independently a (C₅-C₁₄) aromatic. In certain embodiments, each aromatic ring system of an arylaryl group is independently a (C₅-C₁₀) aromatic. In certain embodiments, each aromatic ring system is identical, e.g., biphenyl, triphenyl, binaphthyl, trinaphthyl, etc. “Cycloalkyl” by itself or as part of another substituent refers to a saturated or unsaturated cyclic alkyl radical. Where a specific level of saturation is intended, the nomenclature “cycloalkanyl” or “cycloalkenyl” is used. Typical cycloalkyl groups include, but are not limited to, groups derived from cyclopropane, cyclobutane, cyclopentane, cyclohexane and the like. In certain embodiments, the cycloalkyl group is (C₃-C₁₀) cycloalkyl. In certain embodiments, the cycloalkyl group is (C₃-C₇) cycloalkyl. “Cycloheteroalkyl” or “heterocyclyl” by itself or as part of another substituent, refers to a saturated or unsaturated cyclic alkyl radical in which one or more carbon atoms (and any associated hydrogen atoms) are independently replaced with the same or different heteroatom. Typical heteroatoms to replace the carbon atom(s) include, but are not limited to, N, P, O, S, Si, etc. Where a specific level of saturation is intended, the nomenclature “cycloheteroalkanyl” or “cycloheteroalkenyl” is used. Typical cycloheteroalkyl groups include, but are not limited to, groups derived from epoxides, azirines, thiiranes, imidazolidine, morpholine, piperazine, piperidine, pyrazolidine, pyrrolidine, quinuclidine and the like.

“Heteroalkyl, Heteroalkanyl, Heteroalkenyl and Heteroalkynyl” by themselves or as part of another substituent refer to alkyl, alkanyl, alkenyl and alkynyl groups, respectively, in which one or more of the carbon atoms (and any associated hydrogen atoms) are independently replaced with the same or different heteroatomic groups. Typical heteroatomic groups which can be included in these groups include, but are not limited to, —O—, —S—, —S—S—, —O—S—, —NR³⁷R³⁸—, ═N—N═, —N═N—, —N═N—NR³⁹R⁴⁰, —PR⁴¹—, —P(O)₂—, —POR⁴²—, —O—P(O)₂—, —S—O—, —S—(O)—, —SO₂—, —SnR⁴³R⁴⁴— and the like, where R³⁷, R³⁸, R³⁹, R⁴⁰, R⁴¹, R⁴², R⁴³ and R⁴⁺are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl or substituted heteroarylalkyl.

“Heteroaryl” by itself or as part of another substituent, refers to a monovalent heteroaromatic radical derived by the removal of one hydrogen atom from a single atom of a heteroaromatic ring system. Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, benzodioxole and the like. In certain embodiments, the heteroaryl group is from 5-20 membered heteroaryl. In certain embodiments, the heteroaryl group is from 5-10 membered heteroaryl. In certain embodiments, heteroaryl groups are those derived from thiophene, pyrrole, benzothiophene, benzofuran, indole, pyridine, quinoline, imidazole, oxazole and pyrazine. “Heteroarylalkyl” by itself or as part of another substituent, refers to an acyclic alkyl radical in which one of the hydrogen atoms bonded to a carbon atom, typically a terminal or sp3 carbon atom, is replaced with a heteroaryl group. Where specific alkyl moieties are intended, the nomenclature heteroarylalkanyl, heteroarylalkenyl and/or heterorylalkynyl is used. In certain embodiments, the heteroarylalkyl group is a 6-30 membered heteroarylalkyl, e.g., the alkanyl, alkenyl or alkynyl moiety of the heteroarylalkyl is 1-10 membered and the heteroaryl moiety is a 5-20-membered heteroaryl. In certain embodiments, the heteroarylalkyl group is 6-20 membered heteroarylalkyl, e.g., the alkanyl, alkenyl or alkynyl moiety of the heteroarylalkyl is 1-8 membered and the heteroaryl moiety is a 5-12-membered heteroaryl.

“Aromatic Ring System” by itself or as part of another substituent, refers to an unsaturated cyclic or polycyclic ring system having a conjugated x electron system. Specifically included within the definition of “aromatic ring system” are fused ring systems in which one or more of the rings are aromatic and one or more of the rings are saturated or unsaturated, such as, for example, fluorene, indane, indene, phenalene, etc. Typical aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. “Heteroaromatic Ring System” by itself or as part of another substituent, refers to an aromatic ring system in which one or more carbon atoms (and any associated hydrogen atoms) are independently replaced with the same or different heteroatom. Typical heteroatoms to replace the carbon atoms include, but are not limited to, N, P, O, S, Si, etc. Specifically included within the definition of “heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, etc. Typical heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene and the like.

“Substituted” refers to a group in which one or more hydrogen atoms are independently replaced with the same or different substituent(s). Typical substituents include, but are not limited to, alkylenedioxy (such as methylenedioxy), —M, —R⁶⁰, —O⁻, ═O, —OR⁶⁰, —SR⁶⁰, —S⁻, ═S, —NR⁶⁰R⁶¹, ═NR⁶⁰, —CF₃, —CN, —OCN, —SCN, —NO, —NO₂, ═N₂, —N₃, —S(O)₂O⁻, —S(O)₂OH, —S(O)₂R⁶⁰, —OS(O)₂O⁻; —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O⁻, —C(S)OR⁶⁰, —NR⁶²C(O)NR⁶⁰R⁶¹, —NR⁶²C(S)NR⁶⁰R⁶¹, —NR⁶²C(NR⁶³)NR⁶⁰R⁶¹ and —C(NR⁶²)NR⁶⁰R⁶¹ where M is halogen; R⁶⁰, R⁶¹, R⁶² and R⁶³ are independently hydrogen, alkyl, substituted alkyl, alkoxy, substituted alkoxy, cycloalkyl, substituted cycloalkyl, cycloheteroalkyl, substituted cycloheteroalkyl, aryl, substituted aryl, heteroaryl or substituted heteroaryl, or optionally R⁶⁰ and R⁶¹ together with the nitrogen atom to which they are bonded form a cycloheteroalkyl or substituted cycloheteroalkyl ring; and R⁶⁴ and R⁶⁵ are independently hydrogen, alkyl, substituted alkyl, aryl, cycloalkyl, substituted cycloalkyl, cycloheteroalkyl, substituted cycloheteroalkyl, aryl, substituted aryl, heteroaryl or substituted heteroaryl, or optionally R⁶⁴ and R⁶⁵ together with the nitrogen atom to which they are bonded form a cycloheteroalkyl or substituted cycloheteroalkyl ring. In certain embodiments, substituents include —M, —R⁶⁰, ═O, —OR⁶⁰, —SR⁶⁰, —S⁻, ═S, —NR⁶⁰R⁶¹, ═NR⁶⁰, —CF₃, —CN, —OCN, —SCN, —NO, —NO₂, ═N₂, —N₃, —S(O)₂R⁶⁰, —OS(O)₂O⁻, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O⁻, —NR⁶²C(O)NR⁶⁰R⁶¹. In certain embodiments, substituents include —M, —R⁶⁰, ═O, —OR⁶⁰, —SR⁶⁰, —NR⁶⁰R⁶¹, —CF₃, —CN, —NO₂, —S(O)₂R⁶⁰, —P(O)(OR⁶⁰)(O⁻), —OP(O)(OR⁶⁰)(OR⁶¹), —C(O)R⁶⁰, —C(O)OR⁶⁰, —C(O)NR⁶⁰R⁶¹, —C(O)O⁻. In certain embodiments, substituents include —M, —R⁶⁰, ═O, —OR⁶⁰, —SR⁶⁰, —NR⁶⁰R⁶¹, —CF₃, —CN, —NO₂, —S(O)₂R⁶⁰, —OP(O)(OR⁶⁰)(OR⁶¹), —C(O)R⁶⁰, —C(O)OR⁶⁰, —C(O)O⁻, where R⁶⁰, R⁶¹ and R⁶² are as defined above. For example, a substituted group may bear a methylenedioxy substituent or one, two, or three substituents selected from a halogen atom, a (1-4C) alkyl group and a (1-4C) alkoxy group.

“Pharmaceutically acceptable carrier” refers to a diluent, adjuvant, excipient or vehicle with, or in which a compound is administered.

### DETAILED DESCRIPTION

Compounds for binding carbon monoxide are provided (e.g., to sequest carbon monoxide in a composition). Compounds according to certain embodiments include water soluble metal porphyrin complexes having substituents that provide for water solubility at physiological pH and form a hydrophobic carbon monoxide (CO)-binding pocket. In certain embodiments, metal porphyrin complexes exhibit limited cellular uptake. In some instances, the compounds described herein are capable of rescuing CO-poisoned red blood cells. Methods for treating a subject exposed to carbon monoxide (e.g., experiencing carbon monoxide poisoning) are also provided. Compositions for practicing the subject methods are also described.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the compounds and methods have or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the chemical groups represented by the variables are specifically embraced by the present invention and are disclosed herein just as if each and every combination was individually and explicitly disclosed, to the extent that such combinations embrace compounds that are stable compounds (i.e., compounds that can be isolated, characterised, and tested for biological activity). In addition, all sub-combinations of the chemical groups listed in the embodiments describing such variables are also specifically embraced by the present invention and are disclosed herein just as if each and every such sub-combination of chemical groups was individually and explicitly disclosed herein.

Reference will now be made in detail to various embodiments. It will be understood that the invention is not limited to these embodiments. To the contrary, it is intended to cover alternatives, modifications, and equivalents as may be included within the spirit and scope of the allowed claims.

### Compounds for Binding Carbon Monoxide

In some embodiments, compounds of the present disclosure include a compound of formula (I):

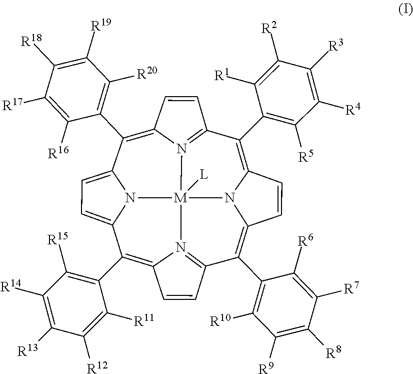

wherein:

R², R⁴, R⁷, R⁹, R¹², R¹⁴, R¹⁷ and R¹⁹ are each independently selected from hydrogen, hydroxy, alkoxy, amine, cyano, thiol, halogen, alkyl, substituted alkyl, haloalkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;

R¹, R⁵, R⁶, R¹⁰, R¹¹, R¹⁵, R¹⁶ and R²⁰ are each independently selected from substituted alkyl, haloalkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;

R³, R⁸, R¹³ and R¹⁸ are each independently a water soluble group;

M is a metal; and

L is a ligand,

or a salt, solvate or hydrate thereof.

In embodiments, “salts” of the compounds of the present disclosure may include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo [2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the compound is replaced by a metal ion, e.g., an alkali metal ion, an alkaline earth ion, or an aluminum ion; or coordinates with an organic base such as ethanolamine, diethanolamine, triethanolamine, N-methylglucamine and the like. In some embodiments, salts of interest include sodium salts.

The term “solvate” as used herein refers to a complex or aggregate formed by one or more molecules of a solute, e.g. a compound of Formula (I) or a salt thereof, and one or more molecules of a solvent. Such solvates may be crystalline solids having a substantially fixed molar ratio of solute and solvent. Representative solvents include by way of example, water, methanol, ethanol, isopropanol, acetic acid, and the like. When the solvent is water, the solvate formed is a hydrate.

In some embodiments, R² is hydrogen. In some embodiments, R⁴ is hydrogen. In some embodiments, R⁷ is hydrogen. In some embodiments, R⁹ is hydrogen. In some embodiments, R¹² is hydrogen. In some embodiments, R¹⁴ is hydrogen. In some embodiments, R¹⁷ is hydrogen. In some embodiments, R¹⁹ is hydrogen.

In some embodiments, R² is a C(1-6)alkyl. In some instances, R² is selected from methyl, ethyl, n-propyl, isopropyl, n-butyl, t-butyl, pentyl and hexyl. In some embodiments, R⁴is a C (1-6) alkyl. In some instances, R⁴ is selected from methyl, ethyl, n-propyl, isopropyl, n-butyl, t-butyl, pentyl and hexyl. In some embodiments, R⁷ is a C(1-6)alkyl. In some instances, R⁷ is selected from methyl, ethyl, n-propyl, isopropyl, n-butyl, t-butyl, pentyl and hexyl. In some embodiments, R⁹ is a C(1-6)alkyl. In some instances, R⁹ is selected from methyl, ethyl, n-propyl, isopropyl, n-butyl, t-butyl, pentyl and hexyl. In some embodiments, R¹² is a C(1-6)alkyl. In some instances, R¹² is selected from methyl, ethyl, n-propyl, isopropyl, n-butyl, t-butyl, pentyl and hexyl. In some embodiments, R¹⁴ is a C(1-6)alkyl. In some instances, R¹⁴ is selected from methyl, ethyl, n-propyl, isopropyl, n-butyl, t-butyl, pentyl and hexyl. In some embodiments, R¹⁷ is a C(1-6)alkyl. In some instances, R¹⁷ is selected from methyl, ethyl, n-propyl, isopropyl, n-butyl, t-butyl, pentyl and hexyl. In some embodiments, R¹⁹ is a C(1-6)alkyl. In some instances, R¹⁹ is selected from methyl, ethyl, n-propyl, isopropyl, n-butyl, t-butyl, pentyl and hexyl.

In embodiments, R¹, R⁵, R⁶, R¹⁰, R¹¹, R¹⁵, R¹⁶ and R²⁰ are each independently a bulky group. In some embodiments, the bulky group is a hydrophobic group, such as for where R¹, R⁵, R⁶, R¹⁰, R¹¹, R¹⁵, R¹⁶ and R²⁰ provide for a hydrophobic binding pocket for carbon monoxide to the metal. In some embodiments, R¹ is an aryl group. In some instances, R¹ is phenyl. In some embodiments, R⁵ is an aryl group. In some instances, R⁵ is phenyl. In some embodiments, R⁶ is an aryl group. In some instances, R⁶ is phenyl. In some embodiments, R¹⁰ is an aryl group. In some instances, R¹⁰ is phenyl. In some embodiments, R¹¹ is an aryl group. In some instances, R¹¹ is phenyl. In some embodiments, R¹⁵ is an aryl group. In some instances, R¹⁵ is phenyl. In some embodiments, R¹⁶ is an aryl group. In some instances, R¹⁶ is phenyl. In some embodiments, R²⁰ is an aryl group. In some instances, R²⁰ is phenyl.

In some embodiments, R¹, R⁵, R⁶, R¹⁰, R¹¹, R¹⁵, R¹⁶ and R²⁰ are each independently selected from the group consisting of:

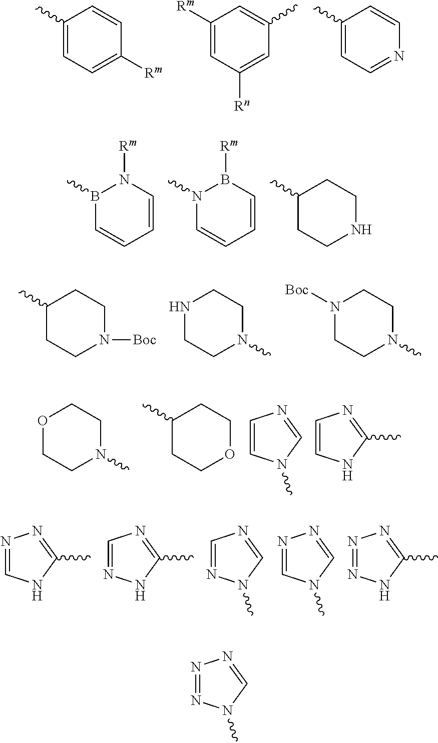

where R^{m} and Rⁿ are independently selected from hydrogen, halogen, hydroxyl, substituted hydroxyl, amino, substituted amino, thiol, substituted thiol, sulfoxide, substituted sulfoxide, sulfone, substituted sulfone, sulfoximine, substituted sulfoximine. acyl, aminoacyl, alkyl, substituted alkyl; heteroalkyl, substituted heteroalkyl, cycloalkyl, substituted cycloalkyl, spiroalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl.

In certain embodiments, R¹, R⁵, R⁶, R¹⁰, R¹¹, R¹⁵, R¹⁶ and R²⁰ are each independently selected from the group consisting of:

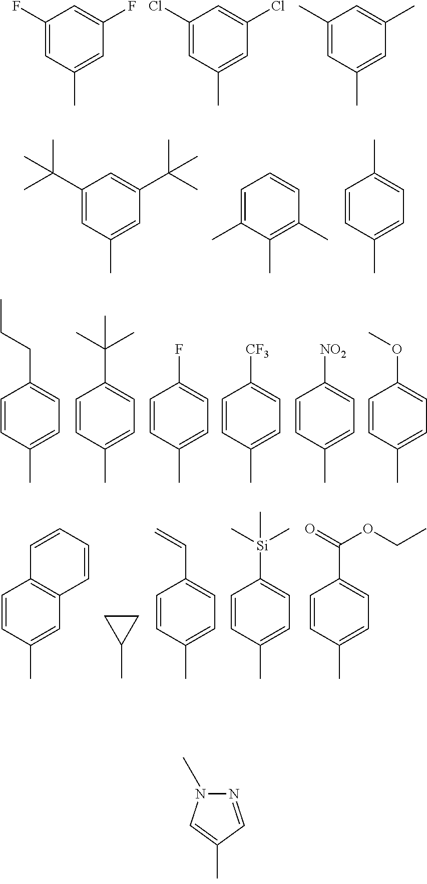

In embodiments, R³, R⁸, R¹³ and R¹⁸ are each a group that increase the water solubility of the compound. In some instances, one or more of R³, R⁸, R¹³ and R¹⁸ are each a cationic group. In some instances, one or more of R³, R⁸, R¹³ and R¹⁸ are each an anionic group. In some instances, one or more of R³, R⁸, R¹³ and R¹⁸ are each a zwitterionic group. In certain instances, one or more of R³, R⁸, R¹³ and R¹⁸ are each independently selected from sulfonate, carboxylate, ammonium, trialkylammonium, pyridinium, N-alkylpyridinium, or poly (ethylene glycol). In some instances, one or more of R³, R⁸, R¹³ and R¹⁸ are sulfonate. In some instances, one or more of R³, R⁸, R¹³ and R¹⁸ are carboxylate. In some instances, one or more of R³, R⁸, R¹³ and R¹⁸ are 10 ammonium. In some instances, one or more of R³, R⁸, R¹³ and R¹⁸ are trialkylammonium. In some instances, one or more of R³, R⁸, R¹³ and R¹⁸ are pyridinium. In some instances, one or more of R³, R⁸, R¹³ and R¹⁸ are N-alkylpyridinium. In some instances, one or more of R³, R⁸, R¹³ and R¹⁸ are a polyalkylene glycol. In certain instances, one or more of R³, R⁸, R¹³ and R 18 are poly (ethylene glycol).

In some embodiments, M is a metal selected from the group consisting of Ti, Cr, Fe, Co, Ni, Cu, Zn, Mn, Sn, Pb, Mo, Ru, Rh, Pd, Cd, Pt, Ir, and Os. In some embodiments, M in the metal porphyrin complexes is Fe. In some instances, M is Ti. In some instances, M is Cr. In some instances, M is Co. In some instances, M is Ni. In some instances, M is Cu. In some instances, M is Zn. In some instances, M is Mn. In some instances, M is Sn. In some instances, M is Pb. In some instances, M is Mo. In some instances, M is Ru. In some instances, M is Rh. In some instances, M is Pd. In some instances, M is Cd. In some instances, M is Pt. In some instances, M is Ir. In some instances, M is Os.

In some embodiments, L is hydroxy. In some embodiments, L is alkoxy. In some embodiments, L is a C(1-6)alkoxy. In some embodiments, L is an alkylthiolate. In some embodiments, L is an arylthiolate. In some embodiments, L is a substituted arylthiolate. In some embodiments, L is a thiolate-bearing peptide. In certain instances, L is cysteine.

In some embodiments, the compound is a salt. In certain instances, the compound is a tetrasodium salt. In certain instances, the compound is sodium 5,10,15,20-tetrakis(2,6-diphenyl-4-(sulfonate)phenyl)porphyrinatohydroxoiron(III):

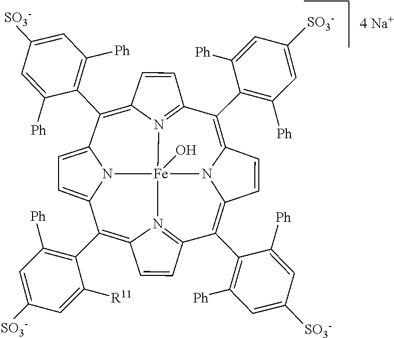

In certain embodiments, the compound is formed from contacting sodium 5,10,15,20-tetrakis (2,6-diphenyl-4-(sulfonate)phenyl)porphyrinatohydroxoiron(III) with a reducing agent. In some instances, the reducing agent is a reductant selected from the group consisting of alkylthiols, substituted alkylthiols, arylthiols, substituted arylthiols, thiol-bearing amino acids (e.g., cysteine) and thiol-bearing peptides (e.g. glutathione).

In certain instances, the reducing agent contacted with the compound is sodium dithionite to form a compound of formula (II):

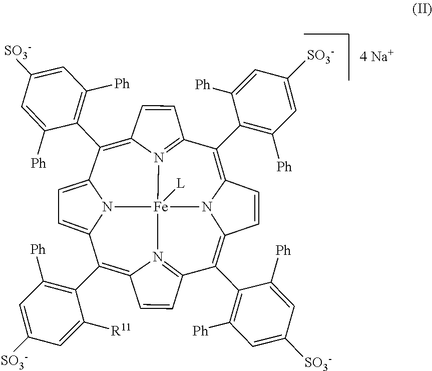

In certain instances, the compound is selected from:

5,10,15,20-tetrakis(2,6-diphenyl-4-(trimethylsilyl)phenyl)porphyrin;

5,10,15,20-tetrakis(2,6-di(3,5-difluoro)phenyl-4-(trimethylsilyl) phenyl) porphyrin;

5,10,15,20-tetrakis(2,6-di(3,5-dichloro)phenyl-4-(trimethylsilyl) phenyl) porphyrin;

5,10,15,20-tetrakis(2,6-di(3,5-dimethyl)phenyl-4-(trimethylsilyl)phenyl)porphyrin;

5,10,15,20-tetrakis(2,6-di(4-methyl)phenyl-4-(trimethylsilyl)phenyl)porphyrin;

5,10,15,20-tetrakis(2,6-di(4-n-propyl)phenyl-4-(trimethylsilyl)phenyl)porphyrin;

5,10,15,20-tetrakis(2,6-di(4-tert-butyl)phenyl-4-(trimethylsilyl)phenyl)porphyrin;

5,10,15,20-tetrakis(2,6-di(4-fluoro)phenyl-4-(trimethylsilyl)phenyl)porphyrin;

5,10,15,20-tetrakis(2,6-di(4-trifluoromethyl)phenyl-4-(trimethylsilyl)phenyl)porphyrin;

5,10,15,20-tetrakis(2,6-di(4-nitro)phenyl-4-(trimethylsilyl)phenyl)porphyrin;

5,10,15,20-tetrakis(2,6-di(4-methoxy)phenyl-4-(trimethylsilyl)phenyl)porphyrin;

5,10,15,20-tetrakis(2,6-di(2-napthyl)phenyl-4-(trimethylsilyl)phenyl)porphyrin;

5,10,15,20-tetrakis(2,6-dicyclopropyl-4-(trimethylsilyl)phenyl)porphyrin;

5,10,15,20-tetrakis(2,6-d (4-vinyl)phenyl-4-(trimethylsilyl)phenyl)porphyrin;

5,10,15,20-tetrakis(2,6-di(4-trimethylsilyl)phenyl-4-(trimethylsilyl)phenyl)porphyrin;

5,10, 15,20-tetrakis(2,6-di(4-ethoxycarbonyl)phenyl-4-(trimethylsilyl)phenyl)porphyrin;

5,10,15,20-tetrakis(2,6-di(N-methylpyrrazolyl)phenyl-4-(trimethylsilyl)phenyl)porphyrin;

5,10,15,20-tetrakis(2,6-diphenyl-4-(sulfonato)phenyl)porphyrin;

5,10,15,20-tetrakis(2,6-di(3,5-difluoro) phenyl-4-(sulfonato)phenyl)porphyrin;

5,10,15,20-tetrakis(2,6-diphenyl-4-(trimethylsilyl)phenyl)porphyrinatoaquazinc(II);

5,10,15,20-tetrakis(2,6-diphenyl-4-(trimethylsilyl)phenyl)porphyrinatocopper(II);

5,10,15,20-tetrakis(2,6-diphenyl-4-(trimethylsilyl)phenyl)porphyrinatopalladium(II);

5,10,15,20-tetrakis(2,6-diphenyl-4-(trimethylsilyl)phenyl)porphyrinatocobalt(II);

5,10,15,20-tetrakis(2,6-diphenyl-4-(trimethylsilyl)phenyl)porphyrinatochloroiron(III); and

5,10,15,20-tetrakis(2,6-di(3,5-dimethyl)phenyl-4-(trimethylsilyl)phenyl)porphyrinatochloroiron(III)

Aspects of the present disclosure also include compositions having a pharmaceutically acceptable carrier and one or more of the compounds described above. A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7th ed., Lippincott, Williams, & Wilkins; and Handbook of Pharmaceutical Excipients (2000) A. H. Kibbe et al., eds., 3rd ed. Amer.

Pharmaceutical Assoc. For example, the one or more excipients may include sucrose, starch, mannitol, sorbitol, lactose, glucose, cellulose, talc, calcium phosphate or calcium carbonate, a binder (e.g., cellulose, methylcellulose, hydroxymethylcellulose, polypropylpyrrolidone, polyvinylpyrrolidone, gelatin, gum arabic, poly (ethylene glycol), sucrose or starch), a disintegrator (e.g., starch, carboxymethylcellulose, hydroxypropyl starch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g., sodium benzoate, sodium bisulfite, methylparaben or propylparaben), a stabilizer (e.g., citric acid, sodium citrate or acetic acid), a suspending agent (e.g., methylcellulose, polyvinylpyrrolidone or aluminum stearate), a dispersing agent (e.g., hydroxypropylmethylcellulose), a diluent (e.g., water), and base wax (e.g., cocoa butter, white petrolatum or polyethylene glycol).

The compounds may be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols. In certain embodiments, the conjugate compounds are formulated for injection. For example, compositions of interest may be formulated for intravenous or intraperitoneal administration.

In pharmaceutical dosage forms, the compounds may be administered in the form of its pharmaceutically acceptable salts, or it may also be used alone or in appropriate association, as well as in combination, with other pharmaceutically active compounds. The following methods and excipients are merely exemplary and are in no way limiting.

In some embodiments, compositions of interest include an aqueous buffer. Suitable aqueous buffers include, but are not limited to, acetate, succinate, citrate, and phosphate buffers varying in strengths from about 5 mM to about 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. In some instances, compositions of interst further include a preservative. Suitable preservatives include, but are not limited to, a benzyl alcohol, phenol, chlorobutanol, benzalkonium chloride, and the like. In many cases, the composition is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures.

In some embodiments, compositions include other additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Where the composition is formulated for injection, the compounds may be formulated by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Although the dosage used in treating a subject will vary depending on the clinical goals to be achieved, a suitable dosage range of the compound is one which provides up to about 0.0001 mg to about 5000 mg, e.g., from about 1 mg to about 25 mg, from about 25 mg to about 50 mg, from about 50 mg to about 100 mg, from about 100 mg to about 200 mg, from about 200 mg to about 250 mg, from about 250 mg to about 500 mg, from about 500 mg to about 1000 mg, or from about 1000 mg to about 5000 mg of an active agent, which can be administered in a single dose. Those of skill will readily appreciate that dose levels can vary as a function of the specific compound, the severity of the symptoms and the susceptibility of the subject to side effects.

In some embodiments, a single dose of the compound is administered. In other embodiments, multiple doses of the compound are administered. Where multiple doses are administered over a period of time, the compound may be administered, e.g., twice daily (qid), daily (qd), every other day (qod), every third day, three times per week (tiw), or twice per week (biw) over a period of time. For example, the compound may be administered qid, qd, qod, tiw, or biw over a period of from one day to about 2 years or more. For example, the compound may be administered at any of the aforementioned frequencies for one week, two weeks, one month, two months, six months, one year, or two years, or more, depending on various factors.

Dose units of the present disclosure can be made using manufacturing methods available in the art and can be of a variety of forms suitable for injection (including topical, intracisternal, intrathecal, intravenous, intramuscular, subcutaneous and dermal) administration, for example as a solution, suspension, solution, lyophilate or emulsion. The dose unit can contain components conventional in pharmaceutical preparations, e.g. one or more carriers, binders, lubricants, excipients (e.g., to impart controlled release characteristics), pH modifiers, coloring agents or further active agents.

Dose units can comprise components in any relative amounts. For example, dose units can be from about 0.1% to 99% by weight of active ingredients (i.e., compounds described herein) per total weight of dose unit. In some embodiments, dose units can be from 10% to 50%, from 20% to 40%, or about 30% by weight of active ingredients per total weight dose unit.

### Methods for Binding Carbon Monoxide

As summarized above, aspects of the present disclosure also include methods for binding carbon monoxide with one or more of the compounds described herein. In certain embodiments, methods include sequestering carbon monoxide with one or more of the water soluble metal porphyrin complexes. In some embodiments, the subject compounds are contacted with a composition in a manner sufficient to bind to free carbon monoxide in a composition. The term “free carbon monoxide” is used herein in its conventional sense to refer to carbon monoxide which is not chemically (e.g., through covalent bonds) or physically associated with (e.g., through hydrogen bonding or dipole-dipole interactions) with a compound in the composition. For example, free carbon monoxide sequestered in the composition using the subject water soluble metal porphyrin complexes include carbon monoxide solubilized in the composition. In some embodiments, contacting the composition with the subject water soluble metal porphyrin complexes is sufficient to bind to 50% or more of the free carbon monoxide in the composition, such as 60% or more, such as 70% or more, such as 80% or more, such as 90% or more, such as 95% or more, such as 97% or more, such as 99% or more, such as 99.9% or more and including 99.99% or more of the free carbon monoxide in the composition.

In other embodiments, the subject compounds are contacted with a composition in a manner sufficient to transfer carbon monoxide that is bound to a component in a composition (e.g., carbon monoxide bound to a heme group in hemoglobin in a blood sample) to the subject water soluble metal porphyrin complexes. The term “bound” is used herein in its conventional sense to refer to carbon monoxide in the composition which is chemically (e.g., through covalent bonds) or physically associated with (e.g., through hydrogen bonding or dipole-dipole interactions) with a compound in the composition. In some instances, carbon monoxide that bound to a component in the composition is transferred to the water soluble metal porphyrin complexes by contacting the water soluble metal porphyrin complexes with the composition for 1 second or longer, such as 2 seconds or longer, such as 5 seconds or longer, such as 10 seconds or longer, such as 15 seconds or longer, such as 30 seconds or longer, such as 60 seconds or longer, such as for 2 minutes or longer, such as from 5 minutes or longer, such as for 10 minutes or longer, such as for 30 minutes or longer, such as for 60 minutes or longer. In certain instances, the water soluble metal porphyrin complexes described herein facilitate transfer of carbon monoxide from a bound component in the composition after contacting the composition with the water soluble metal porphyrin complexes for 1 hour or longer, such as 2 hours or longer, such as 6 hours or longer, such as 12 hours or longer, such as 18 hours or longer, such as 24 hours or longer and including for 48 hours or longer. In some embodiments, contacting the composition with the subject water soluble metal porphyrin complexes is sufficient to bind to 50% or more of the carbon monoxide bound to a component in the composition (e.g., transfer carbon monoxide bound to a heme group in hemoglobin in a blood sample), such as 60% or more, such as 70% or more, such as 80% or more, such as 90% or more, such as 95% or more, such as 97% or more, such as 99% or more, such as 99.9% or more and including 99.99% or more of the carbon monoxide bound to a component in the composition.

In practicing methods according to certain embodiments, compositions contacted with the subject water soluble metal porphyrin complexes may be a biological sample. As used herein, the term “biological sample” is used in its conventional sense to refer to a whole organism, plant, fungi or a subset of animal tissues, cells or component parts which may in certain instances be found in blood, mucus, lymphatic fluid, synovial fluid, cerebrospinal fluid, saliva, bronchoalveolar lavage, amniotic fluid, amniotic cord blood, urine, vaginal fluid and semen. As such, a “biological sample” refers to both the native organism or a subset of its tissues as well as to a homogenate, lysate or extract prepared from the organism or a subset of its tissues, including but not limited to, for example, plasma, serum, spinal fluid, lymph fluid, sections of the skin, respiratory, gastrointestinal, cardiovascular, and genitourinary tracts, tears, saliva, milk, blood cells, tumors, organs.

In some embodiments, the sample is obtained from an in vivo source and can include samples obtained from tissues (e.g., cell suspension from a tissue biopsy, cell suspension from a tissue sample, etc.) and/or body fluids (e.g., whole blood, fractionated blood, plasma, serum, saliva, lymphatic fluid, interstitial fluid, etc.). In some cases, cells, fluids, or tissues derived from a subject are cultured, stored, or manipulated prior to evaluation. In vivo sources include living multi-cellular organisms and can yield non-diagnostic or diagnostic cellular samples. In certain embodiments the source of the sample is a “mammal” or “mammalian”, where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some instances, the subjects are humans. The methods may be applied to samples obtained from human subjects of both genders and at any stage of development (i.e., neonates, infant, juvenile, adolescent, adult), where in certain embodiments the human subject is a juvenile, adolescent or adult. While the present invention may be applied to samples from a human subject, it is to be understood that the methods may also be carried-out on samples from other animal subjects (that is, in “non-human subjects”) such as, but not limited to, birds, mice, rats, dogs, cats, livestock and horses.

In certain embodiments, the biological sample is a specimen that has been preloaded into a container (e.g., blender cup, vortex microtube, sonicator vessel, etc.) and is stored in the container for a predetermined period of time before contacting the biological sample with the water soluble metal porphyrin complexes. The amount of time the biological sample is stored following preloading into the container before contacting with the water soluble metal porphyrin complexes may vary, such as 0.1 hours or more, such as 0.5 hours or more, such as 1 hour or more, such as 2 hours or more, such as 4 hours or more, such as 8 hours or more, such as 16 hours or more, such as 24 hours or more. For example, the biological sample may be preloaded into a container (e.g., blender cup, vortex microtube, sonicator vessel, etc.) at a remote location (e.g., at home using an at-home kit or in a physician's office) and sent to a laboratory for processing in accordance with the subject methods. By “remote location” is meant a location other than the location at which the sample is contained and preloaded into the container. For example, a remote location could be another location (e.g. office, lab, etc.) in the same city, another location in a different city, another location in a different state, another location in a different country, etc., relative to the location of the processing device, e.g., as described in greater detail below. In some instances, two locations are remote from one another if they are separated from each other by a distance of 10 m or more, such as 50 m or more, including 100 m or more, e.g., 500 m or more, 1000 m or more, 10,000 m or more, up to, in some instances, 100,000 m, etc.

Biological samples processed by the subject methods may exhibit a wide range of viscosities. The viscosity of a liquid may depend on temperature. In certain embodiments, a fluid sample has a viscosity substantially equal to that of water at the given temperature (e.g., 1 cP at 20° C., 0.65 cP at 40° C.). Fluid samples useful in the present disclosure may exhibit a wide range of viscosities, ranging in some aspects from 0.01 cP to 750 cP, including 0.1 cP to 100 cP, such as 0.1 cP to 50 cP, 0.2 cP to 10 cP, 0.2 cP to 2.0 cP, 0.5 to 1.5 cP, or 0.75 cP to 1.5 cP.

Contacting the biological sample (e.g., whole blood sample) with the water soluble metal porphyrin complexes can be carried out at any suitable temperature so long as the viability of the cells collected is preserved as desired. As such, the temperature for contacting the biological sample with the water soluble metal porphyrin complexes may vary, such as from −80° C. to 100° C., such as from −75° C. to 75° C., such as from −50° C. to 50° C., such as from −25° C. to 25° C., such as from −10° C. to 10° C., and including from 0° C. to 25° C.

In some embodiments, methods include contacting the water soluble metal porphyrin complexes described herein in vitro. In other embodiments, methods include the water soluble metal porphyrin complexes in vivo (e.g., by administering to a subject as described in greater detail below). In still other embodiments, methods include the water soluble metal porphyrin complexes ex vivo (e.g., using an extracorporeal blood treatment system).

In some instances, methods include treating or preventing carbon monoxide exposure or poisoning. The term “treat” or “treatment” of any condition, refers, in certain embodiments, to ameliorating the condition (i.e., arresting or reducing the development of the condition). In certain embodiments “treating” or “treatment” refers to ameliorating at least one physical parameter, which may not be discernible by the patient. In certain embodiments, “treating” or “treatment” refers to inhibiting the condition, either physically, (e.g., stabilization of a discernible symptom), physiologically, (e.g., stabilization of a physical parameter), or both. In certain embodiments, “treating” or “treatment” refers to delaying the onset of the condition. The term “therapeutically effective amount” is used herein to refer to the amount of a compound that, when administered to a patient for preventing or treating a condition is sufficient to effect such treatment. The “therapeutically effective amount” will vary depending on the compound, the condition and its severity and the age, weight, etc., of the patient.

In practicing the subject methods, a therapeutically effective amount of one or more of the compounds disclosed herein is administered to a subject sufficient to treat carbon monoxide exposure or to prevent carbon monoxide poisoning. In embodiments, the term “subject” is meant the person or organism to which the compound is administered. As such, subjects of the present disclosure may include but are not limited to mammals, e.g., humans and other primates, such as chimpanzees and other apes and monkey species, dogs, rabbits, cats and other domesticated pets; and the like, where in certain embodiments the subject are humans. The term “subject” is also meant to include a person or organism of any age, weight or other physical characteristic, where the subjects may be an adult, a child, an infant or a newborn.

Compounds as described herein may be administered to a subject by any convenient protocol, including, but not limited, to intraperitoneally, topically, orally, sublingually, parenterally, intravenously, vaginally, rectally as well as by transdermal protocols. In certain embodiments, the subject compounds are administered by intravenous injection. In certain embodiments, the subject compounds are administered by intraperitoneal injection.

Depending on the condition being treated, the amount of compound administered to the subject may vary, such as ranging from about 0.0001 mg/day to about 10,000 mg/day, such as from about 0.001 mg/day to about 9000 mg/day, such as from 0.01 mg/day to about 8000 mg/day, such as from about 0.1 mg/day to about 7000 mg/day, such as from about 1 mg/day to about 6000 mg/day, including from about 5 mg/day to about 5000 mg/day. Each dosage of the compound or pharmaceutically acceptable salt administered to the subject may vary ranging from about 1 mg/kg to about 1000 mg/kg, such as from about 2 mg/kg to about 900 mg/kg, such as from about 3 mg/kg to about 800 mg/kg, such as from about 4 mg/kg to about 700 mg/kg, such as from 5 mg/kg to about 600 mg/kg, such as from 6 mg/kg to about 500 mg/kg, such as from 7 mg/kg to about 400 mg/kg, such as from about 8 mg/kg to about 300 mg/kg, such as from about 9 mg/kg to about 200 mg/kg and including from about 10 mg/kg to about 100 mg/kg.

In certain embodiments, protocols may include multiple dosage intervals. By “multiple dosage intervals” is meant that two or more dosages of the compound is administered to the subject in a sequential manner. In practicing methods of the present disclosure, treatment regimens may include two or more dosage intervals, such as three or more dosage intervals, such as four or more dosage intervals, such as five or more dosage intervals, including ten or more dosage intervals. The duration between dosage intervals in a multiple dosage interval treatment protocol may vary, depending on the physiology of the subject or by the treatment protocol as determined by a health care professional. For example, the duration between dosage intervals in a multiple dosage treatment protocol may be predetermined and follow at regular intervals. As such, the time between dosage intervals may vary and may be 1 day or longer, such as 2 days or longer, such as 4 days or longer, such as 6 days or longer, such as 8 days or longer, such as 12 days or longer, such as 16 days or longer and including 24 days or longer. In certain embodiments, multiple dosage interval protocols provide for a time between dosage intervals of 1 week or longer, such as 2 weeks or longer, such as 3 weeks or longer, such as 4 weeks or longer, such as 5 weeks or longer, including 6 weeks or longer. The cycles of drug administration may be repeated for 1, 2, 3, 4, 5, 6, 7, 8 or more than 8 dosage cycles.

In certain embodiments, compounds of the present disclosure can be administered prior to, concurrent with, or subsequent to other therapeutic agents for treating carbon monoxide exposure or poisoning. If provided at the same time as another therapeutic agent, the present compounds may be administered in the same or in a different composition. Thus, the compounds of interest and other therapeutic agents can be administered to the subject by way of concurrent therapy. By “concurrent therapy” is intended administration to a subject such that the therapeutic effect of the combination of the substances is caused in the subject undergoing therapy.

Where the compounds of the present disclosure is administered concurrently with a second therapeutic agent to treat carbon monoxide exposure or poisoning, the weight ratio of the subject compound to second therapeutic agent may range from 1:2 and 1:2.5; 1:2.5 and 1:3; 1:3 and 1:3.5 1:3.5 and 1:4; 1:4 and 1:4.5; 1:4.5 and 1:5; 1:5 and 1:10; and 1:10 and 1:25 or a range thereof. For example, the weight ratio of the subject compound to second therapeutic agent may range between 1:1 and 1:5; 1:5 and 1:10; 1:10 and 1:15; or 1:15 and 1:25. Alternatively, the weight ratio of the second therapeutic agent to the subject compound ranges between 2:1 and 2.5:1; 2.5:1 and 3:1; 3:1 and 3.5:1; 3.5:1 and 4:1; 4:1 and 4.5:1; 4.5:1 and 5:1; 5:1 and 10:1; and 10:1 and 25:1 or a range thereof. For example, the ratio of the second therapeutic agent the subject compound may range between 1:1 and 5:1; 5:1 and 10:1; 10:1 and 15:1; or 15:1 and 25:1.

Aspects, including embodiments, of the subject matter described herein may be beneficial alone or in combination, with one or more other aspects or embodiments. Without limiting the description, certain non-limiting aspects of the disclosure numbered 1-24 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
1. A compound of formula I:

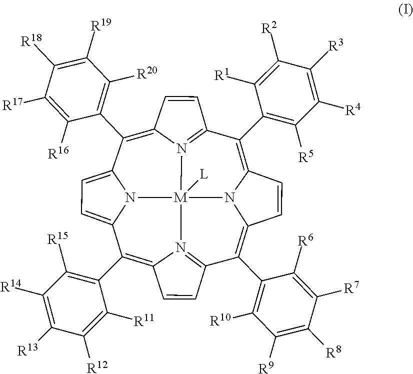

wherein:

R², R⁴, R⁷, R⁹, R¹², R¹⁴, R¹⁷ and R¹⁹ are each independently selected from hydrogen, hydroxy, alkoxy, amine, cyano, thiol, halogen, alkyl, substituted alkyl, haloalkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;

R¹, R⁵, R⁶, R¹⁰, R¹¹, R¹⁵, R¹⁶ and R²⁰ are each independently selected from substituted alkyl, haloalkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;

R³, R⁸, R¹³ and R¹⁸ are each independently a water soluble group;

M is a metal; and

L is a ligand,

or a salt, solvate or hydrate thereof.
2. The compound according to 1, wherein M is a metal selected from the group consisting of Ti, Cr, Fe, Co, Ni, Cu, Zn, Mn, Sn, Pb, Mo, Ru, Rh, Pd, Cd, Pt, Ir, and Os.
3. The compound according to 2, wherein M is Fe.
4. The compound according to any one of 1-3, wherein R², R⁴, R⁷, R⁹, R¹², R¹⁴, R¹⁷ and R¹⁹ are each independently selected from hydrogen, hydroxy, alkoxy, amine, cyano, thiol, halogen, alkyl and substituted alkyl.
5. The compound according to 4, wherein R², R⁴, R⁷, R⁹, R¹², R¹⁴, R¹⁷ and R¹⁹ are each hydrogen.
6. The compound according to any one of 1-5, wherein R¹, R⁵, R⁶, R¹⁰, R¹¹, R¹⁵, R¹⁶ and R²⁰ are each independently selected from cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl.
7. The compound according to 6, wherein R¹, R⁵, R⁶, R¹⁰, R¹¹, R¹⁵, R¹⁶ and R²⁰ are each selected from aryl, substituted aryl, arylalkyl, substituted arylalkyl, heteroaryl, substituted heteroaryl, heteroarylalkyl, and substituted heteroarylalkyl.
8. The compound according to 6, wherein R¹, R⁵, R⁶, R¹⁰, R¹¹, R¹⁵, R¹⁶ and R²⁰ are each an aryl group.
9. The compound according to 8, wherein R¹, R⁵, R⁶, R¹⁰, R¹¹, R¹⁵, R¹⁶ and R²⁰ are each independently selected from the group consisting of phenyl, 4-methylphenyl, 4-propylphenyl, 4-tertbutylphenyl, 3,5-dimethylphenyl, and 3,5-dichlorophenyl.
10. The compound according to 8, wherein R¹, R⁵, R⁶, R¹⁰, R¹¹, R¹⁵, R¹⁶ and R²⁰ are each phenyl.
11. The compound according to 6, wherein R¹, R⁵, R⁶, R¹⁰, R¹¹, R¹⁵, R¹⁶ and R²⁰ are each independently selected from the group consisting of:

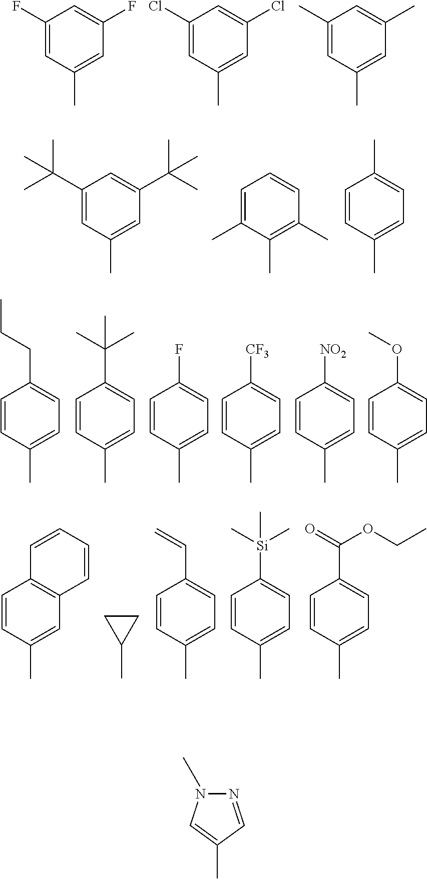

12. The compound according to any one of 1-11, wherein R³, R⁸, R¹³ and R¹⁸ are each independently the water soluble group is selected from trimethylsilyl, sulfonate, carboxylate, ammonium, trialkylammonium, pyridinium, N-alkylpyridinium, or poly (ethylene glycol).
13. The compound according to any one of 1-12, wherein L is hydroxy.
14. The compound according to any one of 1-12, wherein the compound is a salt.
15. The compound according to 14, wherein the compound is a tetrasodium salt.
16. The compound according to any one of 1-15, wherein the compound is sodium 5,10,15,20-tetrakis (2,6-diphenyl-4-(sulfonate)phenyl)porphyrinatohydroxoiron(III):

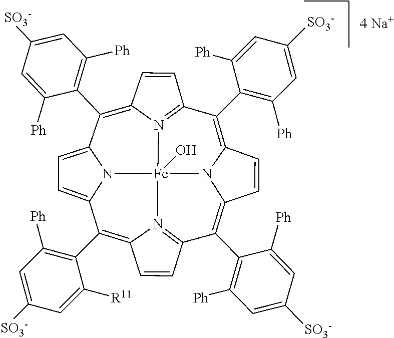

17. The compound according to any one of 1-16, wherein the compound is formed from contacting the sodium 5,10,15,20-tetrakis (2,6-diphenyl-4-(sulfonate) phenyl) porphyrinatohydroxoiron(III) with a reducing agent.
18. The compound according to 17, wherein the reducing agent is a reductant selected from the group consisting of alkylthiols, substituted alkylthiols, arylthiols, substituted arylthiols, thiol-bearing amino acids and thiol-bearing peptides.
19. The compound according to 18, wherein the reducing agent is sodium dithionite.
20 The compound according to any one of 1-19, wherein the compound is selected from:

5,10,15,20-tetrakis(2,6-diphenyl-4-(trimethylsilyl)phenyl)porphyrin;

5,10,15,20-tetrakis(2,6-di(3,5-difluoro)phenyl-4-(trimethylsilyl)phenyl)porphyrin;

5,10,15,20-tetrakis(2,6-di(3,5-dichloro)phenyl-4-(trimethylsilyl)phenyl)porphyrin;

5,10,15,20-tetrakis(2,6-di(3,5-dimethyl)phenyl-4-(trimethylsilyl)phenyl)porphyrin;

5,10,15,20-tetrakis(2,6-di(4-methyl)phenyl-4-(trimethylsilyl)phenyl)porphyrin;

5,10,15,20-tetrakis(2,6-di(4-n-propyl)phenyl-4-(trimethylsilyl)phenyl)porphyrin;

5,10,15,20-tetrakis(2,6-di(4-tert-butyl)phenyl-4-(trimethylsilyl)phenyl)porphyrin;

5,10, 15,20-tetrakis(2,6-di(4-fluoro)phenyl-4-(trimethylsilyl)phenyl)porphyrin;

5,10,15,20-tetrakis(2,6-di(4-trifluoromethyl)phenyl-4-(trimethylsilyl)phenyl)porphyrin;

5,10,15,20-tetrakis(2,6-di(4-nitro)phenyl-4-(trimethylsilyl)phenyl)porphyrin;

5,10,15,20-tetrakis(2,6-di(4-methoxy)phenyl-4-(trimethylsilyl)phenyl)porphyrin;

5,10,15,20-tetrakis(2,6-di(2-napthyl)phenyl-4-(trimethylsilyl)phenyl)porphyrin;

5,10,15,20-tetrakis(2,6-dicyclopropyl-4-(trimethylsilyl)phenyl)porphyrin;

5,10,15,20-tetrakis(2,6-di(4-vinyl)phenyl-4-(trimethylsilyl)phenyl)porphyrin;

5,10,15,20-tetrakis(2,6-di(4-trimethylsilyl)phenyl-4-(trimethylsilyl)phenyl)porphyrin;

5,10,15,20-tetrakis(2,6-di(4-ethoxycarbonyl)phenyl-4-(trimethylsilyl)phenyl)porphyrin;

5,10,15,20-tetrakis(2,6-di(N-methylpyrrazolyl)phenyl-4-(trimethylsilyl)phenyl)porphyrin;

5,10,15,20-tetrakis(2,6-diphenyl-4-(sulfonato)phenyl)porphyrin;

5,10,15,20-tetrakis(2,6-di(3,5-difluoro)phenyl-4-(sulfonato)phenyl)porphyrin;

5,10,15,20-tetrakis(2,6-diphenyl-4-(trimethylsilyl)phenyl)porphyrinatoaquazinc(II);

5,10,15,20-tetrakis(2,6-diphenyl-4-(trimethylsilyl)phenyl)porphyrinatocopper(II);

5,10,15,20-tetrakis(2,6-diphenyl-4-(trimethylsilyl)phenyl)porphyrinatopalladium(II);

5,10,15,20-tetrakis(2,6-diphenyl-4-(trimethylsilyl)phenyl)porphyrinatocobalt(II);

5,10,15,20-tetrakis(2,6-diphenyl-4-(trimethylsilyl)phenyl)porphyrinatochloroiron(III); and

5,10,15,20-tetrakis(2,6-di(3,5-dimethyl)phenyl-4-(trimethylsilyl)phenyl)porphyrinatochloroiron(III)
21. A composition comprising:

a compound according to any one of 1-20; and

a pharmaceutically acceptable excipient.
22 A method for sequestering carbon monoxide, the method comprising contacting a composition comprising carbon monoxide with a compound according to any one of 1-20.
23. The method according to any one of 1-22, wherein the method comprises contacting a composition comprising red blood cells with a compound according to any one of claims 1-18, wherein contacting the compound with the composition is sufficient to sequester carbon monoxide from the red blood cells.
24. A method for treating a subject exposed to carbon monoxide, the method comprising administering to the subject a therapeutically effective amount of a compound according to any one of claims 1-20.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for.

### Example 1

### Experimental Methods

All reactions were performed under N₂ unless otherwise stated. Glassware was oven dried prior to use. All solvents and reagents are commercially available and used as received unless stated otherwise. Pyrrole was distilled under N₂ and 1,3,5-tribromobenzene was purified by silica gel chromatography (eluted with hexanes). THF, diethyl ether, and chloroform were dried using 3-Å molecular sieves. For the purification of 6, an Isolera Prime Biotage fitted with a Sfär C18 column was employed. Analytical HPLC was performed on a Shimadzu Prominence-I LC-2030 Plus fitted with a Shimadzu Nexcol C18 5 μm column (50×3.0 mm). CDCl₃ was purchased from Cambridge Isotope Laboratories and used as received. ¹H, ¹³C{¹H}, and ²⁹Si{¹H} NMR spectra were recorded on a Bruker Avance III HD 500 NMR spectrometer equipped with a multinuclear Smart Probe. Signals in the ¹H, ¹³C, and ²⁹Si NMR spectra are reported in ppm as chemical shifts from tetramethylsilane and were referenced using the CHCl₃ (¹H, 7.26 ppm), DMSO-d6 (¹H, 2.50 ppm), or CDCl₃ (¹³C, 77.0 ppm) solvent signals or TMS in CDCl₃ (²⁹Si, 0.0 ppm). Glass background was removed from the ²⁹Si NMR spectra via backwards linear prediction of the first 100 points of the FID. UV-visible absorption spectra were measured on a Shimadzu UV-2401PC dual-beam spectrophotometer. IR spectra were recorded on a PerkinElmer Spectrum One FT-IR spectrometer. Mass spectra were obtained using a ThermoFisher LTQ Orbitrap Velos Pro. Elemental analysis was performed by Midwest Microlabs (Indianapolis, IN) using an Exeter CE440 analyzer. Melting point data were collected by an electrothermal Mel-Temp apparatus with a Fluke 52 II thermocouple probe and temperatures are uncorrected. Solution phase magnetic moments were measured using a modified Evans method.

Synthesis of (3,5-dibromophenyl)trimethylsilane (1)

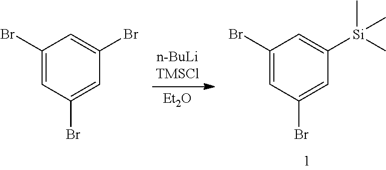

1,3,5-Tribromobenzene (13 g, 41.8 mmol) was dissolved in Et₂O (250 mL, 0.17 M) and sparged with N₂ for 10 min. This solution was cooled to −78° C. n-BuLi (17.56 mL, 43.89 mmol) was added in a dropwise manner over 30 min using a syringe pump. The reaction was allowed to stir at −78° C. for an additional 30 min. Chlorotrimethylsilane (5.8 mL, 45.98 mmol) was added in a dropwise manner over 10 min. The solution was warmed to 0° C. over approximately 20 min. The 0° C. reaction mixture was filtered through a pad of silica, which was then washed with ether. The filtrates were combined and solvent was removed under reduced pressure to give crude (3,5-dibromophenyl) trimethylsilane as a yellow oil (12.457 g, 97% yield) that solidified upon standing at room temperature. This crude product was dissolved in hexanes and passed through a pad of silica. Solvent was removed from the eluent under reduced pressure to give an off-white solid (11.825 g 93% yield). Recrystallization from cold ethanol afforded the pure product as colorless needles (9.925 g, 78% yield). ¹H NMR (500 MHZ, CDCl₃) δ7.66-7.62 (m, 1H), 7.51 (s, 2H), 0.27 (s, 9H); ¹³C{¹H} NMR (126 MHz, CDCl₃) δ146.19, 134.62, 134.32, 123.34, −1.21; ²⁹Si{¹H} NMR (99 MHz, CDCl₃) δ−2.16; Melting point: 41.2° C.; Anal. Calcd for C₉H₁₂Br₂Si: C, 35.09; H, 3.93. Found: C, 34.61; H, 3.65.

Synthesis of 2,6-dibromo-4-trimethylsilylbenzaldehyde (2)

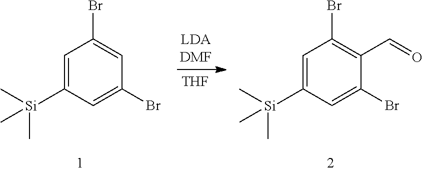

(3,5-Dibromophenyl) trimethylsilane (7 g, 22.9 mmol) was dissolved in THF (225 mL, 0.1 M), cooled to −78° C., and sparged with N₂ for 10 min. Lithium diisopropylamide (2.0 M in THF/heptane/ethylbenzene, 45.8 mL, 91.8 mmol) was added in a dropwise manner over 30 min such that the reaction temperature, monitored with a thermocouple probe, did not exceed −75° C. The reaction was stirred at this temperature for 1.5 h. DMF (7.9 mL, 103.28 mmol) was added in a dropwise manner and the reaction was stirred for an additional 1.5 h. Aqueous 1 M H₂SO₄ (100 mL) was added and the product was extracted with ether (100 mL). The organic layer was dried over sodium sulfate and filtered. The crude product was dry-loaded onto silica gel and purified by column chromatography (silica gel, hexanes: ether 95:5) yielding 2,6-dibromo-4-trimethylsilylbenzaldehyde as a pale-yellow oil that solidified while drying under vacuum (6.20 g, 80% yield). This crude product was then recrystallized from hot ethanol and the pale-yellow needles were collected by filtration. Two crops were collected (first crop 4.562 g, second crop 1.072 g, 73% combined yield). ¹H NMR (500 MHZ, CDCl₃)δ10.25 (s, 1H), 7.69 (s, 2H), 0.31 (s, 9H); ¹³C{¹H} NMR (126 MHz, CDCl₃) δ191.39, 150.50, 138.11, 132.71, 124.81, −1.41; Melting point: 89.8° C.; Anal. Calcd for C₁₀H₁₂Br₂OSi: C, 35.74; H, 3.60. Found: C, 35.28; H, 3.53.

Synthesis of 5,10,15,20-tetrakis(2,6-dibromo-4-(trimethylsilyl)phenyl)porphyrin (3)

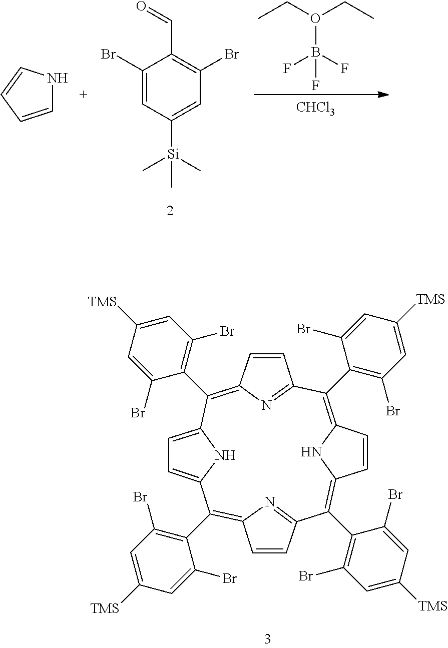

A mixture of 2,6-dibromo-4-trimethylsilylbenzaldehyde (1.754 g, 5.22 mmol), and pyrrole (350 mg, 5.22 mmol) in CHCl₃ (350 mL) and EtOH (0.2 mL) was added to a 1 L oven-dried round bottom flask fitted with magnetic stirrer. The reaction mixture was sparged with N₂ for 20 min, followed by the addition of BF₃ etherate (185 mg, 1.3 mmol, 0.16 mL). The solution became yellow and slowly darkened to wine red. After stirring the solution for 16 h in the dark at room temperature, 2,3-dichloro-5,6-dicyano-1,4-benzoquinone (2.371 g, 10.4 mmol) was added in one portion, turning the solution black. This solution was allowed to stir for 2 h. The crude mixture was filtered through a pad of silica gel, which was then washed with chloroform. The combined filtrates yielded a purple solid after concentration under reduced pressure. This solid was washed with acetonitrile to give 5,10, 15,20-tetrakis(2,6-dibromo-4-(trimethylsilyl) phenyl) porphyrin as a purple solid after drying (957 mg, 48% yield). X-ray quality crystals were grown by layering MeCN over the product in CHCl₃ to give purple plates. ¹H NMR (500 MHz, CDCl₃) δ8.65 (s, 8H), 8.08 (s, 8H), 0.53 (s, 36H), −2.42 (s, 2H); ¹³C{¹H} NMR (126 MHz, CDCl₃) δ146.06, 142.97, 136.01, 128.76, 118.76, −0.89; Melting point: >400° C.; Anal. Calcd for C₅₆H₅₄Br₈N₄Si₄: C, 43.83; H, 3.55; N, 3.65. Found: C, 43.36; H, 3.52; N, 3.60; UV/Vis (CHCl₃) λ_{abs} (log ε): 406 (sh), 424 (4.64), 518 (3.35), 593 (2.86).

Synthesis of 5,10,15,20-tetrakis(2,6-diphenyl-4-(trimethylsilyl)phenyl)porphyrin (4)

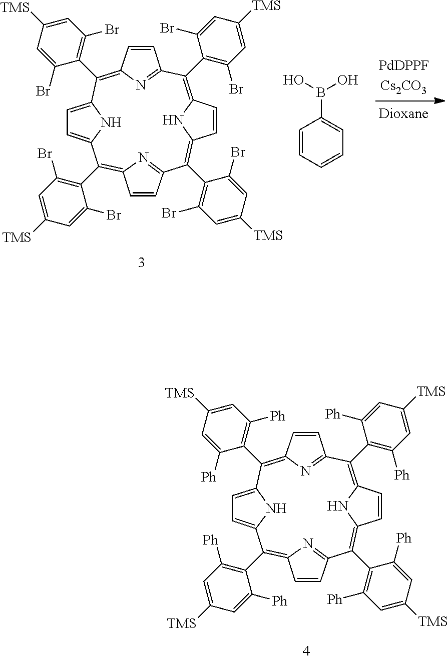 [SMILES: C[Si](C)(C)c1cc(Br)c(-c2c3nc(c(-c4c(Br)cc([Si](C)(C)C)cc4Br)c4ccc([nH]4)c(-c4c(Br)cc([Si](C)(C)C)cc4Br)c4nc(c(-c5c(Br)cc([Si](C)(C)C)cc5Br)c5ccc2[nH]5)C=C4)C=C3)c(Br)c1 ; OB(O)c1ccccc1 ; C[Si](C)(C)c1cc(-c2ccccc2)c(-c2c3nc(c(-c4c(-c5ccccc5)cc([Si](C)(C)C)cc4-c4ccccc4)c4ccc([nH]4)c(-c4c(-c5ccccc5)cc([Si](C)(C)C)cc4-c4ccccc4)c4nc(c(-c5c(-c6ccccc6)cc([Si](C)(C)C)cc5-c5ccccc5)c5ccc2[nH]5)C=C4)C=C3)c(-c2ccccc2)c1]

5,10,15,20-Tetrakis 2,6-dibromo-4-(trimethylsilyl)phenyl)porphyrin (300 mg, 0.1967 mmol), [1,1′-bis(diphenylphosphino)ferrocene]dichloropalladium(II) (144 mg, 0.1967 mmol), phenylboronic acid (576 mg, 4.7213 mmol), and cesium carbonate (2.061 g, 6.251 mmol) were dissolved in a mixture of dioxane (20 mL) and H₂O (1 mL). The solution was sparged with N₂ for 5 min. The reaction was sealed with a septum and stirred at 100° C. for 14 h. The reaction crude mixture was stripped of solvent under reduced pressure. The residue was taken up in chloroform (50 mL) and passed through a pad of silica gel. The filtrate was dried to give a purple solid that was washed with acetonitrile. The washed solid was dissolved in chloroform and dry loaded onto silica gel. The product was purified by column chromatography (silica, hexanes: chloroform 1:1). The eluted product was concentrated to give 4 as a purple solid (256 mg, 86% yield). X-ray quality crystals were grown by layering MeCN over the product in CHCl₃to give purple plates. ¹H NMR (500 MHz, CDCl₃) δ8.38 (s, 8H), 7.77 (s, 8H), 6.56 (d, J=7.7 Hz, 16H), 6.40 (t, J=7.2 Hz, 8H), 6.22 (t, J=7.4 Hz, 16H), 0.51 (s, 36H), −3.40 (s, 2H); ¹³C{¹H} NMR (126 MHz, CDCl₃) δ144.80, 142.44, 140.75, 139.39, 133.66, 129.44, 126.67, 125.22, 116.12, −0.62; ²⁹Si {¹H} NMR (99 MHz, CDCl₃) δ−3.44; Melting point: >400° C.; HRMS (MALDI) m/z: [M+H]⁺ Calcd for C₁₀₄H₉₅N₄Si₄⁺1512.6662; Found 1512.6650; UV/Vis (CHCl₃) λ_{abs} (log ε): 419 (sh), 439 (4.54), 495 (2.62), 533 (3.17), 570 (2.93), 610 (2.79), 670 nm 5 (2.34).

Synthesis of 5,10,15,20-tetrakis(2,6-diphenyl-4-(trimethylsilyl)phenyl)porphyrinatohydroxoiron(III) (5)

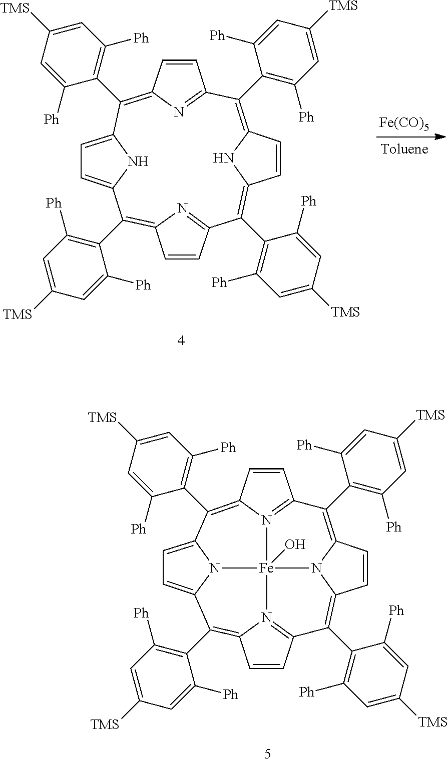

5,10,15,20-Tetrakis (2,6-diphenyl-4-(trimethylsilyl)phenyl)porphyrin (200 mg, 0.1325 mmol), iron pentacarbonyl (2.589 g, 13.2 mmol, 1.786 mL), and iodine (101 mg, 0.397 mmol) were dissolved in toluene (30 mL) and refluxed for 5 h under N₂. This solution was then refluxed another 1 h under ambient conditions. The solution was concentrated under reduced pressure. The residue was taken up in CHCl₃ and filtered through a pad of Celite. The filtrate was added to a separatory funnel and washed with 1 M NaOH_{(aq)}. The organic layer was dried with anhydrous sodium sulfate, filtered, and concentrated under reduced pressure to give 5 as a green solid (183 mg, 88% yield). Weakly diffracting dark purple plates were grown by layering MeCN over a solution of the product in CHCl₃. ¹H NMR (500 MHZ, CDCl₃; paramagnetic) δ82.12 (β-pyrrole); Melting point: >400° C.; HRMS (MALDI) m/z: [M−OH]⁺ Calcd for C₁₀₄H₉₂FeN₄Si₄⁺1565.5777; Found 1565.5786. [M+H]⁺ Calcd for C₁₀₄H₉₄FeN₄Osi₄⁺1583.5883; Found 1583.5887; UV/Vis (CHCl₃) λabs (log ε): 360 (sh), 379 (4.52), 443 (5.14), 524 (4.07), 596 (sh); μ_{eff} (Evans', CDCl₃): 5.49 μ_{B}

Synthesis of Sodium 5,10,15,20-tetrakis(2,6-diphenyl-4-(sulfonate)phenyl)porphyrinatohydroxoiron(III) (6)

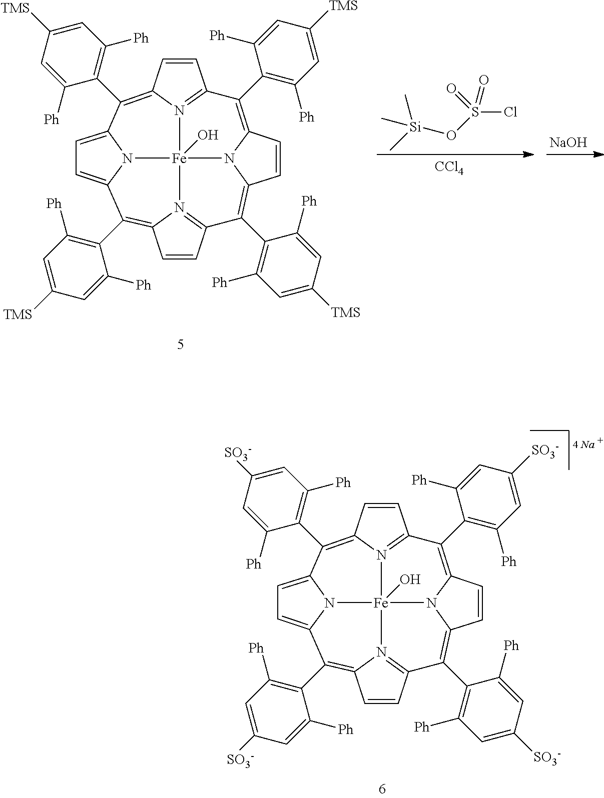

Compound 5 (50 mg, 0.0316 mmol) was dissolved in CCl₄ (4 mL). To this solution was added trimethylsilyl chlorosulfonate (72 mg, 0.38 mmol, 0.058 mL). The solution was stirred at reflux for 60 min under N₂. After cooling to room temperature, 1 M NaOH (aq) (5 mL) was added and the reaction was stirred vigorously for 10 min. This solution was diluted with 50 mL of DI water, washed with 50 mL of chloroform, and stripped of solvent under reduced pressure to give a green solid. This solid was dry-loaded onto C18-functionalized silica gel and eluted across 25 g of stationary phase (6.35 cm) with a gradient of H₂O/MeCN containing 0.01% TFA (5-95% MeCN over 15 min). The first colored fraction to elute from the column was collected and dialyzed against DI water for 3 d (changing dialysate every 12 h). The retentate was lyophilized yielding the tetrasodium salt 6 as a dark purple/black solid (22 mg 40% yield). Weakly diffracting crystals of 6 were grown from CHCl₃/DMSO. Higher-quality crystals (6_{DMSO}) were grown slowly by layering CHCl₃ over the product in DMSO. ¹H NMR (500 MHZ, CDCl₃; paramagnetic) δ80.41 (β-pyrrole); HRMS (ESI) m/z: [M−4Na−OH+H]³⁻ Calcd for C₉₂H₅₇FeN₄O₁₂S₄³⁻531.0740; Found 531.0711. [M−3Na−OH+H]²⁻ Calcd for C₉₂H₅₇FeN₄NaO₁₂S₄²⁻808.1057; Found 808.1004. [M−4Na−OH+H+MeOH]³⁻k Calcd for C₉₃H₆₁FeN₄O₁₃S₄³⁻541.7494; Found 541.7454. [M−OH−4Na]⁴⁻ Calcd for C₉₂H₅₆FeN₄O₁₂S₄⁴⁻398.3046; Found 398.3028; HPLC (H₂O/MeCN): tᵣ=1.10 min; UV/Vis (PBS) λ_{abs} (log ε): 333 (3.19), 431 (3.82), 509 (sh), 545 (sh); μ_{eff} (Evans', DMSO-d₆): 5.75 μ_{B}

### In Situ Reduction of 6 to 7

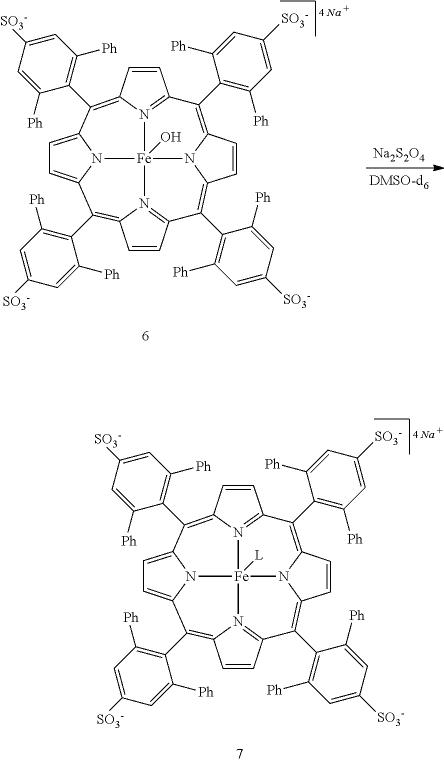

For NMR spectroscopic characterization, compound 6 (5 mg) was dissolved in DMSO-d₆ (1 mL). To this solution was added 1 drop of D₂O. The solution was sparged with N₂ for 5 min and a ¹H NMR spectrum was acquired. Sodium dithionite (10 mg) was added to the NMR tube and the mixture was shaken and periodically sonicated for 20 min. μ_{eff} (Evans', DMSO-d₆): 2.50 μ_{B}.

For UV-vis characterization, compound 6 was dissolved in PBS and diluted to 0.02 mM. A minimal amount of sodium dithionite was added to effect reduction of 6 to 7, which resulted in an immediate change in the electronic absorption spectrum. UV/Vis (PBS) λ_{abs} (log ε): 448 (5.08), 551 (3.81), 578 (sh), 625 (3.38). Air was bubbled through the solution to remove any excess sodium dithionite as assessed by reduction in intensity of the absorption at 315 nm. Once all of the dithionite had been consumed, the quiescent solution was left open to air and electronic absorption spectra were acquired at 90 s intervals (FIG. 4)

### In Situ Preparation of 8

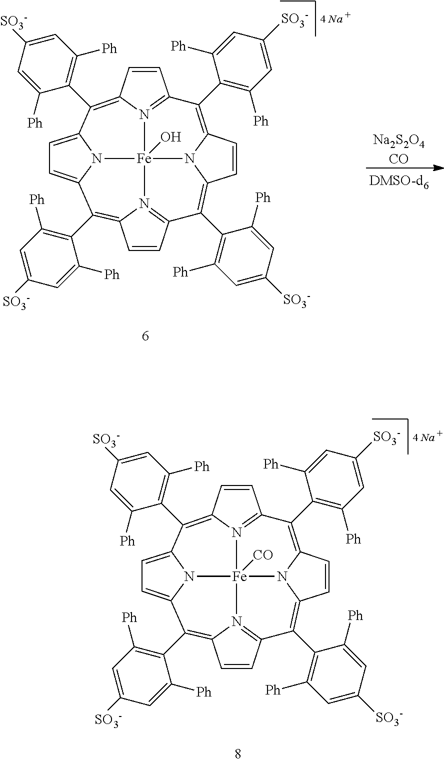

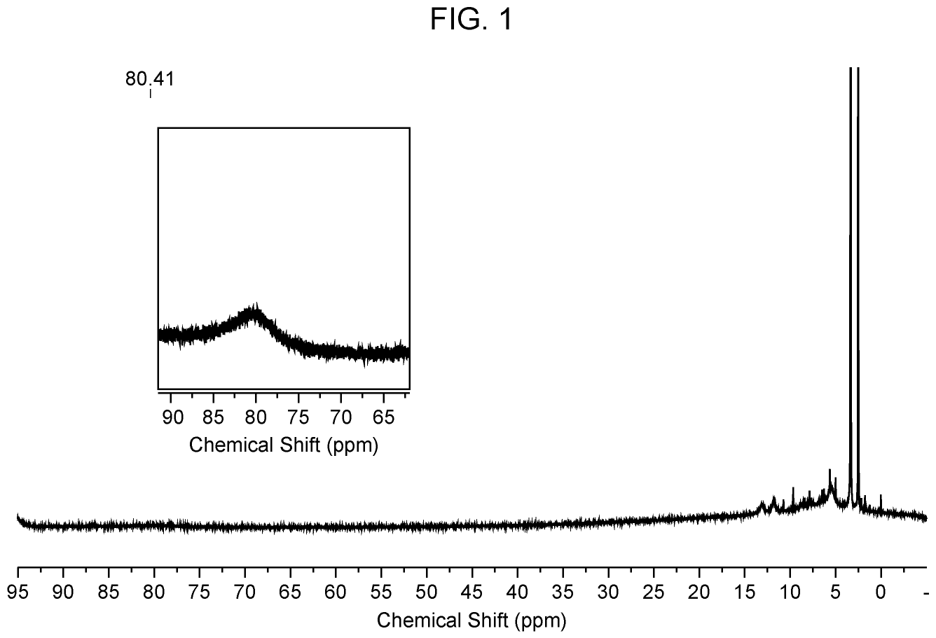

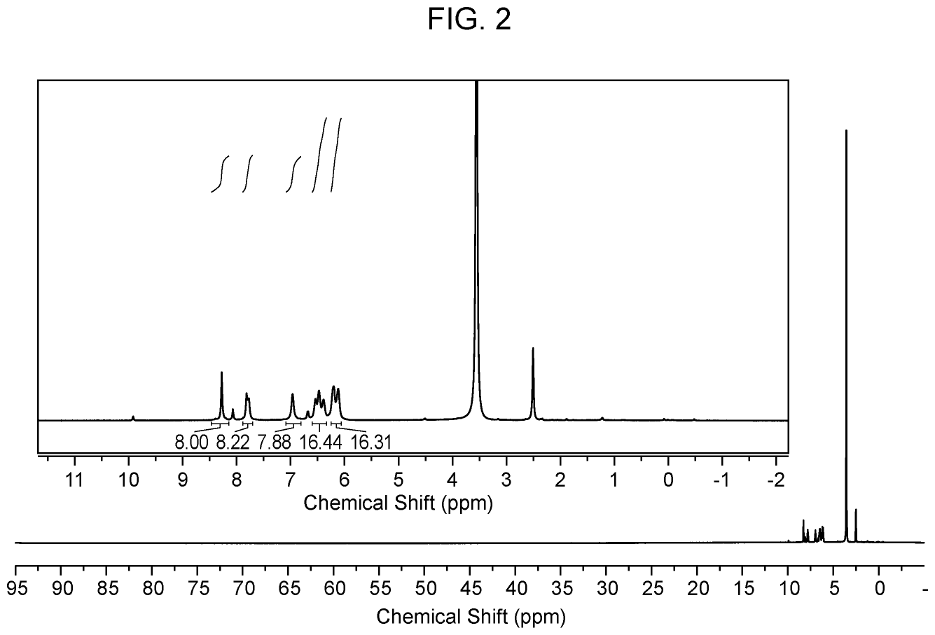

For NMR spectroscopic characterization, compound 6 (5 mg) was dissolved in DMSO-d₆ (1 mL). To this solution was added 1 drop of D₂O. The solution was sparged with N₂ for 5 min and a ¹H NMR spectrum was acquired (FIG. 1). Sodium dithionite (10 mg) was added to the NMR tube and the mixture was shaken and periodically sonicated for 20 min. Then, CO was bubbled through the solution for approximately 5 s. The NMR tube was sealed and then sonicated and shaken for 15 min (FIG. 2). ¹H NMR (500 MHz, DMSO-d₆) δ8.28 (s, 8H), 7.87-7.71 (m, 8H), 6.96 (s, 8H), 6.62-6.36 (m, 16H), 6.16 (m, 16H); μ_{eff} (Evans', DMSO-d₆): 0 μ_{B};

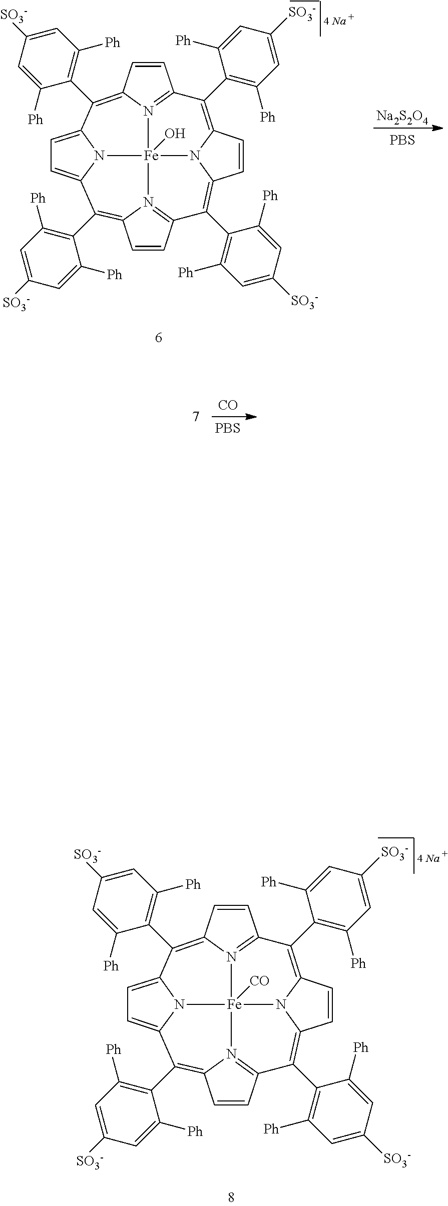

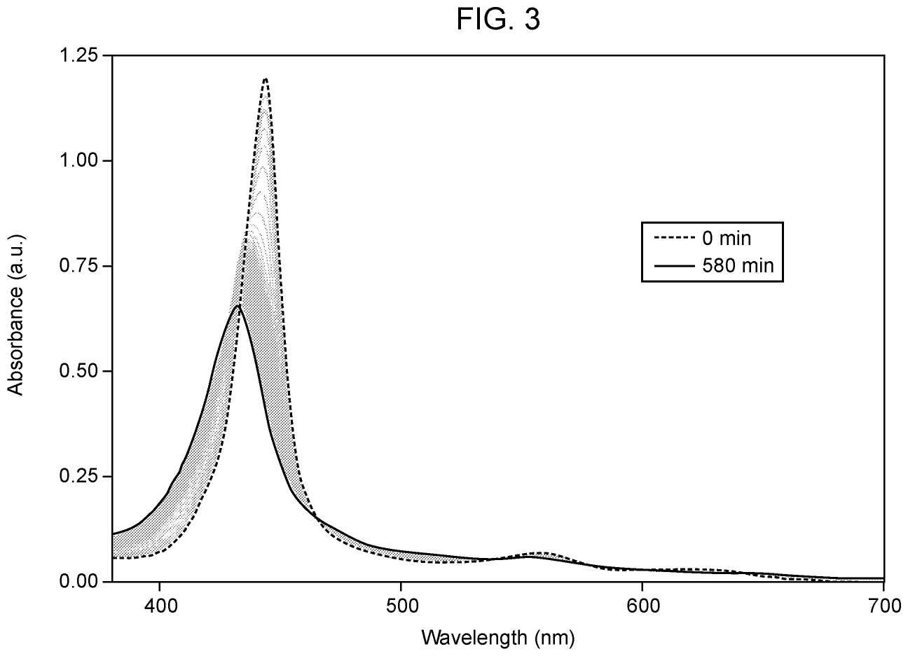

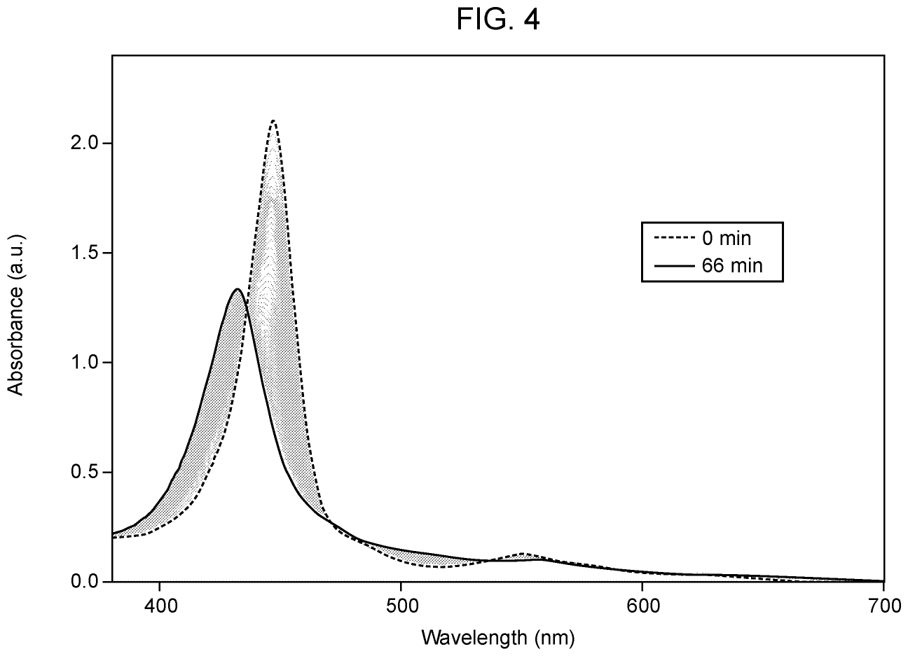

For UV-vis characterization, compound 6 was dissolved in PBS and diluted to 0.02 mM. A minimal amount of sodium dithionite was added to effect reduction of 6 to 7. CO was bubbled through for 5 seconds to generate compound 8, which resulted in an immediate change in the electronic absorption spectrum. UV/Vis (PBS) λ_{abs} (log ε): 444 (5.08), 557 (3.85), 624 (3.48). Air was bubbled through the solution to remove any excess sodium dithionite as assessed by reduction in intensity of the absorption at 315 nm. Once all of the dithionite had been consumed, the quiescent solution was left open to air and electronic absorption spectra were acquired at 600 s intervals (FIG. 3).

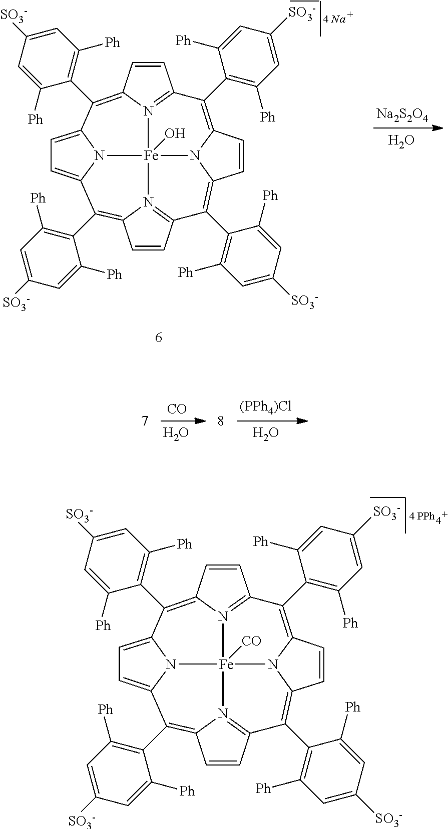

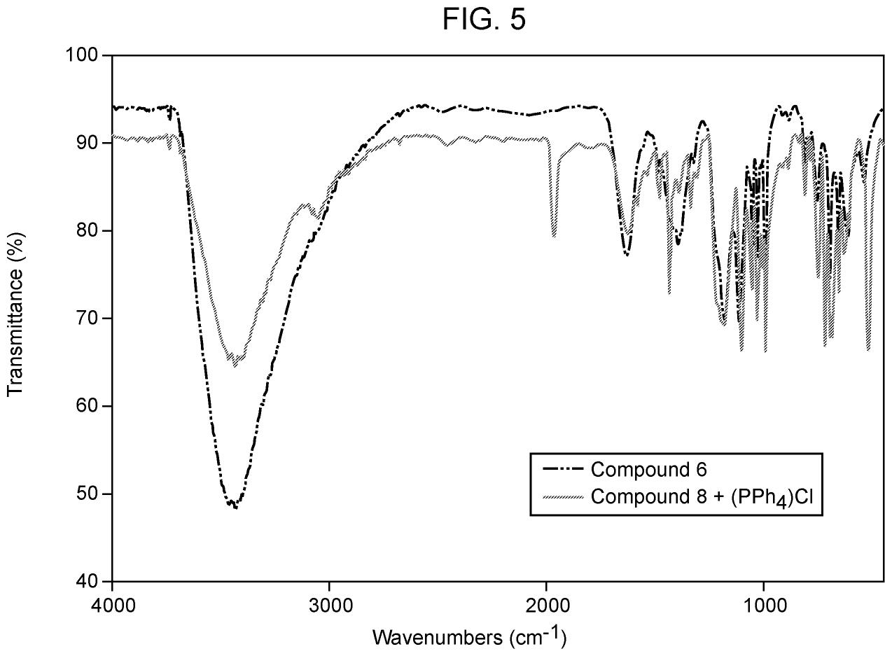

To collect IR data, compound 6 (5 mg) was dissolved in DI water (5 mL). The solutions was sparged with nitrogen and excess sodium dithionite was added (1 mg). CO was bubbled through the solution for 5 s. To this solution was added an excess of tetraphenylphosphonium chloride (5 mg). The resulting precipitate was collected, washed with DI water, and dried under a stream of N₂ for 5 min. The resulting solid was used to prepared a KBr pellet for IR spectroscopic measurement (FIG. 5).

### X-Ray Crystallography

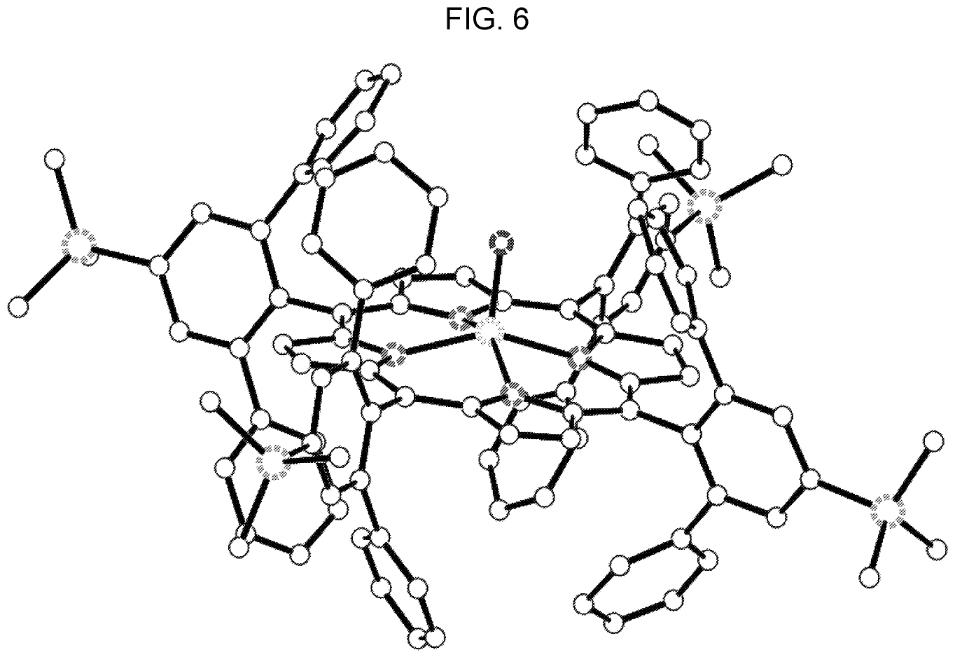

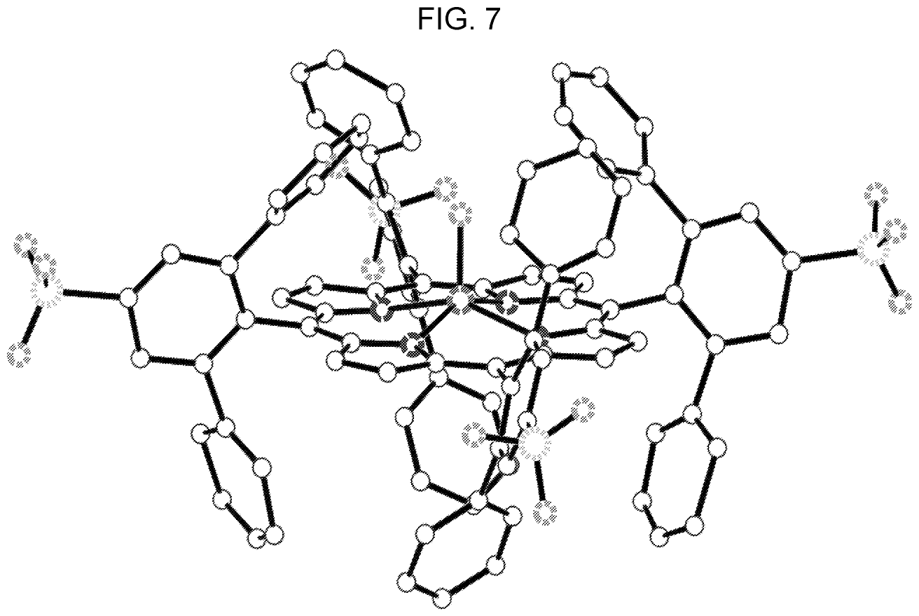

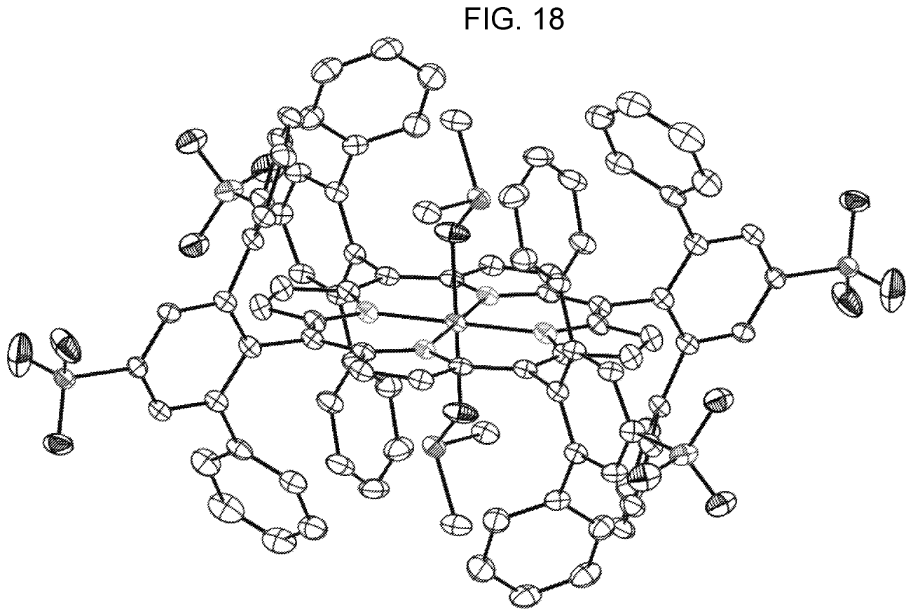

Crystals of 4.2MeCN, 5, 6, and 6_{DMSO}.4DCM were grown as described above, selected under a microscope, loaded onto a nylon fiber loop using Paratone-N, and mounted onto a Rigaku XtaLAB Synergy-S single crystal diffractometer. Each crystal was cooled to 100 K under a stream of nitrogen. Diffraction of Cu Kα radiation from a PhotonJet-S microfocus source was detected using a HyPix-6000HE hybrid photon counting detector. Screening, indexing, data collection, and data processing were performed with CrysAlis^{Pro}. The structures were solved using SHELXT and refined using SHELXL as implemented in OLEX2 following established strategies. The contents of the unit cell of 4.2MeCN are depicted in FIG. 18. The crystals of 5 and 6 were twinned and diffracted weakly. The diffraction data allowed the proposed connectivity of iron complexes to be confirmed (FIGS. 6 and 7), but were not suitable for detailed analysis of bond metrics. Notably, in both 5 and 6, the apical ligand is located 1.9 Å from the Fe center and exhibited an electron density consistent with an O atom. For the atomic-resolution resolution crystal structures of 4.2MeCN and 6_{DMSO}.4DCM, all non-H atoms were refined anisotropically and carbon-bound H atoms were placed at calculated positions and refined with a riding model and coupled isotropic displacement parameters (1.2×U_{eq} for aryl groups and 1.5×U_{eq} for methyl groups). Refinement parameters for 4.2MeCN and 6_{DMSO}.4DCM are collected in Table 1. The unit cell parameters for 5 and 6 are collected in Table 2.

TABLE 1
Refinement Details for High-Resolution Crystal Structures

| Compound | 4•2MeCN | 6_{DMSO}•4DCM |
|---|---|---|
| Formula | C₁₀₈H₁₀₀N₆Si₄ | C₁₁₁H₉₉Cl₂₁FeN₄Na₃O₁₈S₁₀ |
| FW | 1594.29 | 2966.81 |
| T (K) | 100.0(1) | 100.0(1) |
| λ (Å) | 1.54184 | 1.54184 |
| Crystal | Triclinic | Triclinic |
| System | | |
| Space group | P1 | P1 |
| a (Å) | 13.1696(2) | 14.03520(10) |
| b (Å) | 13.5821(2) | 14.48810(10) |
| c (Å) | 16.0892(2) | 16.95090(10) |
| α (°) | 89.2370(10) | 72.6450(10) |
| β (°) | 71.1150(10) | 80.2710(10) |
| γ (°) | 61.112(2) | 88.616(2) |
| Volume | 2348.33 | 86.7770(10) |
| (Å³) | | |
| Z | 1 | 1 |
| ρ_{calc} | 1.127 | 1.519 |
| (Mg/m³) | | |
| Size (mm³) | 0.25 × 0.19 × 0.05 | 0.15 × 0.08 × 0.08 |
| θ range (°) | 2.95-67.07 | 2.77-67.08 |
| Total data | 89543 | 95102 |
| Unique data | 8389 | 11584 |
| Parameters | 540 | 831 |
| Completeness (%) | 100 | 99.9 |
| Rᵢₙₜ (%) | 4.46 | 3.48 |
| R₁ | 3.38 | 5.73 |
| (%, I > 2σ) | | |
| R₁ | 3.57 | 5.90 |
| (%, all data) | | |
| wR₂ | 8.59 | 15.81 |
| (%, I > 2σ) | | |
| wR₂ | 8.72 | 15.95 |
| (%, all data) | | |
| S | 1.041 | 1.037 |

TABLE 2
Crystallographic Parameters for Low-Resolution Crystal Structures

| Compound | 5 | 6 |
|---|---|---|
| T (K) | 100.0(1) | 100.0(1) |
| λ (Å) | 1.54184 | 1.54184 |
| Crystal | Triclinic | Tetragonal |
| System | | |
| Space group | P1̅ | P4₂/n |
| a (Å) | 16.3623(6) | 23.1879(2) |
| b (Å) | 12.8462(5) | |
| c (Å) | 23.4865(7) | 24.2311(3) |
| β (°) | 106.700(4) | |
| Volume | 4728.5(3) | 13028.5(2) |
| (Å³) | | |
| Z | 2 | 4 |

### CO Abstraction From Carboxy-Hemoglobin (COHb)

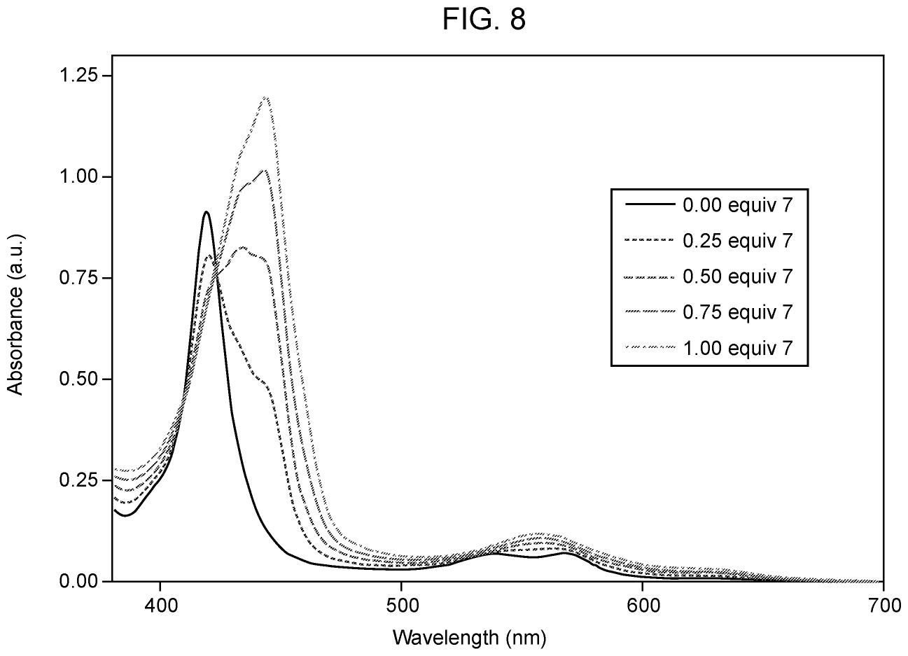

A stock solution of COHb was created by dissolving bovine Hb (5 mg) in 1 mL of PBS containing 5.7 mM sodium dithionite that had been sparged with N₂. CO was bubbled through this solution for 5 s. N₂ was slowly bubbled through this solution for 20 min to remove excess CO. Working solutions were prepared from this stock by dilution with PBS containing 5.7 mM sodium dithionite. Concentrations were determined with the mass of Hb used to prepared the stock solution and a molecular weight of 64,500 g/mol. For CO abstraction, a 2.5 μM PBS solution of COHb was prepared and titrated with a PBS solution of 6. Equivalents of 6 were calculated per heme unit of Hb (i.e., 4× molar quantity of protein). Spectra are presented in FIG. 8.

### Compound 7 Protects Hb From CO

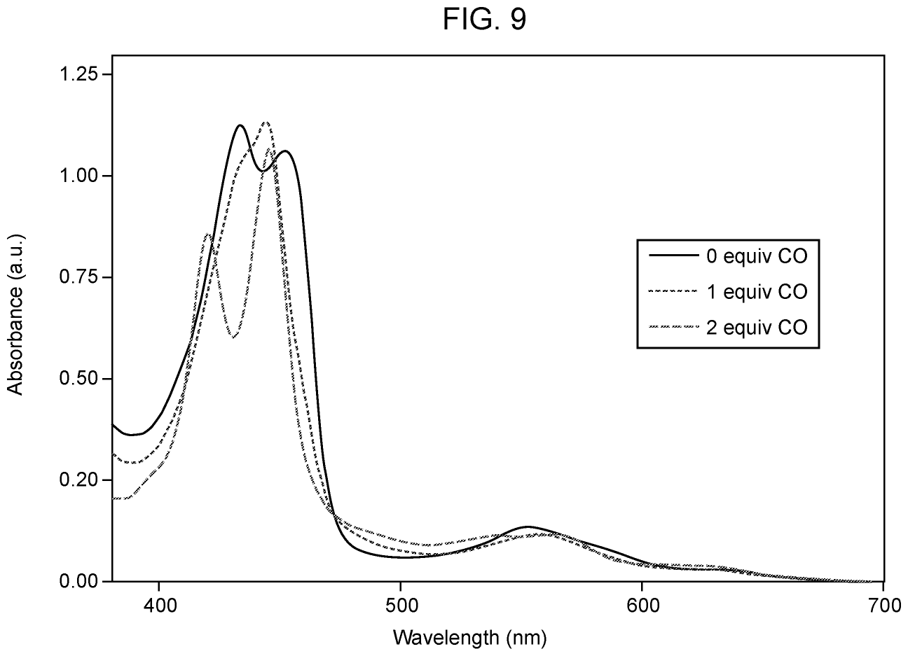

A stock solution of bovine hemoglobin was prepared by dissolving 5 mg in 1 mL of N₂-sparged PBS containing 5.7 mM sodium dithionite. Working solutions were prepared from this stock by dilution with PBS containing 5.7 mM sodium dithionite. Concentrations were determined with the mass of Hb used to prepared the stock solution and a molecular weight of 64,500 g/mol. From this stock, a working solution containing 2.5 μM Hb and 10 μM 7 (prepared from in situ reduction of 6) was prepared and titrated with CO-saturated water (approx. 1 mM CO (aq)). UV-vis spectra were acquired after addition of 1 and 2 equivalents (with respect to 7) of CO (FIG. 9).

### Hemolytic Potential of Compound 7

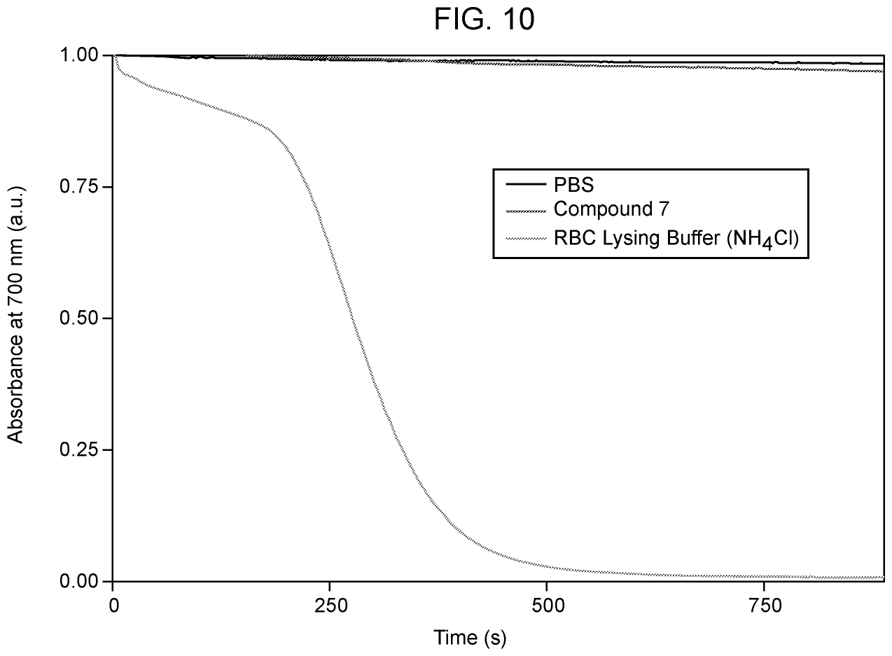

Defibrinated bovine blood (Hemostat Laboratories) was diluted with PBS containing 5.7 mM sodium dithionite. This mixture was centrifuged for 30 s at 760× g. The supernatant was discarded, and the pellet was washed with PBS containing 5.7 mM sodium dithionite. The pellet was suspended in PBS containing 5.7 mM sodium dithionite to give a suspension with A₇₀₀=1.0. An aliquot of this suspension was lysed and the absorbance at 420 nm was used to quantify the amount of COHb (ε=10^{5.63}). Based on this concentration, 1 equiv of compound 6 was added to this solution, which was reduced immediately to 7. After the addition, turbidity was monitored continuously at 700 nm. This process was repeated both in the absence of any added species (negative control) and upon addition of a RBC-lysing solution (1.5 M NH₄Cl) (FIG. 10).

### CO Abstraction From CO-Treated Red Blood Cells (RBCs)

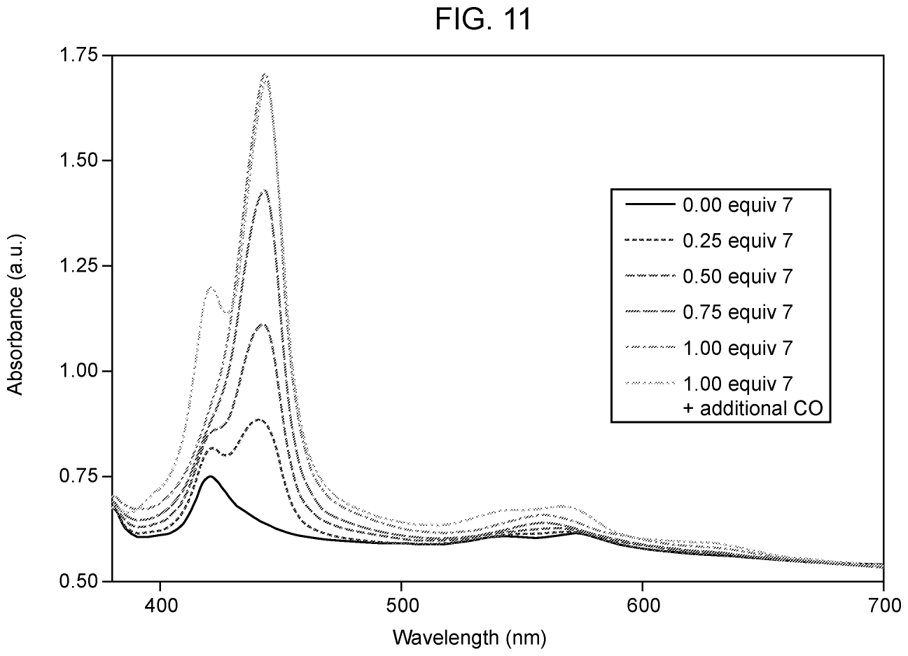

Defibrinated bovine blood (Hemostat Laboratories) was diluted with PBS containing 5.7 mM sodium dithionite. CO was bubbled through this suspension for 5 s. This mixture was centrifuged for 30 s at 760×g. The supernatant was discarded, and the pellet was washed with PBS containing 5.7 mM sodium dithionite. This washing was repeated three more times to remove excess CO. An aliquot of the stock suspension of CO-treated RBCs was added to a quartz cuvette containing 1 mL of DI water to lyse the cells. The concentration of COHb was assessed by measuring the absorbance at 420 nm (ε=10^{5.63}) of this lysate. For abstraction studies, an aliquot of the stock suspension of CO-treated RBCs was diluted to 1 mL with PBS containing 5.7 mM sodium dithionite. Compound 6, which is reduced in situ to 7, was added in increments based on the concentration of COHb determined in the lysate. A UV-vis spectrum was acquired after each addition (FIG. 11).

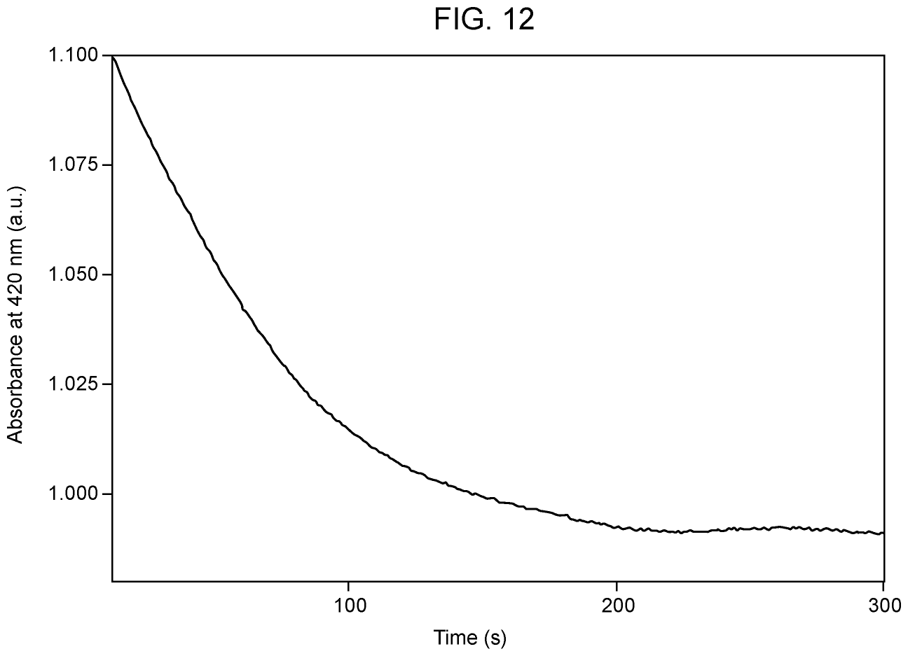

Time-Course CO Removal From CO-Treated RBCs Defibrinated bovine blood (Hemostat Laboratories) was diluted with PBS containing 5.7 mM sodium dithionite. CO was bubbled through this suspension for 5 s. This mixture was centrifuged for 30 s at 760×g. The supernatant was discarded, and the pellet was washed with PBS containing 5.7 mM sodium dithionite. This washing was repeated three more times to remove excess CO. An aliquot of the stock suspension of CO-treated RBCs was added to a quartz cuvette containing 1 mL of DI water to lyse the cells. The concentration of COHb was assessed by measuring the absorbance at 420 nm (ε=10^{5.63}) of this lysate. For time-course CO removal, an aliquot of the stock suspension of CO-treated RBCs was diluted to 1 mL with PBS containing 5.7 mM sodium dithionite. A full equivalent of compound 6 (which is reduced in situ to 7) was added, the suspension was rapidly mixed, and absorbance at 420 nm was monitored continuously (FIG. 12).

### Results

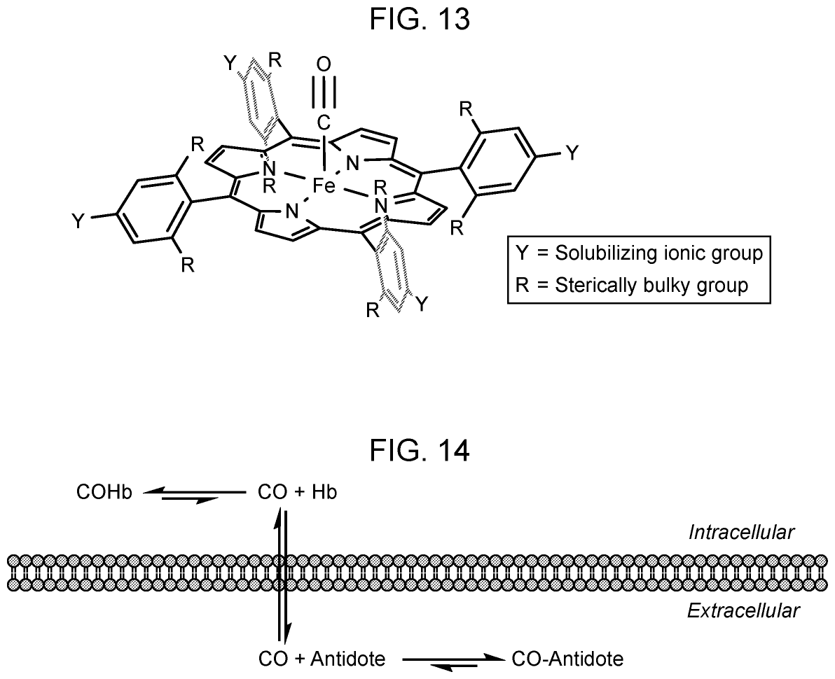

The design of the subject water soluble metal porphyrin complexes is designed based on binding of CO more strongly than O₂. Steric bulk can prevent the formation of deactivated μ-oxo species. Water soluble metal porphyrin complexes according to certain instances are symmetrically substituted bis-pocket porphyrins featuring 2,6-disubstituted meso-phenyl groups. The 4-position of the meso-phenyl unit in some instances can be functionalized with a charged group to impart water solubility (FIG. 13).

The modular nature of the framework depicted in FIG. 1, allows for readily tuning the CO-binding pocket, the electronic structure of the Fe center, and the overall physicochemical properties of the compound. The large, highly charged antidote will have limited-to-no cellular uptake by design; cellular uptake is not required because the water soluble metal porphyrin complexes will not need to interact directly with COHb to function. The thermodynamic stability of COHb does not preclude kinetic lability, which has been exploited to transfer CO between heme proteins. If the water soluble metal porphyrin complexes has a CO affinity sufficiently greater than that of Hb, transfer will proceed (FIG. 14).

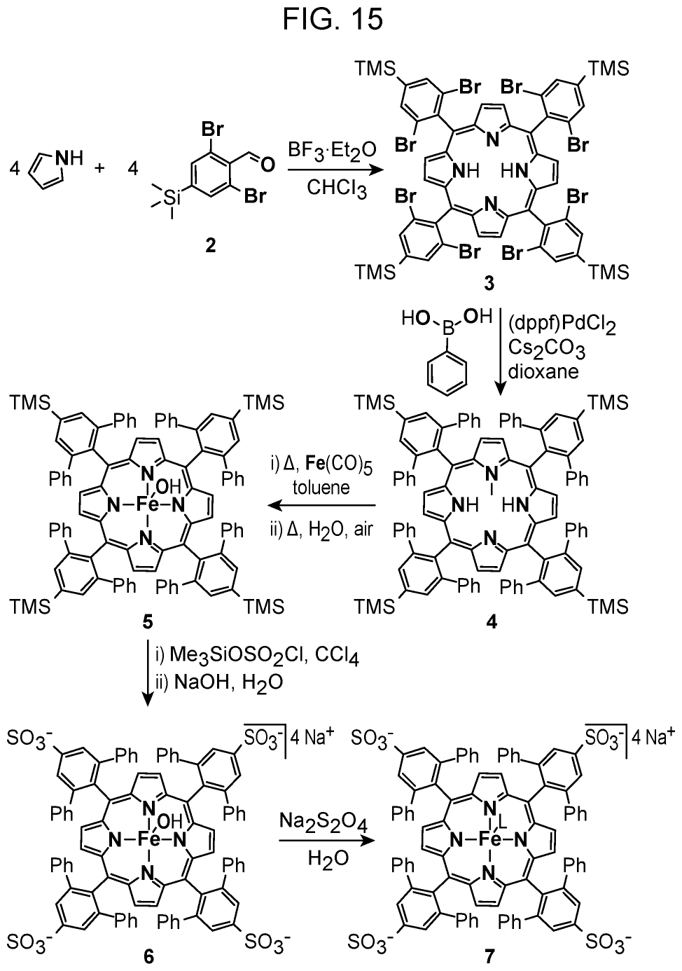

The synthesis of iron-porphyrin complex 7 with 2,6-diphenyl-4-sulfophenyl meso substituents (Scheme 1, FIG. 15). The orth o-phenyl groups prevent μ-oxo dimer formation and create a hydrophobic CO-binding pocket. The para-sulfonate groups ensure water solubility at physiological pH. The bis-pocket porphyrin with 2,4,6-triphenylphenyl meso substituents was accessed using a standard Lindsey synthesis, but macrocyclization was inhibited by the extreme steric congestion in the product; the final yield was 1%. 2,6-dibromophenyl-containing meso substituents were targeted so that steric bulk can be incorporated via Pd-catalyzed cross-coupling reactions after macrocyclization. The complications associated with harsh electrophilic aromatic sulfonation was avoided by incorporating a trimethylsilyl group that can undergo facile late-stage conversion to a sulfonyl chloride, which can then be hydrolyzed to a sulfonate.

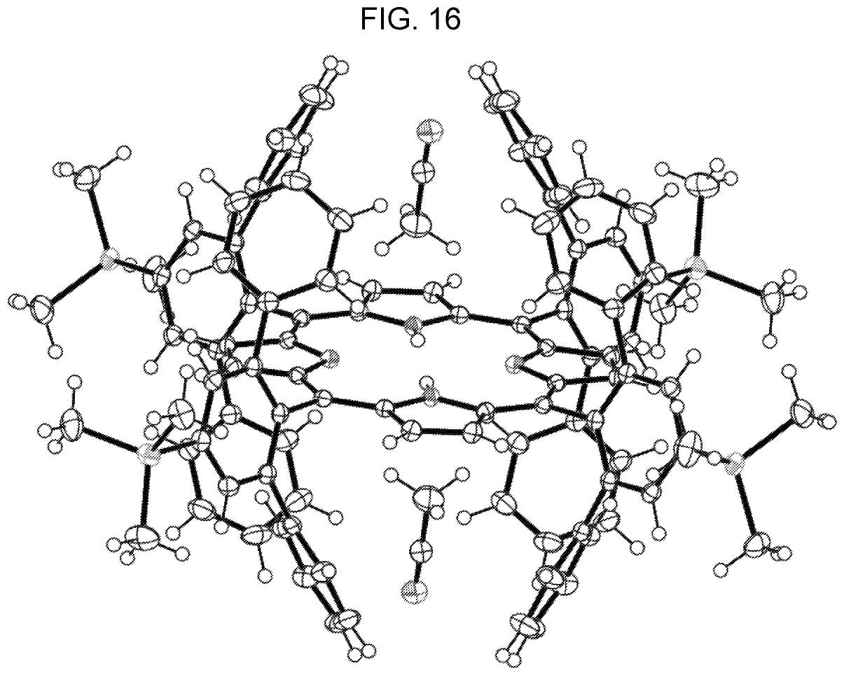

Turning to Scheme 1 in FIG. 15, silylated compound 1 was readily prepared from 1,3,5-tribromobenzene via sequential reaction with n-BuLi and Me₃SiCl. Conversion to aldehyde 2 was achieved via deprotonation with LDA and carbonylation with DMF. BF₃.Oet₂-catalyzed condensation of 2 and pyrrole proceeded readily to give brominated porphyrin 3, which is sparingly soluble in MeCN. Washing the crude product with MeCN until the filtrate is colorless afforded analytically pure material in 48% yield. Eight phenyl rings were installed on the porphyrin via Suzuki-Miyaura coupling in 20:1 1,4-dioxane/water using a three-fold excess of PhB (OH)₂ (per Ar—Br bond), Cs₂CO₃ as a base, and 12.5 mol % (dppf) PdCl₂ (per Ar—Br bond). Silica gel chromatography afforded bulky porphyrin 4 as a deep purple solid in 86% yield. Single-crystal X-ray diffraction confirms the formation of the desired hydrophobic pocket (FIG. 16).

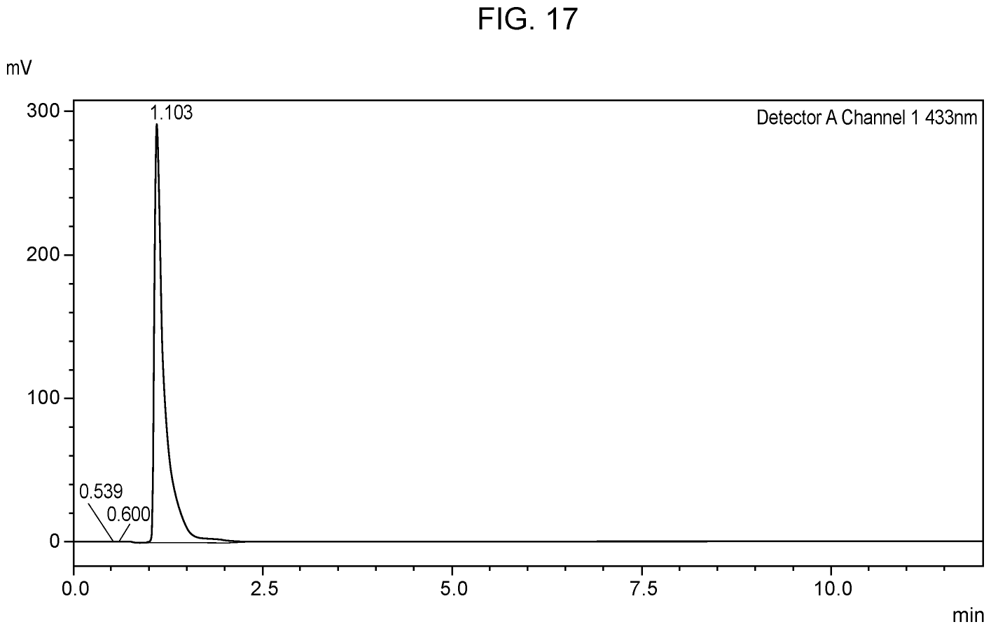

Iron was inserted into 4 by refluxing it with Fe(CO)₅ and a catalytic amount of Iz in toluene under N₂ for 4 h; an additional hour of reflux in open air followed by an alkaline aqueous work-up ensured oxidation to the Fe(III) state. ¹H NMR spectroscopy in CDCl₃ confirms that product 5 is paramagnetic, with broadened phenyl resonances appearing in the 15-20 ppm range and the B-pyrrole protons characteristically resonating at 82.12 ppm. Crystals of the reaction product grown from MeCN confirmed the proposed connectivity and were consistent with the presence of an apical hydroxide ligand (FIG. 6). Compound 5 was treated with Me₃SiOSO₂Cl in CCl₄ followed by hydrolysis with 1 M NaOH_{(aq)}. The intensely colored porphyrin complex was extracted from the reaction mixture with water and subsequently purified by reverse-phase chromatography. The final tetrasodium salt 6 was isolated as a very dark purple solid in 40% yield. The purity of the product was established using analytical HPLC (FIG. 17). ¹H NMR spectroscopy in DMSO-do confirmed that the compound is paramagnetic, with the B-pyrrole protons appearing at 80.41 ppm. In phosphate-buffered saline (PBS; pH 7.4), the Soret band appears at 431 nm. Weakly diffracting crystals confirmed the proposed connectivity, including the apical hydroxide ligand (FIG. 7). High-quality crystals that were grown from DMSO/CHCl₃ permitted atomic-resolution refinement that confirmed the proposed structure, albeit following axial ligand substitution (FIG. 18).

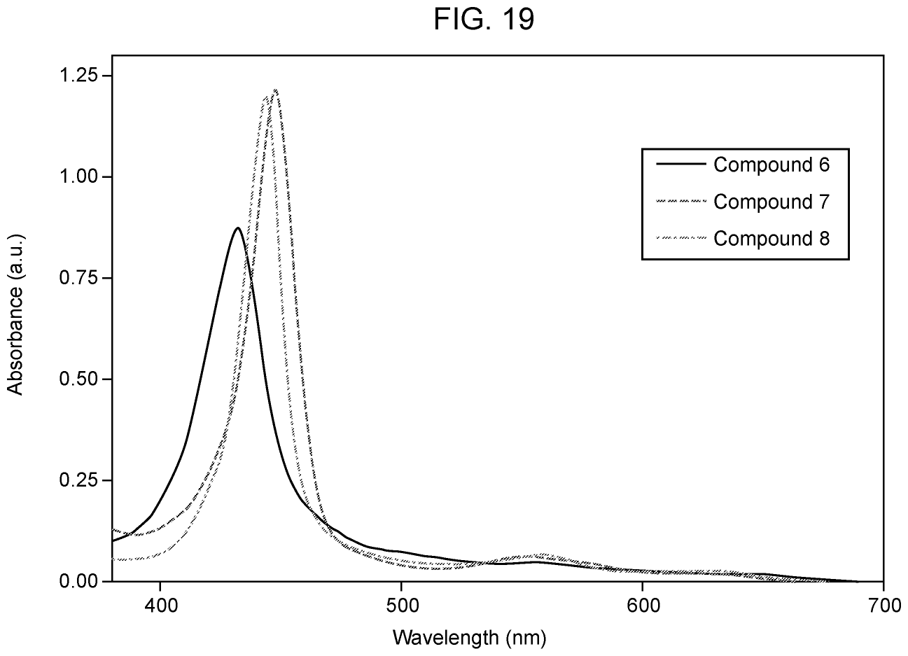

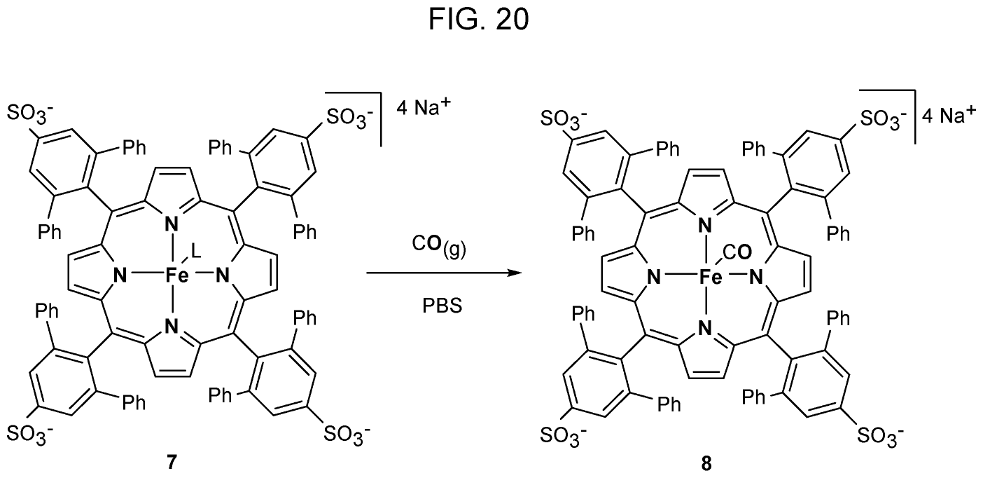

Compound 6 can be reduced in situ using Na₂S₂O₄ to afford the Fe(II) complex 7 (FIG. 15). Upon reduction, the Soret band shifts to 448 nm (FIG. 19). Under an inert atmosphere, solutions of 7 in PBS containing Na₂S₂O₄ are stable for days. When the solutions are opened to air, 7 reverts to 6 (t_{1/2}˜30 min) following aerial oxidation of the dithionite (FIG. S16). Addition of CO to solutions of 7 produces the CO adduct 8 (FIG. 20). Compound 8 is characterized by a Soret band at 444 nm in PBS (FIG. 19), consistent with formation of an Fe(II)-CO complex. In PBS solutions of 8 that have been opened to air, t_{1/2}˜120 min for reversion to 6 (FIG. 3). If the reduction of 6 under a CO atmosphere is performed in DMSO-d₆, ¹H NMR spectroscopy clearly shows 8 to be diamagnetic and Evans method measurements return a μ_{eff} of 0 μ_{B}. These results are consistent with the formation of a low-spin Fe(II) complex, which would be expected upon complexation of CO. Salt metathesis, effected by addition of excess (Ph₄P)Cl to an aqueous solution of 8, results in rapid precipitation of a red solid. IR spectroscopic analysis of this solid revealed a v_{CO} of 1970 cm⁻¹ (FIG. 5). The extent to which v_{CO} is decreased from that of free CO (2143 cm⁻¹) reflects the extent of Fe-to-CO backbonding, which in turn reflects how strongly the Fe center is binding the CO ligand. The higher value of v_{CO} for 8 as compared to COHb (1951 cm⁻¹) indicates that the small-molecule porphyrin complex binds CO more strongly than Hb.

This was tested by titrating COHb with 6 in PBS under reducing conditions (5.7 mM Na₂S₂O₄), which reduce it to 7 in situ. A dose-dependent and stoichiometric transfer of CO to form 8 and deoxyHb with tight isosbestic points was observed, indicating a clean transition from (COHb+7) to (deoxyHb+8) (FIG. 8). A mixture of deoxyHb and 7 with CO-saturated water was titrated. The first equivalent of CO converts 7 to 8 but leaves deoxyHb unchanged. Addition of a second equivalent of CO results in conversion of deoxyHb to COHb (FIG. 9). These experiments confirm that 7 can bind CO preferentially in the presence of Hb and sequester CO from COHb. All changes were completed between the time of mixing and spectral acquisition (20-30 s).

Finally, 7 was tested to determine whether it could sequester CO from poisoned bovine RBCs after confirming that 7 does not induce hemolysis (FIG. 10). Spectroscopic titrations were performed directly on suspensions of CO-treated RBCs in PBS containing 5.7 mM dithionite. The intensity of Soret band of COHb (max=420 nm) decreased from the concentration-predicted absorbance because of the inner filter effect introduced by concentration of the Hb into the RBCs. Accurate concentrations of COHb were obtained by measuring the absorbance of lysed aliquots of the suspension. Titration with 6, which is reduced in situ to 7, resulted in a dose-dependent decrease in the intensity of the COHb signal, and an increase of the signal for 8 (λₘₐₓ=444 nm) (FIG. 11). To confirm that CO had indeed been abstracted from Hb, CO was bubbled through the suspension of RBCs treated with 1 equiv of 6; the COHb signal was regenerated. These results represent the first demonstration of a small molecule acting as a CO-poisoning antidote. Preliminary insight was gained by monitoring absorbance at 420 nm of a quiescent suspension of CO-poisoned RBCs in a buffered dithionite solution following addition of 1 equiv of 6. The reaction was essentially complete within 3 min (FIG. 12).

Water soluble metal porphyrin complexes as described herein have demonstrated the viability of the synthetic strategy, an affinity for CO, an ability to remove CO from COHb, and the capacity to sequester CO from poisoned RBCs. This activity is performed without any damage to the cells.

### Example 2

### Synthesis and Functionalization of Meso-Substituted Aryl Bis-Pocket Pophyrins Accessed Via Suzuki-Miyaura Cross Coupling

Synthetic routes to porphyrin derivatives have been invaluable in increasing understanding of the biology of these molecules and in providing access to porphyrin compounds that have tremendous potential as catalysts and functional materials. Porphyrins with bulky substituents that leave hydrophobic pockets above and below the porphyrin plane are called “bis-pocket” porphyrins and have been used to prepare small-molecule models of metalloprotein active sites, isolate reactive intermediates, and form size-selective oxidation catalysts. Bis-pocket porphyrin syntheses that rely on the condensation of bulky terphenylaldehyde derivatives with pyrrole can in some instances suffer from low yields arising from the steric encumbrance of the starting aldehydes.

Example 2 presents the synthesis, metalation, and functionalization of bis-pocket porphyrins using the Suzuki-Miyaura cross coupling reaction. Steric limitations to accessing bis-pocket porphyrins were overcome by using this Pd-catalyzed C-C bond forming strategy to introduce steric bulk after macrocyclization: 2,6-dibromo-4-trimethylsilybenzaldehyde was condensed with pyrrole and a variety of boronic acids were coupled to the resulting porphyrin in up to 95% yield. Example 2 demonstrates that the porphyrins can be metalated with a variety of metals and sulfonated to create water-soluble bis-pocket porphyrins.

Example 2 describes a method which allows a variety of different groups varying in sterics, electronics, and functional group presentation to be coupled to the porphyrin framework.

The TMS groups on the porphyrin derivatives provide excellent organic solubility, even when large aromatic groups are installed. An optimization of the sulfonation reaction is also provided.

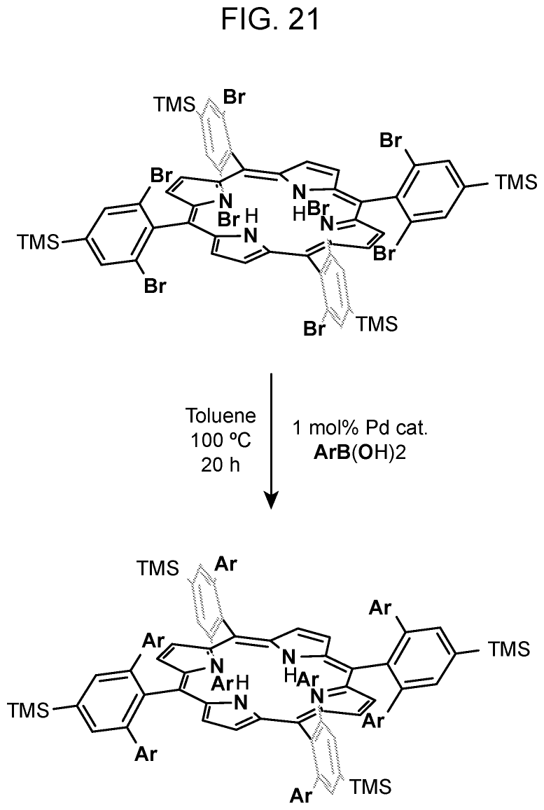

In this reaction, the TMS groups are exchanged for SO3-groups that confer water solubility on these bulky porphyrins. Finally, although the bulky substituents can inhibit the insertion of some metals into these porphyrins, refluxing a metal halide, 2,6-lutidine, and the free-base ligand in 1,2,4-trichlorobenzene (1,2,4-TCB) permits facile and rapid metalation. FIG. 21 depicts a general scheme for bis-pocket porphyrin synthesis as described in Example 2.

### Experimental Section

General methods. All reactions were performed under N₂ unless otherwise specified. Glassware was oven dried prior to use. All solvents and reagents are commercially available and used as received unless otherwise stated. Compound 2-1 was prepared as described above, such as in Example 1. Suzuki-Miyaura reactions were performed in Chemglass 20-mL reaction vials fitted with pressure relief caps and heated on a hot plate fitted with a Chemglass 4-place pie wedge for 20-mL scintillation vials. For the purification of 2-3a and 2-3b, an Isolera Prime Biotage fitted with a Sfär C18 column was employed. A solution of 1% triethylammonium bicarbonate in water was generated by dissolving 40 mL of triethylamine in 4 L of ultra-pure (UP) water (>18 M (2 cm) followed by the addition of 150 g of dry ice. Organic solutions were concentrated under reduced pressure on a Buchi Rotavapor R-100. CDC13 and DMSO-do were purchased from Cambridge Isotope Laboratories and used as received. ¹H, ¹³C{¹H}, and ¹⁹F{H} NMR spectra were recorded on a Bruker Avance III HD 500 NMR spectrometer equipped with a multinuclear Smart Probe. Signals in the ¹H and ¹³C NMR spectra are reported in ppm as chemical shifts from tetramethylsilane; ¹⁹F NMR signals are reported in ppm as chemical shifts from CFCl₃. NMR signals were referenced using the CHCl₃ (¹H, 7.26 ppm), DMSO-d₅ (¹H, 2.50 ppm), or CDCl₃ (¹³C, 77.0 ppm) solvent signals. The following abbreviations were used to explain the multiplicities: s=singlet, d=doublet, t=triplet, q=quartet, m=multiplet, sext =sextet. Solution phase magnetic moments were measured using a modified Evans method. {Schubert, 1992 #1086} UV-visible absorption spectra were measured on a VWR UV-6300PC dual-beam spectrophotometer. MALDI mass spectra were acquired using timsControl v 1.1.19 on a timsTOF fleX mass spectrometer (Bruker Scientific, Billerica, MA) over the mass range 1000-2500 Da. In positive reflectron mode, laser power was set to 20%, and laser application was set to MS Dried Droplet. In negative reflectron mode, laser power was set to 30%, and laser application was set to MS Dried Droplet. Compounds were dissolved in DCM or MeOH and 1 μL was mixed with 1 μL of matrix (50:50 α-cyano-4-hydroxycinnamic acid: 2,5-dihydroxybenzoic acid in a solution of 70:30 ACN: H₂O with 0.1% trifluoroacetic acid). Samples were spotted on a stainless steel MSP 96 spot target plate and allowed to air dry. For each compound, 1000 laser shots at 2000 Hz were delivered in a random walk across the spot. Data were subsequently analyzed in DataAnalysis v 5.3 (Bruker Scientific, Billerica, MA).

The description of the synthesis of the compounds in Example 2 make reference to the Scheme 2-1 shown below:

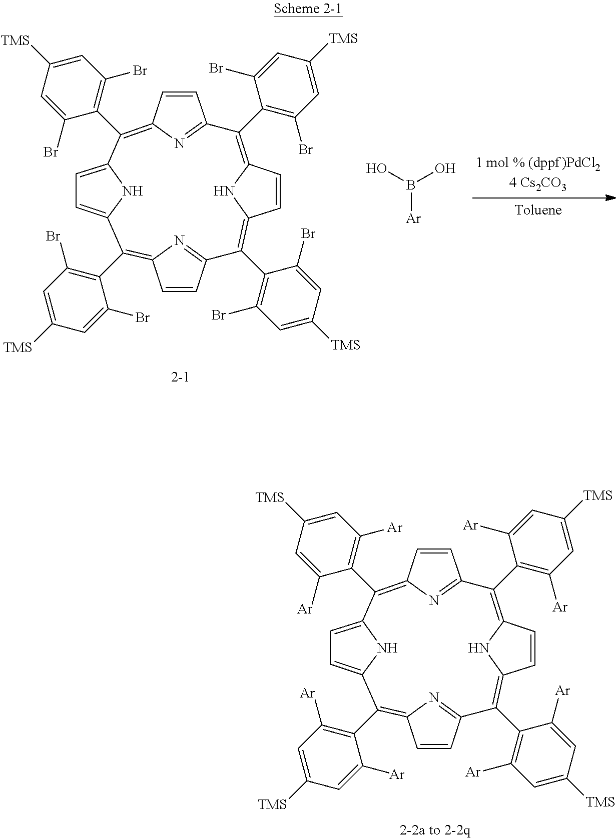

General method for Suzuki-Miyaura coupling. Compound 2-1 (100 mg, 0.0656 mmol), 1 mol % per carbon-bromine bond of palladium catalyst (0.0052 mmol), 3 equiv of boronic acid (1.574 mmol), and 4 equiv of cesium carbonate (2.09 mmol) were dissolved in a mixture of toluene (5 mL) and DI water (0.2 mL) in a 20-mL reaction vial fitted with a pressure relief cap. The mixture was sparged with N₂ for 5 min, then sealed and brought to 100° C. and stirred for 20 h. The crude reaction mixture was dry loaded onto silica and purified by normal phase flash chromatography. The eluted product was concentrated under reduced pressure, then redissolved in a minimal amount of chloroform and recrystallized overnight via layering with MeCN. The resulting purple crystals were isolated via vacuum filtration.

Synthesis of Compound 2-2a 5,10,15,20-tetrakis(2,6-diphenyl-4-(trimethylsilyl) phenyl) porphyrin. Synthesized using the general method for Suzuki-Miyaura couplings. Column chromatography run as a ramp to 50% CHCl₃ in hexanes. Compound 2-2a was isolated as purple crystals (88 mg, 89%). Characterization was consistent with previously reported data. ^{21 1}H NMR (500 MHZ, CDCl₃) δ8.38 (s, 8H), 7.77 (s, 8H), 6.55 (d, J=7.9 Hz, 16H), 6.40 (t, J=7.2 Hz, 8H), 6.22 (t, J=7.5 Hz, 16H), 0.50 (s, 36H), −3.46 (s, 2H). ¹³C{H} NMR (126 MHz, CDCl₃) δ144.78, 142.40, 140.77, 139.34, 133.63, 129.42, 126.66, 125.25, 116.13, −0.63; HRMS (MALDI) m/z: [M+H]⁺ Calcd for C₁₀₄H₉₅N₄Si₄⁺ 1512.6662; Found 1512.6669; UV/Vis (CHCl₃) λ_{abs} (log ε): 420 (sh), 439 (5.67), 533 (4.32), 570 (4.01), 611 (3.42), 669 (3.48).

Synthesis of Compound 2-2b 5,10,15,20-tetrakis(2,6-di(3,5-difluoro)phenyl-4-(trimethylsilyl) phenyl) porphyrin. Synthesized using the general method for Suzuki-Miyaura couplings. Column chromatography run as a ramp to 40% CHCl₃ in hexanes. Compound 2-2b was isolated as purple crystals (59 mg, 50%). X-ray quality crystals were grown by layering MeCN over a solution of the product in CHCl₃. ¹H NMR (500 MHZ, CDCl₃) δ8.42 (s, 8H), 7.80 (s, 8H), 6.06 (d, J=4.4 Hz, 16H), 5.87-5.79 (m, 8H), 0.54 (s, 36H), −3.16 (s, 2H). ¹³C{H} NMR (126 MHz, CDCl₃) δ162.61, 162.51, 160.64, 160.53, 144.60, 143.15, 142.26, 138.67, 134.03, 115.67, 112.31, 112.13, 101.78, 101.59, 101.39, −0.72. ¹⁹F{H} NMR (470 MHz, CDCl₃) 8-111.49. HRMS (MALDI) m/z: [M+H]⁻ Calcd for C₁₀₄H₇₉F₁₆N₄Si⁺ 1800.5155; Found 1800.5142; UV/Vis (CHCl₃) λ_{abs} (log ε): 417 (sh), 435 (5.66), 430 (4.32), 565 (3.98), 608 (3.79), 667 (3.47).

Synthesis of Compound 2-2c 5,10,15,20-tetrakis(2,6-di(3,5-dichloro)phenyl-4-(trimethylsilyl) phenyl) porphyrin. Synthesized using the general method for Suzuki-Miyaura couplings, but with Pd (PPh₃)₄ as the catalyst. Column chromatography run as a ramp to 10% toluene in pentane. Compound 2-2c was isolated as purple crystals (74 mg, 70%). X-ray quality crystals were grown by layering MeCN over a solution of the product in CHCl₃. ¹H NMR (500 MHz, CDCl₃) δ8.47 (s, 8H), 7.76 (s, 8H), 6.45 (d, J=1.8 Hz, 16H), 6.39 (t, J=1.8 Hz, 8H), 0.54 (s, 36H), −2.93 (s, 2H). ¹³C{H} NMR (126 MHz, CDCl₃) δ144.31, 142.66, 142.38, 138.18, 134.91, 133.54, 127.51, 126.33, 115.56, −0.69. HRMS (MALDI) m/z: [M+H]⁺ Calcd for C₁₀₄H₇₉Cl₁₆N+Si₄⁺ 2064.0309; Found 2064.0280; UV/Vis (CHCl₃) λ_{abs} (log ε): 419 (sh), 438 (5.58), 490 (4.06), 530 (4.26), 567 (3.91), 608 (3.72), 668 (3.41)

Synthesis of Compound 2-2d 5,10,15,20-tetrakis(2,6-di(3,5-dimethyl)phenyl-4-(trimethylsilyl) phenyl) porphyrin. Synthesized using the general method for Suzuki-Miyaura couplings. Column chromatography was performed as a ramp to 30% chloroform in hexanes. Compound 2-2d was isolated as purple crystals (108 mg, 95%). X-ray quality crystals were grown by layering MeCN over the product in 1,2,4-TCB to give purple plates. ¹H NMR (500 MHz, CDCl₃) δ8.51 (s, 8H), 7.71 (s, 8H), 6.20 (s, 16H), 5.92 (s, 8H), 1.15 (s, 48H), 0.49 (s, 36H), −3.04 (s, 2H). ¹³C{H} NMR (126 MHz, CDCl₃) δ145.12, 142.52, 140.28, 138.66, 135.74, 134.63, 127.48, 127.30, 116.69, 20.73, −0.59. HRMS (MALDI) m/z: [M+H]⁺ Calcd for C₁₂₀H₁₂₇N₄Si₄⁺ 1736.9166; Found 1736.9185; UV/Vis (CHCl₃) λ_{abs} (log ε): 418 (sh), 437 (5.66), 532 (4.28), 567 (3.99), 609 (3.74),669 (3.44).

Synthesis of Compound 2-2e 5,10,15,20-tetrakis(2,6-di(4-methyl)phenyl-4-(trimethylsilyl) phenyl) porphyrin. Synthesized using the general method for Suzuki-Miyaura couplings. Column chromatography was performed as a ramp to 40% chloroform in hexanes. Compound 2-2e was isolated as purple crystals (76 mg, 71%). X-ray quality crystals were grown by layering MeCN over the product in toluene to give purple plates. ¹H NMR (500 MHZ, CDCl₃) δ8.43 (s, 8H), 7.72 (s, 8H), 6.51 (d, J=8.1 Hz, 16H), 6.05 (d, J=8.1 Hz, 16H), 1.70 (s, 24H), 0.50 (s, 36H), −3.31 (s, 2H). ¹³C{H} NMR (126 MHz, CDCl₃) δ144.68, 140.50, 139.76, 139.33, 134.28, 133.69, 129.30, 127.45, 116.33, 20.78, −0.63. HRMS (MALDI) m/z: [M+H]⁺ Calcd for C₁₁₂H₁₁₁N₄Si₄⁺ 1624.7914; Found 1624.7885; UV/Vis (CHCl₃) λ_{abs} (log ε): 420 (sh), 439 (5.64), 533 (4.28), 569 (3.95), 610 (3.75), 671 (3.39).

Synthesis of Compound 2-2f 5,10,15,20-tetrakis(2,6-di(4-n-propyl)phenyl-4-(trimethylsilyl) phenyl) porphyrin. Synthesized using the general method for Suzuki-Miyaura couplings. Column chromatography was performed as a ramp to 20% diethyl ether in hexanes. Compound 2-2f was not recrystallized and was isolated as a purple solid (85 mg, 70%). X-ray quality crystals were grown by layering a one-to-one mixture of EtOH and MeCN over the product in CHCl₃ to give purple needles. ¹H NMR (500 MHZ, CDCl₃) δ8.38 (s, 8H), 7.74 (s, 8H), 6.46 (d, J=7.5 Hz, 16H), 6.03 (d, J=8.2 Hz, 16H), 1.94-1.82 (m, 16H), 1.11 (sext, J=7.4 Hz, 16H), 0.53-0.45 (m, 60H), −3.28 (s, 2H). ¹³C{H} NMR (126 MHZ, CDCl₃) δ139.96, 139.11, 133.80, 129.33, 126.73, 37.72, 24.43, 13.78, −0.59. HRMS (MALDI) m/z: [M+H]⁺ Calcd for C₁₂₈H₁₄₃N₄Si₄⁺ 1849.0417; Found 1849.0427; UV/Vis (CHCl₃) λ_{abs} (log ε): 420 (sh), 440 (5.63), 534 (4.27), 570 (3.95), 611 (3.74), 671 (3.37).

Synthesis of Compound 2-2g 5,10,15,20-tetrakis(2,6-di(4-tert-butyl)phenyl-4-(trimethylsilyl) phenyl) porphyrin. Synthesized using the general method for Suzuki-Miyaura couplings but with the reaction time extended to 48 h. Column chromatography was performed as a ramp to 5% chloroform in hexanes. Compound 2-2g was not recrystallized and was isolated as a purple solid (57 mg, 44%). ¹H NMR (500 MHZ, CDCl₃) δ8.38 (s, 8H), 7.82 (s, 8H), 6.42-6.30 (m, 32H), 0.77 (s, 72H), 0.52 (s, 36H), −2.97 (s, 2H). ¹³C{H} NMR (126 MHz, CDCl₃) δ147.67, 145.13, 140.45, 140.00, 138.73, 135.25, 129.47, 123.97, 116.12, 33.90, 31.30, −0.58. HRMS (MALDI) m/z: [M+H]⁺ Calcd for C₁₃₆H₁₅₉N₄Si₄⁺ 1961.1669; Found 1961.1690; UV/Vis (CHCl₃) λ_{abs} (log ε): 422 (sh), 443 (5.68), 537 (4.30), 573 (4.07), 612 (3.79), 672 (3.43).

Synthesis of Compound 2-2h 5,10,15,20-tetrakis(2,6-di(4-fluoro)phenyl-4-(trimethylsilyl) phenyl) porphyrin. Synthesized using the general method for Suzuki-Miyaura couplings. Column chromatography was performed as a ramp to 30% chloroform in hexanes. Compound 2-2h was isolated as purple crystals (67 mg, 62%). X-ray quality crystals were grown by layering MeCN over the product in toluene at −20° C. to give purple plates. ¹H NMR (500 MHz, CDCl₃) δ8.38 (s, 8H), 7.75 (s, 8H), 6.52 (dd, J=8.7, 5.4 Hz, 16H), 5.95 (t, J=8.7 Hz, 16H), 0.52 (s, 36H), −3.35 (s, 2H). 13C{H} NMR (126 MHz, CDCl₃) δ161.64, 159.68, 143.93, 141.40, 139.23, 138.19, 138.17, 133.80, 130.91, 130.84, 116.25, 113.71, 113.54, −0.66. ¹⁹F{H} NMR (470 MHz, CDCl₃) δ−116.48. HRMS (MALDI) m/z: [M+H]⁺ Calcd for C₁₀₄H₈₇F₈N₄Si₄⁺ 1656.5909; Found 1656.5879; UV/Vis (CHCl₃) λ_{abs} (log ε): 419 (sh), 439 (5.66), 533 (4.30), 569, (3.99), 611 (3.76), 670 (3.45).

Synthesis of Compound 2-2i 5,10,15,20-tetrakis(2,6-di(4-trifluoromethyl)phenyl-4-(trimethylsilyl) phenyl) porphyrin. Synthesized using the general method for Suzuki-Miyaura couplings. Column chromatography was performed as a ramp to 10% chloroform in hexanes. Compound 2-2i was not recrystallized and was isolated as a purple solid (37 mg, 27%). X-ray quality purple needles were grown by layering MeCN over a solution of the product in CHCl₃. ¹H NMR (500 MHZ, CDCl₃) δ8.40 (s, 8H), 7.80 (s, 8H), 6.59 (d, J=8.1 Hz, 16H), 6.47 (d, J=8.2 Hz, 16H), 0.52 (s, 36H), −3.33 (s, 2H). 19F{H} NMR (470 MHz, CDCl₃) δ−63.27. ¹³C{H} NMR (126 MHz, CDCl₃) δ145.46, 143.83, 142.20, 138.54, 134.46, 129.47, 128.24, 127.98, 126.67, 126.49, 124.51, 123.64, 122.34, 120.18, 115.88, −0.73. HRMS (MALDI) m/z: [M+H]⁺ Calcd for C₁₁₂H₈₇F₂₄N₄Si₄⁺ 2056.5652; Found 2056.5643; UV/Vis (CHCl₃) λ_{abs} (log ε): 418 (sh), 437 (5.61), 495 (3.65), 531 (4.26), 567 (3.96), 608 (3.79), 668 (3.48), 700 (2.98).

Synthesis of Compound 2-2j 5,10,15,20-tetrakis(2,6-di(4-nitro)phenyl-4-(trimethylsilyl) phenyl) porphyrin. Synthesized using the general method for Suzuki-Miyaura couplings, but with Pd (PPh₃)₄ as the catalyst, 4 equiv of boronic acid, and 48 h reaction time. Column chromatography was performed as a slow ramp to 100% chloroform in hexanes. Compound 2-2j was not recrystallized and isolated as a purple solid (5.7 mg, 5%). ¹H NMR (500 MHz, CDCl₃) δ8.38 (s, 8H), 7.84 (s, 8H), 7.11 (d, J=8.9 Hz, 16H), 6.58 (d, J=8.9 Hz, 16H), 0.54 (s, 36H), −3.33 (s, 2H). ¹³C{H} NMR (126 MHZ, CDCl₃) δ148.23, 145.90, 143.29, 137.87, 135.01, 130.06, 122.11, 115.86, −0.76. HRMS (MALDI) m/z: [M+H]⁺ Calcd for C₁₀₄H₈₇N₁₂O₁₆Si₄⁺ 1872.5468; Found 1872.5437; UV/Vis (CHCl₃) λ_{abs} (log ε): 423 (sh), 445 (5.42), 536 (4.14), 573 (3.85), 612 (3.60), 671 (3.21).

Synthesis of Compound 2-2k 5,10,15,20-tetrakis(2,6-di(4-methoxy)phenyl-4-(trimethylsilyl) phenyl) porphyrin. Synthesized using the general method for Suzuki-Miyaura couplings, but with diglyme as the solvent. After reaction completion, the crude mixture was diluted with hexanes (50 mL) and loaded onto a silica column. The loaded column was washed with hexanes, then 100 mL of 1:1 chloroform/hexanes and then 100 mL of 100% chloroform. The product was then eluted by ramping to 15% methanol in chloroform. Compound 2-2k was not recrystallized and was isolated as a purple solid (92 mg, 80%). X-ray quality crystals were grown by layering MeCN over the product in CHCl₃ to give purple needles. ¹H NMR (500 MHZ, CDCl₃) δ8.40 (s, 8H), 7.72 (s, 8H), 6.46 (d, J=8.8 Hz, 16H), 5.78 (d, J=8.9 Hz, 16H), 3.17 (s, 24H), 0.50 (s, 36H), −3.27 (s, 2H). ¹³C{H} NMR (126 MHz, CDCl₃) δ157.17, 144.53, 140.52, 139.62, 135.02, 133.49, 130.43, 116.52, 112.21, 54.56, −0.60. HRMS (MALDI) m/z: [M+H]⁺ Calcd for C₁₁₂H₁₁₁N₄O₈Si₄⁺1752.7507; Found 1752.7490; UV/Vis (CHCl₃) λ_{abs} (log ε): 422 (sh), 442 (5.57), 497 (3.76), 535 (4.23), 572 (3.97), 612 (3.74), 671 (3.45).

Synthesis of Compound 2-21 5,10,15,20-tetrakis(2,6-di(2-napthyl)phenyl-4-(trimethylsilyl) phenyl) porphyrin. Synthesized using the general method for Suzuki-Miyaura couplings. Column chromatography was performed as a ramp to 50% chloroform in hexanes. Compound 2-21 was isolated as purple crystals (85.3 mg, 68%). X-ray quality crystals were grown by layering MeCN over a solution of the product in CHCl₃. ¹H NMR (500 MHZ, CDCl₃) δ8.47 (s, 8H), 7.79 (s, 8H), 7.32 (s, 8H), 7.13-7.06 (m, 16H), 6.94 (t, J=7.3 Hz, 8H), 6.81 (d, J=8.3 Hz, 8H), 5.94 (d, J=8.3 Hz, 8H), 5.26 (d, J=8.6 Hz, 8H), 0.51 (s, 36H), −3.56 (s, 2H). ¹³C{H} NMR (126 MHz, CDCl₃) 8 144.81, 140.90, 140.10, 139.58, 134.08, 132.43, 131.03, 127.77, 127.52, 127.23, 126.89, 125.34, 125.08, 115.99, −0.61. HRMS (MALDI) m/z: [M+H]⁺ Calcd for C₁₃₆H₁₁₁N₄Si₄⁺ 1912.7913; Found 1912.7938; UV/Vis (CHCl₃) λ_{abs} (log ε): 423 (sh), 444 (5.58), 536 (4.24), 571 (3.93), 611 (3.72), 671 (3.38).

Synthesis of Compound 2-2m 5,10,15,20-tetrakis(2,6-dicyclopropyl-4-(trimethylsilyl) phenyl) porphyrin. Synthesized using the general method for Suzuki-Miyaura couplings. Column chromatography was performed as a ramp to 50% chloroform in hexanes. Compound 2-2m was not recrystallized and was isolated as a purple solid (26 mg, 32%). X-ray quality crystals were grown by layering MeCN over the product in CHCl₃ to give purple needles. ¹H NMR (500 MHZ, CDCl₃) δ8.66 (s, 8H), 7.22 (s, 8H), 1.15-1.09 (m, 8H), 0.67-0.61 (m, 16H), 0.46 (s, 36H), 0.05-0.03 (m, 16H), −2.29 (s, 2H). ¹³C{H} NMR (126 MHZ, CDCl₃) δ143.41, 143.25, 140.48, 125.21, 117.28, 15.75, 8.72, −0.60. HRMS (MALDI) m/z: [M+H]⁺ Calcd for C₈₀H₉₅N₄Si₄⁺ 1223.6628; Found 1223.6599; UV/Vis (CHCl₃) λ_{abs} (log ε): 404 (sh), 422 (5.55), 516 (4.16), 550 (3.62), 591 (3.63), 646 (3.26).

Synthesis of Compound 2-2n 5,10,15,20-tetrakis(2,6-di(4-vinyl)phenyl-4-(trimethylsilyl) phenyl) porphyrin. Synthesized using the general method for Suzuki-Miyaura couplings. Column chromatography was performed as a ramp to 30% chloroform in hexanes. Compound 2-2n was not recrystallized and was isolated as a purple solid (37 mg, 33%). ¹H NMR (500 MHZ, CDCl₃) δ8.42 (s, 8H), 7.76 (s, 8H), 6.49 (d, J=8.2 Hz, 16H), 6.27 (d, J=8.3 Hz, 16H), 6.03 (dd, J=17.6, 10.9 Hz, 8H), 5.14 (d, J=17.5 Hz, 8H), 4.77 (d, J=11.3 Hz, 8H), 0.51 (s, 36H), −3.27 (s, 2H). ¹³C{H} NMR (126 MHz, CDCl₃) δ144.70, 142.07, 140.86, 139.22, 136.33, 134.44, 133.75, 129.56, 124.79, 116.21, 113.07,−0.65. HRMS (MALDI) m/z: [M+H]⁺ Calcd for C₁₂₀H₁₁₁N₄Si₄⁺ 1720.7914; Found 1720.7893; UV/Vis (CHCl₃) λ_{abs} (log ε): 422 (sh), 443 (5.38), 536 (4.05), 571 (3.76), 612 (3.55), 672 (3.25).

Synthesis of Compound 2-20 5,10,15,20-tetrakis(2,6-di(4-trimethylsilyl)phenyl-4-(trimethylsilyl) phenyl) porphyrin. Synthesized using the general method for Suzuki-Miyaura couplings, but with 4 equiv of boronic acid. Column chromatography was performed as a ramp to 5% toluene in hexanes. Compound 2-20 was not recrystallized and was isolated as a purple solid (64 mg, 47%). X-ray quality crystals were grown by layering a one-to-one mixture of EtOH and MeCN over the product in CHCl₃ to give purple needles. ¹H NMR (500 MHZ, CDCl₃) δ8.38 (s, 8H), 7.79 (s, 8H), 6.52 (d, J=7.6 Hz, 16H), 6.42 (d, J=7.4 Hz, 16H), 0.50 (s, 36H), −0.26 (s, 72H), −3.05 (s, 2H). ¹³C{H} NMR (126 MHz, CDCl₃) δ145.41, 143.53, 140.62, 138.52, 136.46, 135.70, 132.33, 129.29, 115.68, −0.62, −0.88. HRMS (MALDI) m/z: [M+H]⁺ Calcd for C₁₂₈H₁₅₉N₄Si₁₂⁺ 2088.9824; Found 2088.9763; UV/Vis (CHCl₃) λ_{abs} (log ε): 424 (sh), 444 (5.63), 538 (4.31), 574 (4.07), 612 (3.85), 671 (3.52).

Synthesis of Compound 2-2p 5,10,15,20-tetrakis(2,6-di(4-ethoxycarbonyl)phenyl-4-(trimethylsilyl) phenyl) porphyrin. Synthesized using the general method for Suzuki-Miyaura couplings, but with diglyme as the solvent and 4 equiv of boronic acid. After reaction completion, the crude mixture was diluted with hexanes (50 mL) and loaded onto a silica column. The loaded column was washed with hexanes, then 100 mL of 1:1 chloroform/hexanes, and then 100 mL of 100% chloroform. The product was then eluted by ramping to 15% methanol in chloroform. Compound 2-2p was not recrystallized and was isolated as a purple solid (55 mg, 40%). X-ray quality crystals were grown by layering MeCN over the product in toluene at −20° C. to give purple plates. ¹H NMR (500 MHZ, CDCl₃) δ8.30 (s, 8H), 7.80 (s, 8H), 6.95 (d, J=8.0 Hz, 16H), 6.47 (d, J=7.9 Hz, 16H), 3.92 (q, J=7.0 Hz, 16H), 0.96 (t, J=7.0 Hz, 24H), 0.53 (s, 36H), −3.31 (s, 2H). ¹³C{H} NMR (126 MHz, CDCl₃) δ165.73, 146.84, 144.53, 141.56, 138.62, 134.75, 129.58, 128.14, 127.78, 115.60, 60.49, 14.08, −0.69. HRMS (MALDI) m/z: [M+H]⁺ Calcd for C₁₂₈H₁₂₇N₄O₁₆Si₄⁺2088.8353; Found 2088.8318; UV/Vis (CHCl₃) λ_{abs} (log ε): 424 (sh), 444 (5.64), 537 (4.31), 574 (4.09), 613 (3.83), 672 (3.61).

Synthesis of Compound 2-2q 5,10,15,20-tetrakis(2,6-di(N-methylpyrrazolyl)phenyl-4-(trimethylsilyl)phenyl)porphyrin. Synthesized using the general method for Suzuki-Miyaura couplings, but with diglyme as the solvent and 4 equiv of boronic acid. After reaction completion, the crude mixture was diluted with hexanes (50 mL) and loaded onto a silica column. The loaded column was washed with hexanes, then 100 mL of 1:1 chloroform/hexanes, and then 100 mL of 100% chloroform. The product was then eluted by ramping to 15% methanol in chloroform. Compound 2-2q was not recrystallized and was isolated as a purple solid (81 mg, 80%). X-ray quality crystals were grown by layering diethyl ether over the product in CHCl₃ to give purple plates. ¹H NMR (500 MHZ, CDCl₃) δ8.51 (s, 8H), 7.79 (s, 8H), 6.29 (s, 8H), 5.73 (s, 8H), 2.88 (s, 24H), 0.51 (s, 36H), −2.64 (s, 2H). ¹³C{H} NMR (126 MHZ, CDCl₃) δ141.35, 137.99, 137.62, 135.63, 131.70, 128.57, 123.02, 118.04, 38.17, −0.70. HRMS (MALDI) m/z: [M+H]⁺ Calcd for C₈₈H₉₅N₂₀Si₄⁺ 1543.7120; Found 1543.7139; UV/Vis (CHCl₃) λ_{abs} (log ε): 412 (sh), 431 (5.57), 524 (4.27), 559 (3.71), 598 (3.78), 656 (3.4).

### Sulfonations

Synthesis of Compound 2-3a sodium 5,10,15,20-tetrakis(2,6-diphenyl-4-(sulfonato)phenyl)porphyrin. Compound 2-2a (50 mg, 0.0331 mmol) and 4.8 equiv of trimethylsilyl chlorosulfonate (24 μL, 0.1589 mmol) were dissolved in carbon tetrachloride (5mL) in a 20-mL reaction vial fitted with a pressure release cap. The reaction was sealed and incubated at 75° C. for 2 h. The reaction was removed from heat and quenched with 5 mL of 1 M NaOH (aq) and stirred vigorously for 30 min. The crude mixture was concentrated under reduced pressure and purified by reverse phase flash column chromatography using a ramp to 95% acetonitrile in water with 1% triethylammonium bicarbonate. The eluted product was diluted with 20 mL of brine and dialyzed overnight against DI water through a 3.5 kDa MWCO membrane. The solution was concentrated under reduced pressure and Compound 2-3a was isolated as a purple solid (32 mg, 60%). ¹H NMR (500 MHZ, DMSO-d₆) δ8.41 (s, 8H), 7.87 (s, 8H), 6.50 (d, J=7.7 Hz, 16H), 6.42 (t, J=7.2 Hz, 8H), 6.28 (t, J=7.5 Hz, 16H), −3.69 (s, 2H). ¹³C{H} NMR (126 MHZ, DMSO-d₆) δ148.22, 144.39, 141.14, 137.72, 128.52, 126.75, 125.68, 125.64, 115.48; HRMS (MALDI) m/z: [M−4Na+3H]⁺ Calcd for C₉₂H₆₁N₄O₁₂S₄⁺ 1541.3174; Found 1541.3159; UV/Vis (CHCl₃) λ_{abs} (log ε): 416 (sh), 435 (5.29), 529 (3.94), 565 (3.48), 606(3.40), 665 (2.96)

Synthesis of Compound 2-3b sodium 5,10,15,20-tetrakis(2,6-di(3,5-difluoro)phenyl-4-(sulfonato)phenyl)porphyrin. Compound 2-2b (50 mg, 0.0278 mmol) and 12 equiv of trimethylsilyl chlorosulfonate (51 μL, 0.334 mmol) were dissolved in carbon tetrachloride (5mL) in a 20-mL reaction vial fitted with a pressure release cap. The reaction was sealed and incubated at 75° C. for 1 h. The reaction was taken off heat and quenched with 5 mL of IM NaOH (aq) and stirred vigorously for 30 min. The crude mixture was concentrated under reduced pressure and purified by reverse phase flash column chromatography using a ramp to 95% acetonitrile in water with 1% triethylammonium bicarbonate. The eluted product was diluted with 20 mL of brine and dialyzed overnight against DI water through a 3.5 kDa MWCO membrane. The solution was concentrated under reduced pressure and Compound 2-3b was isolated as a purple solid (45 mg, 84%). X-ray quality crystals were grown by vapor diffusion of diethyl ether into a solution of the product 1:1 methanol/diglyme to give purple plates. HRMS (MALDI) m/z: [M−4Na+3H]⁺ Calcd for C₉₂H₄₅F₁₆N₄O₁₂S₄⁺ 1829.1667; Found 1829.1639; ¹H NMR (500 MHZ, DMSO-d₆) δ8.58 (s, 8H), 7.96 (s, 8H), 6.29-6.01 (m, 24H), −3.44 (s, 2H). ¹⁹F{H} NMR (470 MHz, DMSO-d₆) δ−110.91. ¹³C{H} NMR (126 MHZ, DMSO-d₆) δ161.85, 161.76, 159.89, 159.78, 148.85, 143.87, 142.48, 137.26, 126.30, 114.77, 111.73, 111.54, 101.89, 101.67, 101.48; UV/Vis (CHCl₃) λ_{abs} (log ε): 414 (sh), 432 (5.29), 527 (3.94), 568 (3.48), 607 (3.40), 665 (3.36)

Synthesis of Compound 2-4a 5,10,15,20-tetrakis(2,6-diphenyl-4-(trimethylsilyl) phenyl) porphyrinatoaquazine(II). Compound 2-2a (50 mg, 0.033 mmol), zinc(II) acetate dihydrate (700 mg, 3.20 mmol), pyridine (0.1 mL), DMF (5 mL), and a stir bar were added to a 15 mL round bottom flask outfitted with a reflux condenser under a stream of nitrogen. The reaction was heated to reflux and allowed to stir overnight (16 h). UV-vis spectroscopy was used to confirm product formation. The reaction was diluted with water and the resulting precipitate was collected by vacuum filtration. The solid was purified by column chromatography (silica gel, chloroform: hexanes 1:1). The product fractions were concentrated under reduced pressure to give the product as a blue purple solid (40 mg, 76%). X-ray quality crystals were grown by layering MeCN over the product dissolved in CHCl₃; ¹H NMR (500MHz, CDCl₃) δ8.48 (s, 8H), 7.76 (s, 8H), 6.62 (d, J=7.1 Hz, 16H), 6.37 (t, J=7.1 Hz, 8H), 6.20 (t, J=7.2 Hz, 16H), 0.51 (s, 36H), −1.29 (s, 2H). ¹³C{H} NMR (126 MHZ, CDCl₃) δ149.78, 144.63, 142.88, 140.32, 133.36, 131.48, 129.45, 126.44, 125.06, 116.65, −0.60; HRMS (MALDI) m/z: [M+H−OH]⁺ Calcd for C₁₀₄H₉₃N₄Si₄⁺ 1574.5796; Found 1574.5760; UV/Vis (CHCl₃) λ_{abs} (log ε): 422 (sh), 444 (5.58), 572 (4.22), 613 (3.50).

Synthesis of Compound 2-4b 5,10,15,20-tetrakis(2,6-diphenyl-4-(trimethylsilyl) phenyl) porphyrinatocopper(II). Compound 2-2a (50 mg, 0.033 mmol), copper(II) chloride dihydrate (563 mg, 3.30 mmol), pyridine (0.1 mL), DMF (5 mL), and a stir bar were added to a 15-mL round bottom flask outfitted with a reflux condenser under a stream of nitrogen. The reaction was heated to reflux and allowed to stir overnight (16 h). UV-vis spectroscopy was used to confirm product formation. The reaction was diluted with water and the resulting precipitate was collected by vacuum filtration. The solid was purified by column chromatography (silica gel, chloroform: hexanes 1:1). The product fractions were concentrated under reduced pressure to give the product as a red solid (51 mg, 98%). X-ray quality crystals were grown by layering MeCN over the product dissolved in CHCl₃. HRMS (MALDI) m/z: [M+H]⁺ Calcd for C₁₀₄H₉₃CuN₄Si₄⁺ 1573.5801; Found 1573.5765; μ_{eff} (Evans', CDCl₃): 1.94 μ_{B}; UV/Vis (CHCl₃) λ_{abs} (log ε): 414 (sh), 436 (5.49), 557 (4.25).

Synthesis of Compound 2-4c 5,10,15,20-tetrakis(2,6-diphenyl-4-(trimethylsilyl)phenyl)porphyrinatopalladium(II). Compound 2-2a (52 mg, 0.034 mmol), palladium(II) acetate (5.7 mg, 0.033 mmol), 2,6-lutidine (0.1 mL), 1,2,4-TCB (5 mL), and a stir bar were added to a 15 mL round bottom flask outfitted with a reflux condenser under a stream of nitrogen. The reaction was heated to reflux and allowed to stir overnight (16 h). UV-vis spectroscopy was used to confirm product formation. The reaction was diluted with water and the resulting precipitate was collected by vacuum filtration. The solid was purified by column chromatography (silica gel, chloroform: hexanes ramp chloroform to 1:1). The product fractions were concentrated under reduced pressure to give the product as a red purple solid (13 mg, 25%). X-ray quality crystals were grown by layering MeCN over the product dissolved in CHCl₃. ¹H NMR (500 MHZ, CDCl₃) δ8.33 (s, 8H), 7.76 (s, 8H), 6.50 (d, J=7.8 Hz, 16H), 6.40 (t, J=7.3 Hz, 8H), 6.21 (t, J=7.6 Hz, 16H), 0.49 (s, 36H). ¹³C{H} NMR (126 MHz, CDCl3) δ144.54, 142.28, 141.38, 133.55, 130.56, 129.33, 126.60, 125.22, 117.86, −0.63; HRMS (MALDI) m/z: [M+H]⁺ Calcd for C₁₀₄H₉₃N₄PdSi₄⁺ 1616.5544; Found 1616.5521; UV/Vis (CHCl₃) λ_{abs} (log ε): 437 (5.2), 532 (sh), 541 (4.11), 573 (2.94).

Synthesis of Compound 2-4d 5,10,15,20-tetrakis(2,6-diphenyl-4-(trimethylsilyl) phenyl) porphyrinatocobalt(II). Compound 2-2a (100 mg, 0.0662 mmol), 10 equiv of 2,6-lutidine (0.662 mmol), and 100 equiv of CoCl₂ hexahydrate (6.62 mmol) were dissolved in 1,2,4-TCB (5 mL) in a 20 mL reaction vial fitted with a pressure relief cap. The reaction mixture was heated at 213° C. without the cap for 30 min to remove water. The reaction was then sealed and allowed to stir for 2 h. The crude reaction mixture was diluted with hexanes (50 mL), wet loaded onto a silica column, and purified by normal phase flash chromatography. The product was eluted in a 1:1 solvent mixture of hexanes: chloroform and concentrated under reduced pressure to yield the isolated product as a red solid (96 mg, 91%). X-ray quality crystals were grown by layering MeCN over the product in CHCl₃ to give red plates. HRMS (MALDI) m/z: [M+H]⁺ Calcd for C₁₀₄H₉₃CON₄Si₄⁺ 1569.5838; Found 1569.5807; μ_{eff} (Evans', CDCl₃): 1.99 μ_{B}; UV/Vis (CHCl₃) λ_{abs} (log ε): 432 (5.29), 546 (4.15).

Synthesis of Compound 2-4e 5,10,15,20-tetrakis(2,6-diphenyl-4-(trimethylsilyl)phenyl)porphyrinatochloroiron(III). Compound 2-2a (73 mg, 0.0483 mmol), 10 equiv of 2,6-lutidine (0.483 mmol), and 100 equiv of FeCl₂ (4.83 mmol) were dissolved in 1,2,4-TCB (5 mL) under an aerobic atmosphere in a 20-mL reaction vial fitted with a pressure relief cap. The reaction mixture was sealed and heated to 213° C. for 1 h. The crude reaction mixture was diluted with hexanes, loaded onto a silica column, and purified by normal phase flash chromatography. The product was eluted in a 1:1 solvent mixture of hexanes: chloroform and concentrated under reduced pressure to yield the isolated product as deep purple crystals (63mg, 82%). X-ray quality crystals were grown by layering MeCN over the product dissolved in chloroform. ¹H NMR (500 MHZ, CDCl₃; paramagnetic) δ80.42 (β-pyrrole); μ_{eff} (Evans', CDCl₃): 5.65 μ_{B}; UV/Vis (CHCl₃) λ_{abs} (log ε): 361 (4.56), 372 (sh), 444 (5.17), 552 (3.86), 579(3.72), 593 (sh), 678 (sh), 707 (3.61); HRMS (MALDI) m/z: [M+H]⁺ Calcd for C₁₀₄H₉₃ClFeN₄Si₄⁺ 1601.5543; Found 1601.5447; [M−Cl]⁺ Calcd for C₁₀₄H₉₂FeN₄Si₄⁺ 1565.5776; Found 1565.5761.

Synthesis of Compound 2-4f 5,10,15,20-tetrakis(2,6-di(3,5-dimethyl)phenyl-4-(trimethylsilyl)phenyl)porphyrinatochloroiron(III). Compound 2-2d (49 mg, 0.0281 mmol), 10 equiv of 2,6-lutidine (0.281 mmol), and 100 equiv of iron(II) chloride (1.40 mmol) were dissolved in 1,2,4-TCB (5 mL) under an aerobic atmosphere in a 20 mL reaction vial fitted with a pressure relief cap. The reaction mixture was sealed and heated to 213° C. for 6 h. The crude reaction mixture was diluted with hexanes, wet loaded onto a silica column, and purified by normal phase flash chromatography. The product was eluted in a 1:1 solvent mixture of hexanes: chloroform and concentrated under reduced pressure to yield the isolated product as deep purple crystals (36.2 mg, 71%). X-ray quality crystals were grown by layering MeCN over the product dissolved in toluene. HRMS (MALDI) m/z: [M+H]⁺ Calcd for C₁₂₀H₁₂₅ClFeN₄Si₄⁺ 1825.8047; Found 1825.8016; [M−Cl]⁺ Calcd for C₁₂₀H₁₂₄FeN₄Si₄⁺ 1789.8280; Found 1789.8306; UV/Vis (CHCl₃) λ_{abs} (log ε): 379 (4.45), 442 (5.03), 520 (4.13), 588 (3.62), 707 (3.61). ¹H NMR (500 MHz, CDCl₃; paramagnetic) δ81.06 (β-pyrrole); μ_{eff} (Evans', CDCl₃): 6.59 μ_{B}

X-ray crystallography. Crystals of 2-2b, 2-2c, 2-2d, 2-2e, 2-2f, 2-2h, 2-2i, 2-2k, 2-2l, 2-2m, 2-2o, 2-2p, 2-2q, 3-2b, 2-4a, 2-4b, 2-4c, 2-4d, 2-4e, and 2-4f were grown as described above. Single crystals suitable for X-ray diffraction were selected under a microscope, loaded onto a nylon fiber loop using Paratone-N, and mounted onto a Rigaku XtaLAB Synergy-S single-crystal diffractometer. Each crystal was cooled to 100 K under a stream of nitrogen. Diffraction of Cu Kα radiation from a PhotonJet-S microfocus source was detected using a HyPix-6000HE hybrid photon counting detector. Screening, indexing, data collection, and data processing were performed with CrysAlis^{Pro}.³ The structures were solved using SHELXT and refined using SHELXL as implemented in OLEX2 following established strategies.⁴⁻⁷ Unless otherwise specified in the CIF all non-H atoms were refined anisotropically and H atoms were placed at calculated positions and refined with a riding model and coupled isotropic displacement parameters. As noted in the appropriate CIFs, a number of the structures featured pockets of disordered solvent that could not be satisfactorily modeled. In these instances, the contribution of the electron density in those pockets to the observed structure factors was masked using Olex2. Refinement parameters are collected in Tables 2-1 to 2-7.
Pocket volume estimation. Pocket volumes were calculated using POVME2. PDB files of each porphyrin were generated from the corresponding X-ray diffraction coordinates. The grid spacing was set to 0.5 Å and a points-inclusion sphere of 10-Å radius was generated at the center of each porphyrin. A contiguous pocket-seed sphere of 4-Å radius was generated at the center of each porphyrin and a contiguous points criterion of 5 was employed (criteria of 3 and 7 were used for 2-2b and 2-2m, respectively). Molecular graphics were generated with UCSF ChimeraX.² Pocket volumes are collected in Table 2-8.

TABLE 2-1
Crystallographic Refinement Details

| Compound | 2-2b•2MeCN | 2-2c•MeCN•1.5CHCl₃ | 2-2d•MeCN•3C₆H₃Cl₃ |
|---|---|---|---|
| Formula | C₁₀₈H₈₄F₁₆N₆Si₄ | C_{107.5}H_{82.5}C1_{20.5}N₅Si₄ | C₁₄₀H₁₃₈Cl₉N₅Si₄ |
| FW | 1882.17 | 2283.37 | 2321.96 |
| T (K) | 100.0(11) | 99.99(10) | 100.01(10) |
| λ (Å) | 1.54184 | 1.54184 | 1.54184 |
| Crystal | Triclinic | Monoclinic | Monoclinic |
| System | | | |
| Space group | P1̅ | I2/a | P2₁/n |
| a (Å) | 12.5909(2) | 23.2819(2) | 16.53020(10) |
| b (Å) | 13.6056(2) | 19.67030(10) | 40.0808(3) |
| c (Å) | 17.3122(2) | 47.5453(3) | 19.68380(10) |
| α (°) | 103.7060(10) | 90 | 90 |
| β (°) | 106.6640(10) | 92.3390(10) | 97.4540(10) |
| γ (°) | 106.8540(2) | 90 | 90 |
| Volume | 2548.03(7) | 21755.8(3) | 12931.17(14) |
| (Å³) | | | |
| Z | 1 | 8 | 4 |
| Size | 0.16 × 0.07 × 0.04 | 0.41 × 0.31 × 0.03 | 0.16 × 0.11 × 0.06 |
| (mm³) | | | |
| θ range (°) | 2.843-67.078 | 2.942-67.077 | 2.518-67.079 |
| Total data | 34597 | 138551 | 175824 |
| Unique data | 9086 | 19392 | 23102 |
| Parameters | 610 | 1307 | 1599 |
| Completeness | 99.9 | 99.9 | 100.0 |
| (%) | | | |
| Rᵢₙₜ (%) | 2.90 | 4.61 | 4.19 |
| R₁ | 3.69 | 6.39 | 7.74 |
| (%, I > 2σ) | | | |
| R₁ | 4.14 | 6.90 | 8.05 |
| (%, all data) | | | |
| wR₂ | 9.60 | 18.20 | 20.51 |
| (%, I > 2σ) | | | |
| wR₂ | 9.87 | 18.72 | 20.70 |
| (%, all data) | | | |
| S | 1.028 | 1.033 | 1.158 |

TABLE 2-2
Crystallographic Refinement Details

| Compound | 2-2e•MeCN•C₇H₈ | 2-2f•2MeCN | 2-2h•2MeCN•C₇H₅N | |
|---|---|---|---|---|
| Formula | C₁₂₃H₁₂₄N₆Si₄ | C₁₃₂H₁₄₈N₆Si₄ | C₁₁₅H₉₇F₈N₇Si₄ | |
| FW | 1798.63 | 1930.92 | 1841.35 | |
| T (K) | 100.6(10) | 100.00(11) | 101 | (1) |
| λ (Å) | 1.54184 | 1.54184 | 1.54184 | |
| Crystal | Monoclinic | Triclinic | Monoclinic | |
| System | | | | |
| Space group | P2₁/c | P1̅ | P2₁/c | |
| a (Å) | 16.34530(10) | 13.3916(5) | 17.9444 | (2) |
| b (Å) | 29.9212(2) | 15.4829(7) | 14.42580 | (10) |
| c (Å) | 23.1963(2) | 16.1203(6) | 23.4254 | (2) |
| α (°) | 90 | 67.333(4) | 90 | |
| β (°) | 107.5830(10) | 72.696(3) | 110.6190 | (10) |
| γ (°) | 90 | 87.191(3) | 90 | |
| Volume | 10814.62(15) | 2936.5(2) | 5675.51 | (10) |
| (Å³) | | | | |

| | | | |
|---|---|---|---|
| Z | 4 | 1 | 2 |
| Size | 0.13 × 0.08 × 0.07 | 0.88 × 0.18 × 0.05 | 0.29 × 0.17 × 0.05 |
| (mm³) | | | |
| θ range (°) | 2.485-67.079 | 3.117-67.067 | 2.631-67.078 |
| Total data | 171800 | 38820 | 106189 |
| Unique data | 19313 | 10456 | 10128 |
| Parameters | 1281 | 732 | 646 |
| Completeness | 100 | 99.9 | 99.9 |
| (%) | | | |
| Rᵢₙₜ (%) | 3.28 | 5.87 | 3.90 |
| R₁ | 4.51 | 5.63 | 5.65 |
| (%, I > 2σ) | | | |
| R₁ | 4.79 | 6.59 | 5.86 |
| (%, all data) | | | |
| wR₂ | 11.01 | 15.16 | 15.41 |
| (%, I > 2σ) | | | |
| wR₂ | 11.16 | 15.97 | 15.57 |
| (%, all data) | | | |
| S | 1.114 | 1.033 | 1.047 |

TABLE 2-3
Crystallographic Refinement Details

| Compound | 2-2i•CHCl₃ | 2-2k•2MeCN•CHCl₃ | 2-2l•2MeCN |
|---|---|---|---|
| Formula | C₁₁₃H₈₇Cl₃F₂₄N₄Si₄ | C₁₁₇H₁₁₇Cl₃N₆O₈Si₄ | C₁₄₀H₁₁₆N₆Si₄ |
| FW | 2175.57 | 1953.87 | 1994.74 |
| T (K) | 99.99(10) | 100.0(13) | 99.98(15) |
| λ (Å) | 1.54184 | 1.54184 | 1.54184 |
| Crystal | Monoclinic | Triclinic | Monoclinic |
| System | | | |
| Space group | P2₁/n | P1̅ | P2₁/c |
| a (Å) | 18.7167(2) | 11.9189(2) | 16.6762(3) |
| b (Å) | 23.8213(2) | 15.7967(3) | 23.8081(4) |
| c (Å) | 26.6679(3) | 16.5347(3) | 30.0004(3) |
| α (°) | 90 | 101.9810(10) | 90 |
| β (°) | 93.2670(10) | 108.897(2) | 91.4430(10) |
| γ (°) | 90 | 107.1330(10) | 90 |
| Volume | 11870.7(2) | 2651.88(9) | 11907.2(3) |
| (Å³) | | | |
| Z | 4 | 1 | 4 |
| Size | 0.22 × 0.1 × 0.06 | 0.23 × 0.09 × 0.06 | 0.23 × 0.15 × 0.03 |
| (mm³) | | | |
| θ range (°) | 2.489-67.077 | 2.998-67.078 | 2.369-67.078 |
| Total data | 167793 | 35890 | 95662 |
| Unique data | 21182 | 9457 | 21185 |
| Parameters | 1510 | 651 | 1598 |
| Completeness | 100.0 | 99.9 | 99.6 |
| (%) | | | |
| Rᵢₙₜ (%) | 4.76 | 3.42 | 3.62 |
| R₁ | 6.51 | 6.01 | 6.76 |
| (%, I > 2σ) | | | |
| R₁ | 7.53 | 6.66 | 8.32 |
| (%, all data) | | | |
| wR₂ | 16.97 | 16.44 | 18.24 |
| (%, I > 2σ) | | | |
| wR₂ | 17.69 | 16.97 | 19.37 |
| (%, all data) | | | |
| S | 1.029 | 1.042 | 1.022 |

TABLE 2-4
Crystallographic Refinement Details

| Compound | 2-2m | 2-2o•5MeCN | 2-2p |
|---|---|---|---|
| Formula | C₈₀H₉₄N₄Si₄ | C₁₃₈H₁₇₃N₉Si₁₂ | C₁₂₈H₁₂₆N₄O₁₆Si₄ |
| FW | 1223.95 | 2294.92 | 2088.68 |
| T (K) | 99.99(14) | 100.01(10) | 100.01(10) |
| λ (Å) | 1.54184 | 1.54184 | 1.54184 |
| Crystal | Triclinic | Monoclinic | Monoclinic |
| System | | | |
| Space group | P1̅ | P2₁/n | I2/a |
| a (Å) | 14.3294(2) | 23.9843(2) | 22.8311(2) |
| b (Å) | 16.7136(3) | 24.2555(2) | 24.7546(3) |
| c (Å) | 18.2148(2) | 27.5665(2) | 23.6019(4) |
| α (°) | 103.3400(10) | 90 | 90 |
| β (°) | 90.2160(10) | 90.8630(10) | 98.1630(10) |
| γ (°) | 100.0870(10) | 90 | 90 |
| Volume | 4174.61(11) | 16035.0(2) | 13204.0(3) |
| (Å³) | | | |
| Z | 2 | 4 | 4 |
| Size | 0.29 × 0.08 × 0.03 | 0.81 × 0.6 × 0.27 | 0.29 × 0.25 × 0.18 |
| (mm³) | | | |
| θ range (°) | 2.496-67.075 | 2.424-67.080 | 2.601-67.073 |
| Total data | 56459 | 218940 | 11799 |
| Unique data | 14868 | 28618 | 11799 |
| Parameters | 897 | 1681 | 878 |
| Completeness | 99.8 | 99.9 | 100.0 |
| (%) | | | |
| Rᵢₙₜ (%) | 4.38 | 5.03 | N/A^{a} |
| R₁ | 8.19 | 6.82 | 10.13 |
| (%, I > 2σ) | | | |
| R₁ | 8.84 | 7.44 | 10.63 |
| (%, all data) | | | |
| wR₂ | 20.86 | 18.58 | 30.79 |
| (%, I > 2σ) | | | |
| wR₂ | 21.25 | 19.11 | 31.29 |
| (%, all data) | | | |
| S | 1.091 | 1.024 | 1.049 |

^{a}Non-merohedral twin

TABLE 2-5
Crystallographic Refinement Details

| Compound | 2-2q•½Et₂O | 2-3b•5C₆H₁₄O₃ | 2-4a•MeCN•H₂O |
|---|---|---|---|
| Formula | C₉₀H₉₉N₂₀O_{0.50}Si₄ | C₁₂₂H₁₁₂F₁₆N₄Na₄O₂₇S₄ | C₁₀₆H₉₇N₅OSi₄Zn |
| FW | 1581.25 | 2590.35 | 1634.61 |
| T (K) | 100.0(3) | 100.01(11) | 100.0(10) |
| λ (Å) | 1.54184 | 1.54184 | 1.54184 |
| Crystal | Triclinic | Monoclinic | Triclinic |
| System | | | |
| Space group | P1̅ | C2/m | P1̅ |
| a (Å) | 15.5214(3) | 13.5375(2) | 13.1539(2) |
| b (Å) | 16.8377(3) | 32.1685(4) | 13.6553(2) |
| c (Å) | 19.6805(3) | 14.4984(2) | 16.1176(3) |
| α (°) | 104.498(2) | 90 | 73.1390(10) |
| β (°) | 91.652(2) | 90.9620(10) | 70.611(2) |
| γ (°) | 92.538(2) | 90 | 61.251(2) |
| Volume | 4970.49 | 6312.89(15) | 2364.30(8) |
| (Å³) | | | |
| Z | 2 | 2 | 1 |
| Size | 0.27 × 0.05 × 0.04 | 0.15 × 0.08 × 0.07 | 0.1 × 0.06 × 0.03 |
| (mm³) | | | |
| θ range (°) | 2.321-67.077 | 2.747-67.067 | 2.943-67.078 |
| Total data | 64662 | 39300 | 69421 |
| Unique data | 17671 | 5750 | 8457 |
| Parameters | 1121 | 537 | 552 |
| Completeness | 99.5 | 100.0 | 100 |
| (%) | | | |
| Rᵢₙₜ (%) | 6.26 | 4.87 | 3.58 |
| R₁ | 6.63 | 4.88 | 5.65 |
| (%, I > 2σ) | | | |
| R₁ | 7.46 | 5.22 | 5.95 |
| (%, all data) | | | |
| wR₂ | 18.38 | 13.17 | 14.83 |
| (%, I > 2σ) | | | |
| wR₂ | 19.22 | 13.43 | 15.04 |
| (%, all data) | | | |
| S | 1.041 | 1.050 | 1.115 |

TABLE 2-6
Crystallographic Refinement Details

| Compound | 2-4b•2MeCN | 2-4c•2MeCN | 2-4d•2MeCN |
|---|---|---|---|
| Formula | C₁₀₈H₉₈CuN₆Si₄ | C₁₀₈H₉₈N₆PdSi₄ | C₁₀₈H₉₈CoN₆Si₄ |
| FW | 1655.82 | 1698.68 | 1651.21 |
| T (K) | 100.0(10) | 99.9(2) | 101(2) |
| λ (Å) | 1.54184 | 1.54184 | 1.54184 |
| Crystal | Triclinic | Triclinic | Triclinic |
| System | | | |
| Space group | P1̅ | P1̅ | P1̅ |
| a (Å) | 13.1905(2) | 13.2012(3) | 13.1816(2) |
| b (Å) | 13.6409(3) | 13.6615(3) | 13.6278(2) |
| c (Å) | 16.0599(2) | 16.0477(3) | 16.0608(2) |
| α (°) | 72.320(2) | 72.357(2) | 72.3800(10) |
| β (°) | 71.031(2) | 70.882(2) | 70.9320(10) |
| γ (°) | 61.419(2) | 61.519(2) | 61.282(2) |
| Volume | 2361.21(9) | 2365.70(10) | 2354.14(7) |
| (Å³) | | | |
| Z | 1 | 1 | 1 |
| Size | 0.15 × 0.07 × 0.02 | 0.42 × 0.16 × 0.04 | 0.1 × 0.08 × 0.03 |
| (mm³) | | | |
| θ range (°) | 2.957-67.073 | 2.961-67.080 | 2.957-67.076 |
| Total data | 66105 | 63611 | 31966 |
| Unique data | 8428 | 8428 | 8410 |
| Parameters | 545 | 545 | 545 |
| Completeness | 99.9 | 99.6 | 99.9 |
| (%) | | | |
| Rᵢₙₜ (%) | 4.61 | 5.48 | 3.01 |
| R₁ | 3.60 | 3.87 | 3.30 |
| (%, I > 2σ) | | | |
| R₁ | 4.14 | 4.05 | 3.42 |
| (%, all data) | | | |
| wR₂ | 9.38 | 10.30 | 7.76 |
| (%, I > 2σ) | | | |
| wR₂ | 9.70 | 10.45 | 7.82 |
| (%, all data) | | | |
| S | 1.035 | 1.047 | 1.023 |

TABLE 2-7
Crystallographic Refinement Details

| Compound | 2-4e•MeCN•CHCl₃ | 2-4f•½C₇H₈ |
|---|---|---|
| Formula | C₁₀₇H₉₆Cl₄FeN₅Si₄ | C_{123.5}H₁₂₈ClFeN₄Si₄ |
| FW | 1761.89 | 1871.95 |
| T (K) | 100.0(10) | 100.0(12) |
| λ (Å) | 1.54184 | 1.54184 |
| Crystal | Triclinic | Monoclinic |
| System | | |
| Space group | P1̅ | P2₁/n |
| a (Å) | 12.8777(3) | 13.85030(10) |
| b (Å) | 16.3497(4) | 31.0859(3) |
| c (Å) | 23.5222(4) | 26.2645(3) |
| α (°) | 106.739(2) | 90 |
| β (°) | 90.100(2) | 90.4670(10) |
| γ (°) | 90.228(2) | 90 |
| Volume | 4742.60(19) | 11307.78(19) |
| (Å³) | | |
| Z | 2 | 4 |
| Size | 0.21 × 0.09 × 0.05 | 0.07 × 0.05 × 0.03 |
| (mm³) | | |
| θ range (°) | 2.822-67.081 | 2.843-67.071 |
| Total data | 61182 | 143167 |
| Unique data | 16845 | 20048 |
| Parameters | 1158 | 1520 |
| Completeness | 99.4 | 99.2 |
| (%) | | |
| Rᵢₙₜ (%) | 4.20 | 5.84 |
| R₁ | 11.82 | 7.60 |
| (%, I > 2σ) | | |
| R₁ | 12.45 | 9.08 |
| (%, all data) | | |
| wR₂ | 26.22 | 18.59 |
| (%, I > 2σ) | | |
| wR₂ | 26.51 | 19.27 |
| (%, all data) | | |
| S | 1.222 | 1.126 |

TABLE 2-8
Pocket Volumes

| | | |
|---|---|---|
| | Pocket | Pocket Volume |
| Compound | Volume A³ (^{a}) | (top, bottom) A³ |

| | | |
|---|---|---|
| 2-2a | 22 | (11) |
| 2-2b | 23.25 | (12) |

| | | |
|---|---|---|
| 2-2c | 34.75 | (28.5, 6.25) |
| 2-2d | 12.5 | (9, 3.5) |

| | | |
|---|---|---|
| 2-2e | 19.25 | (10) |
| 2-2f | 34.75 | (17.3) |
| 2-2h | 25.0 | (12.5) |

| | | |
|---|---|---|
| 2-2i | 13.0 | (7.625, 5.375) |

| | | |
|---|---|---|
| 2-2k | 31.75 | (16) |
| 2-2l | 20.875 | (11) |
| 2-2m | 44.5 | (22) |
| 2-2o | 23.875 | (12.1) |

| | | |
|---|---|---|
| 2-2p | 16.25 | (10.75, 5.5) |
| 2-2q | 10.25 | Only top pocket |

| | | |
|---|---|---|
| 3-2b | 7.75 | (3.875) |

(^{a}) average volume across both pockets.

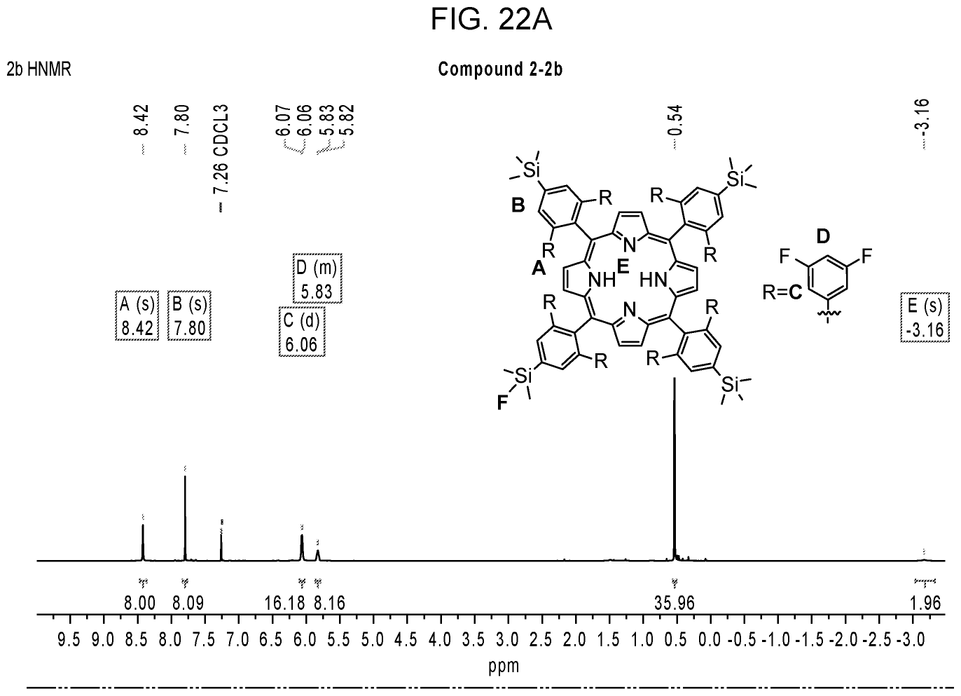

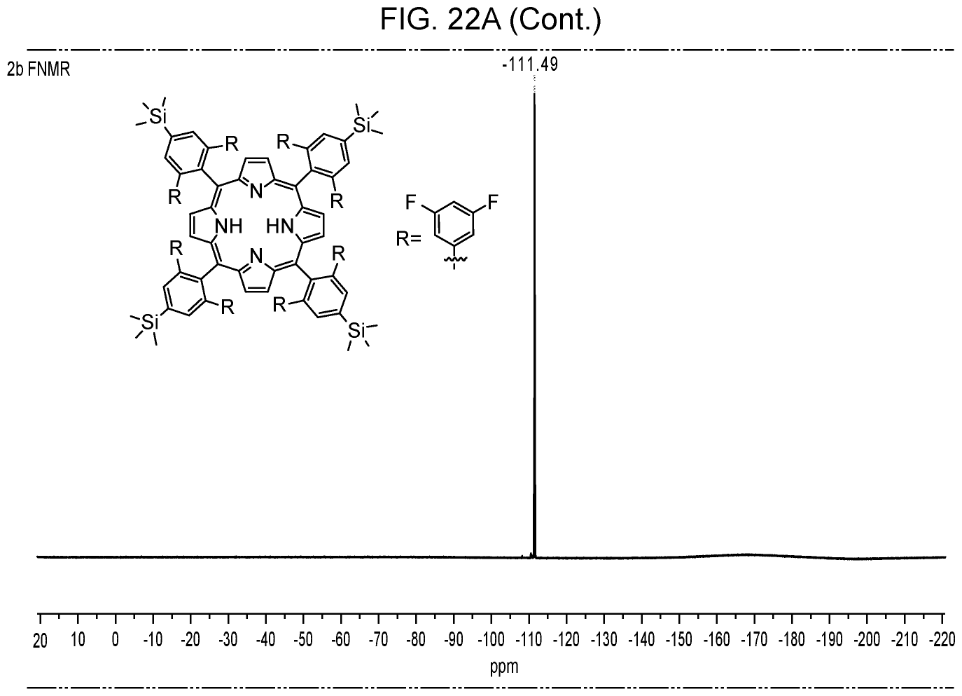

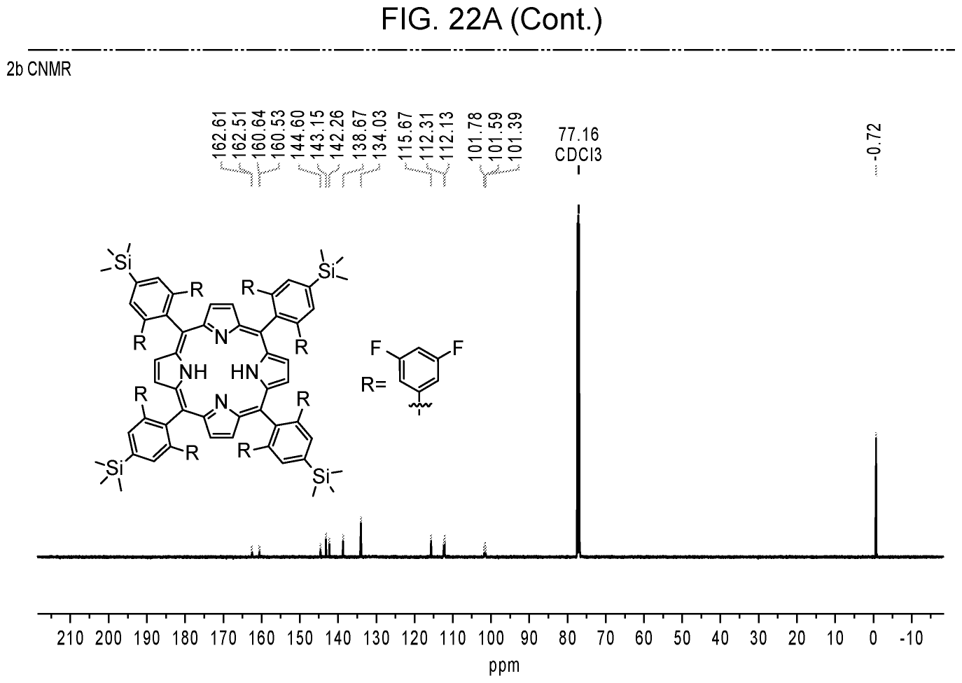

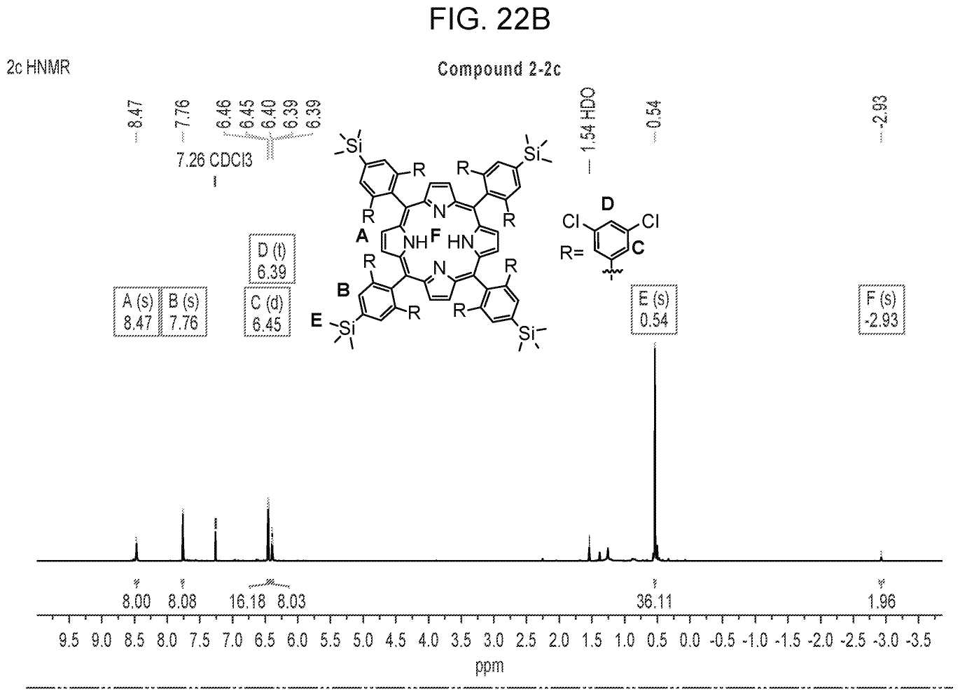

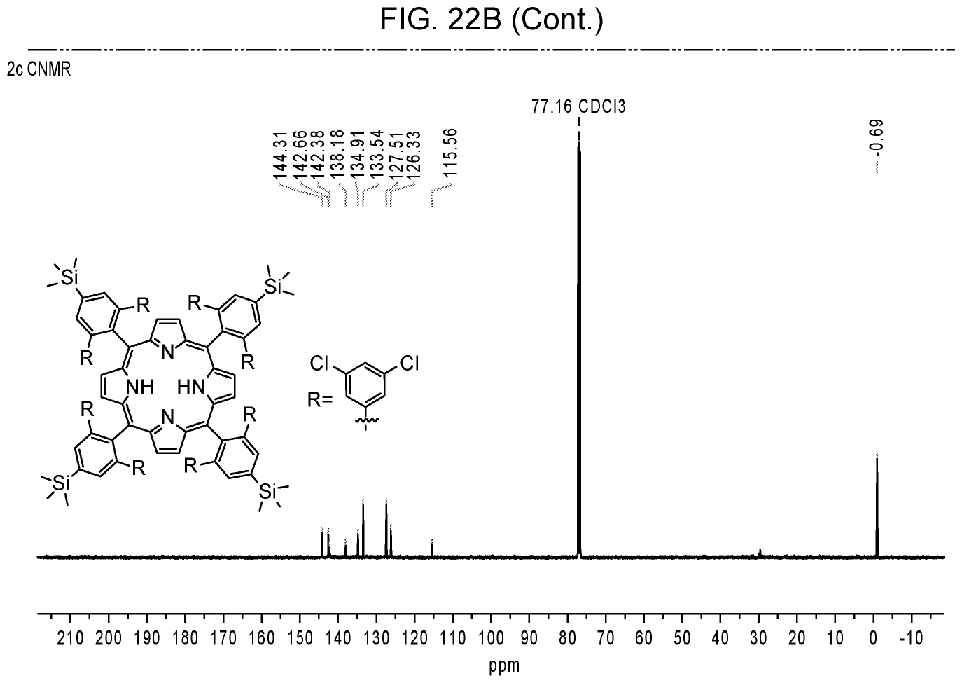

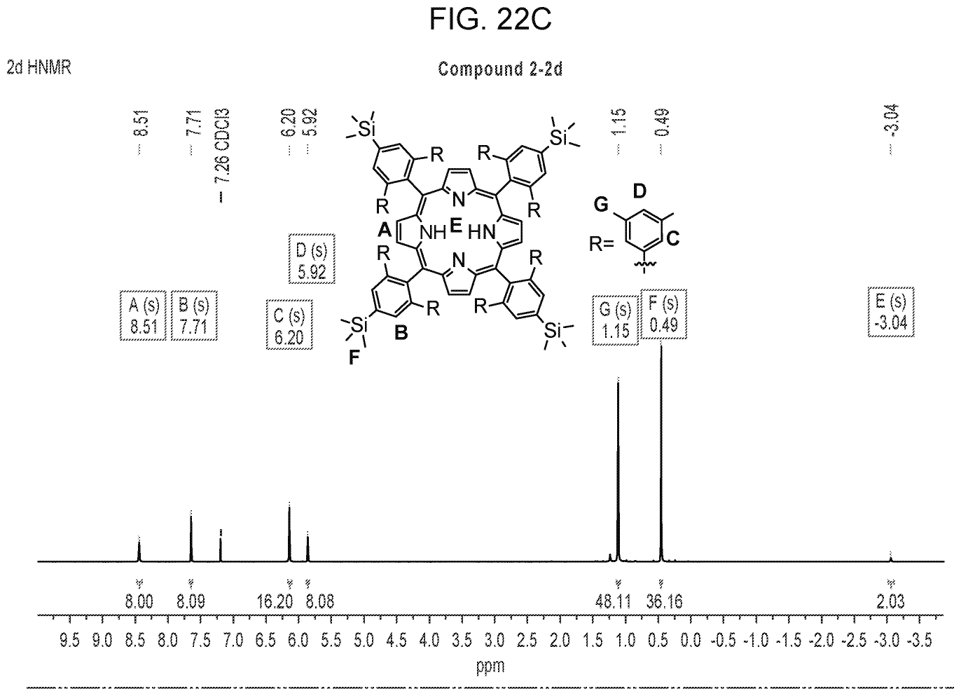

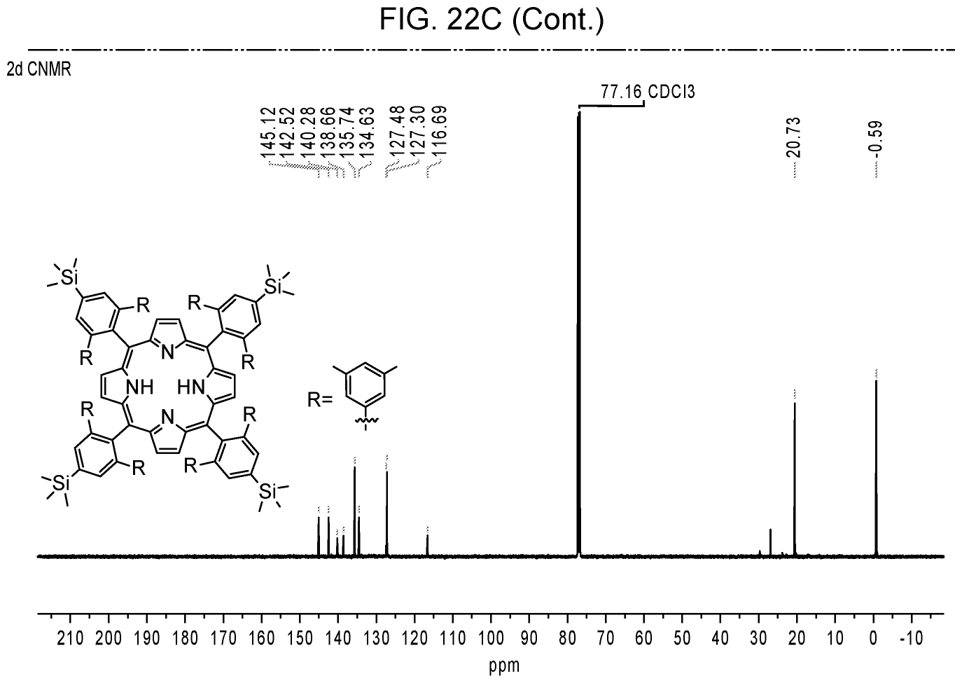

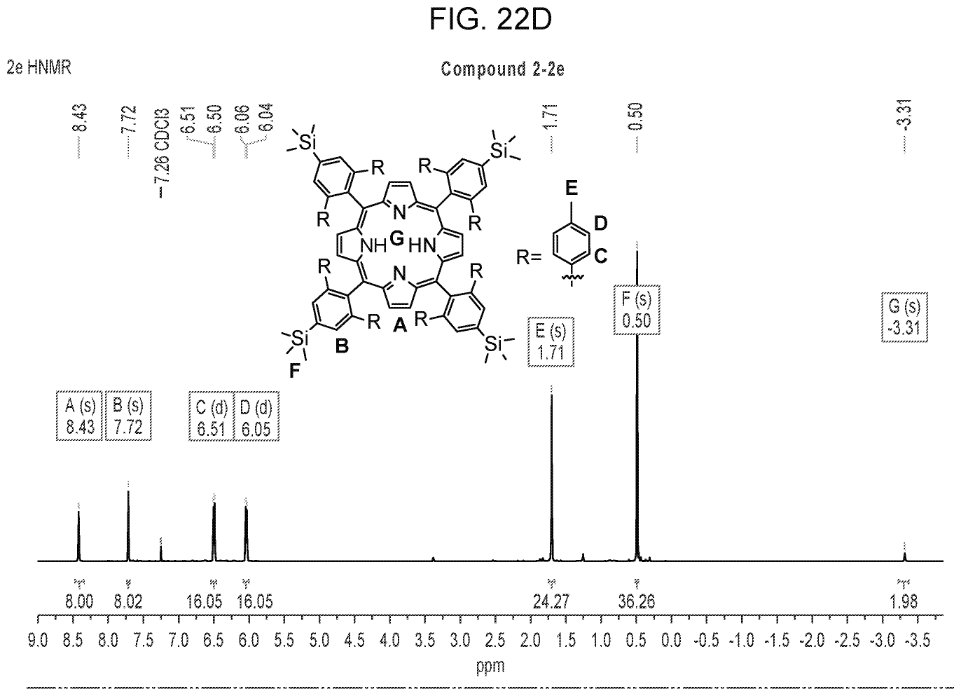

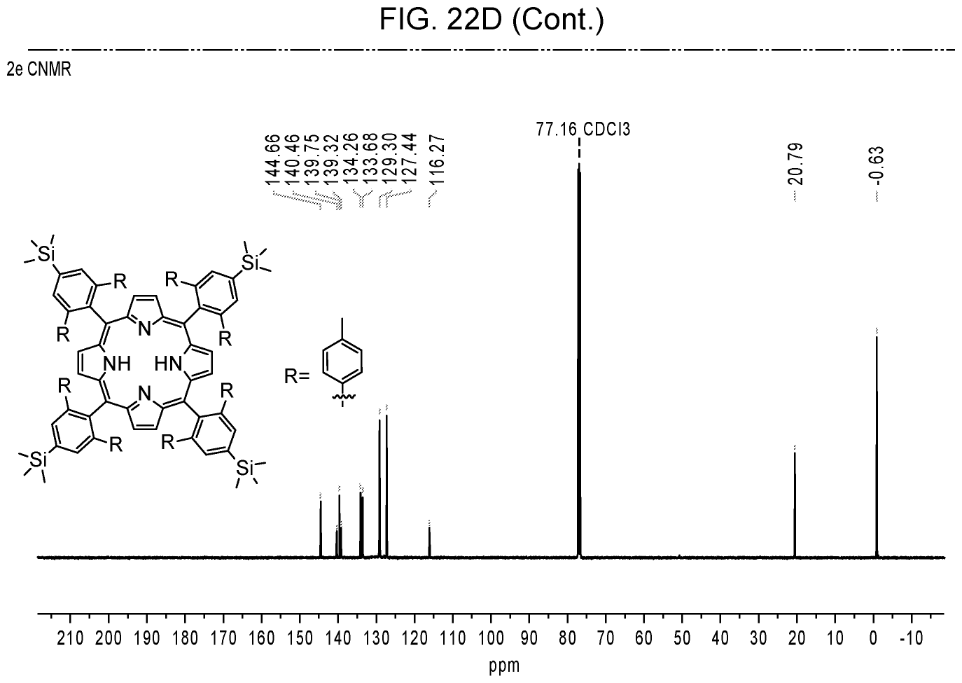

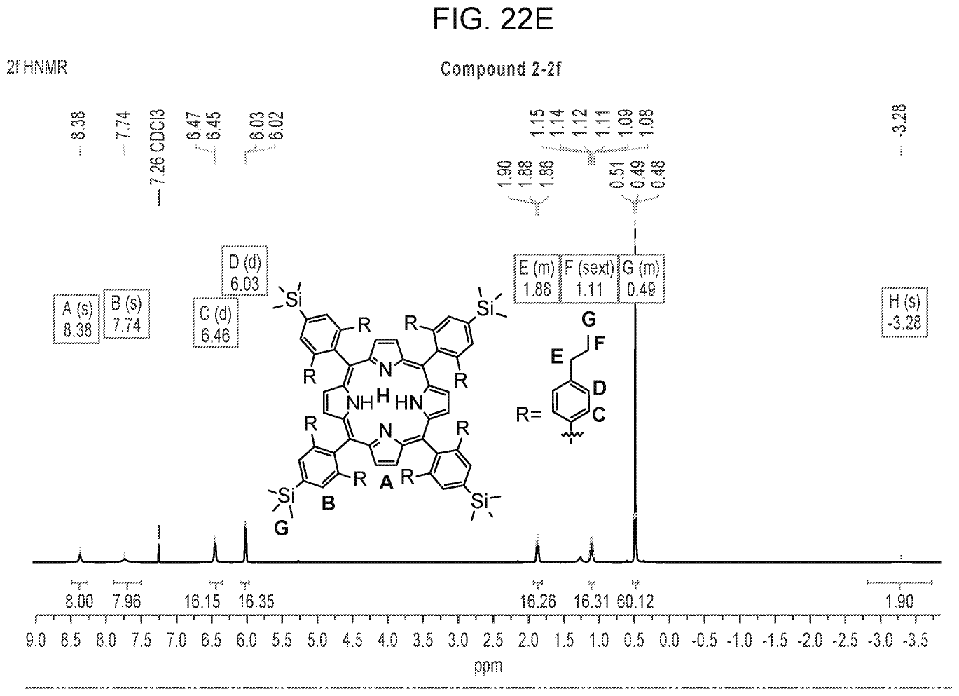

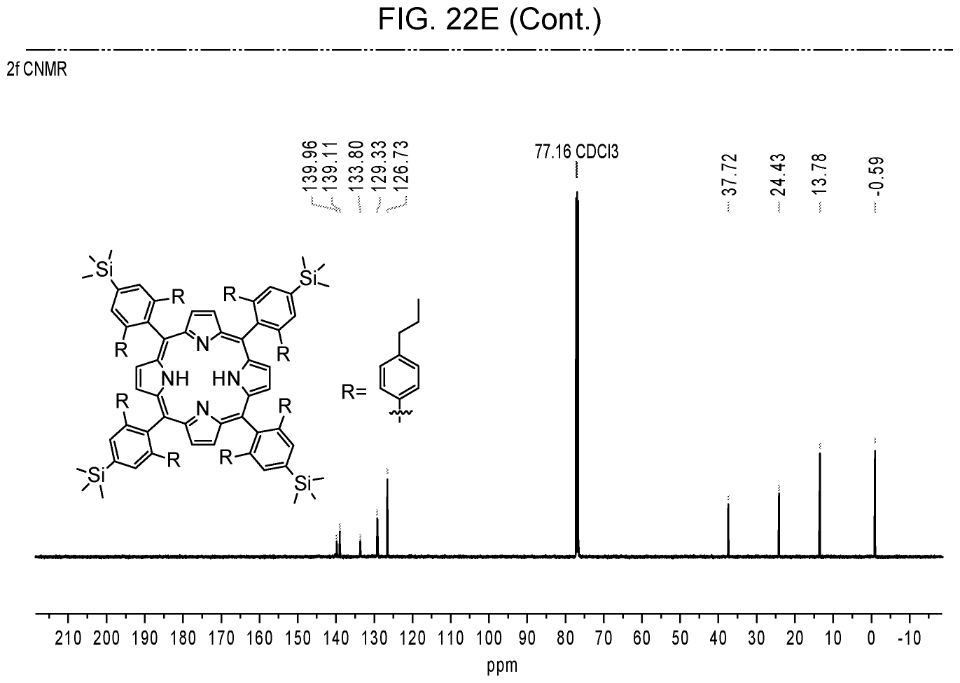

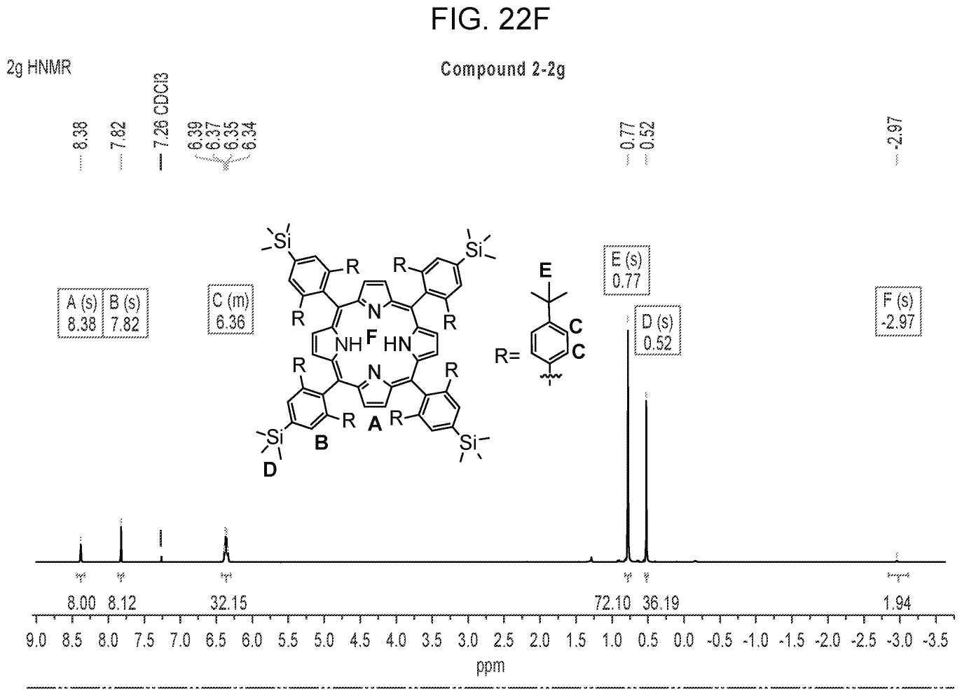

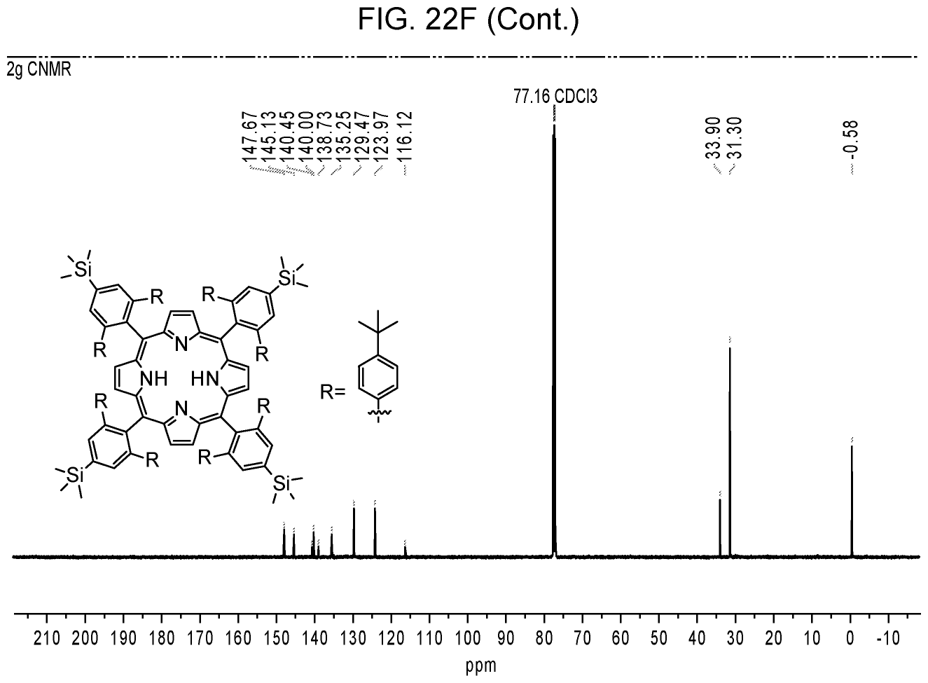

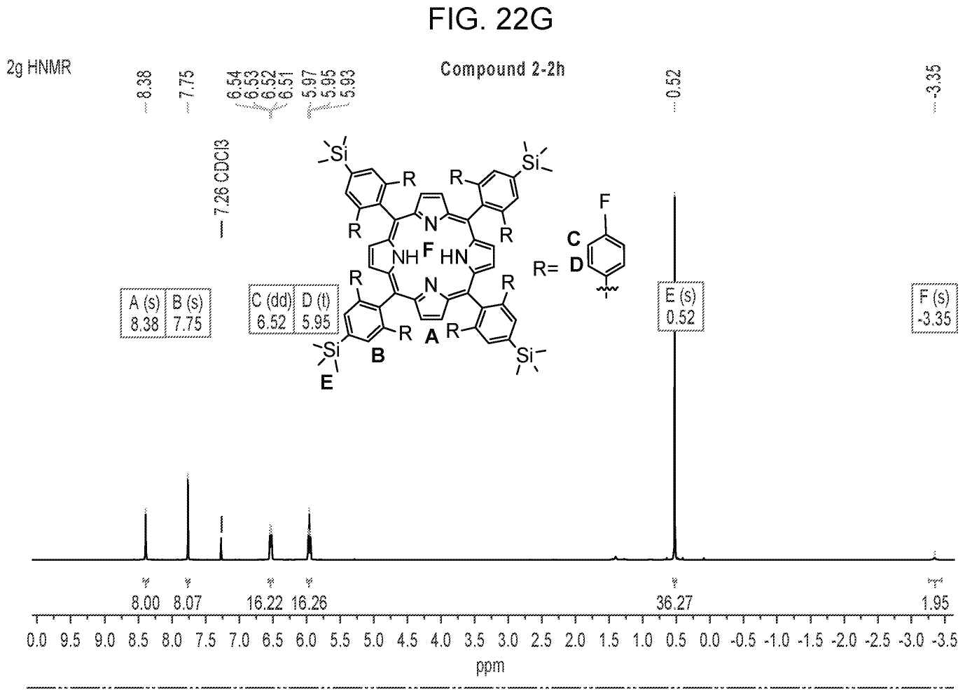

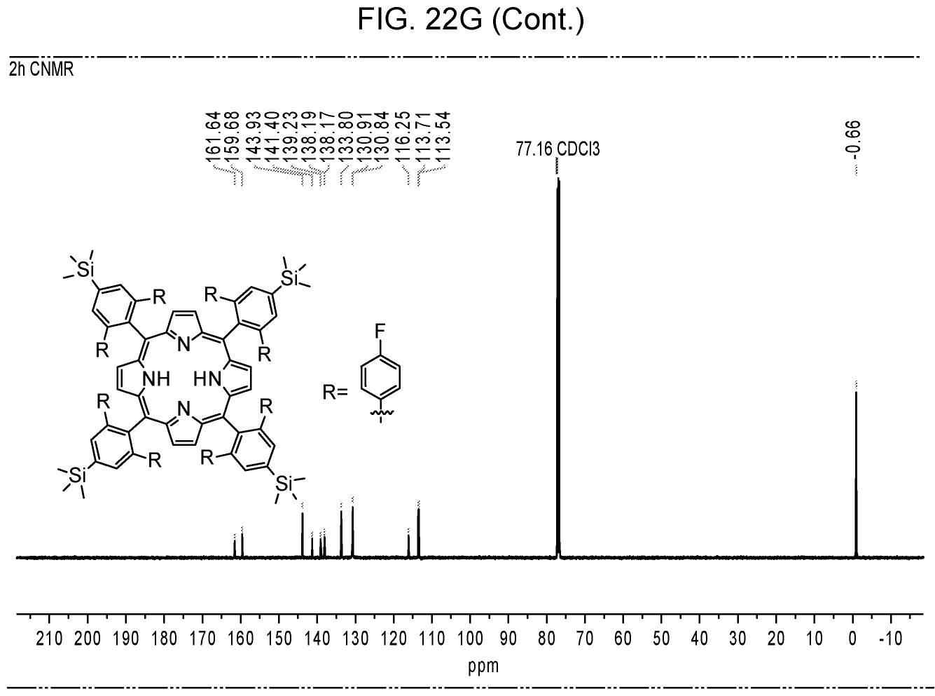

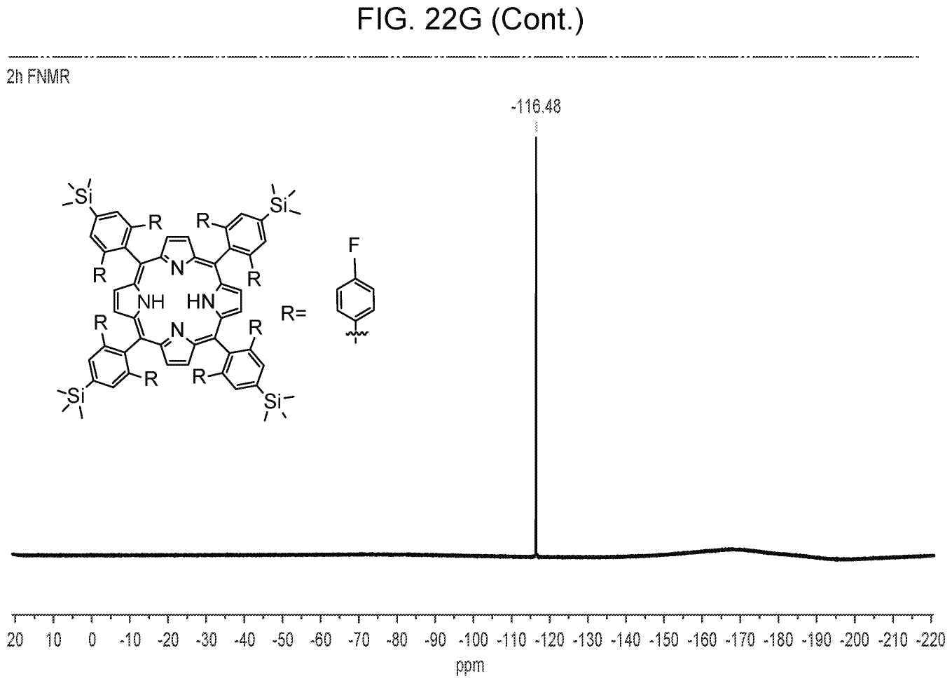

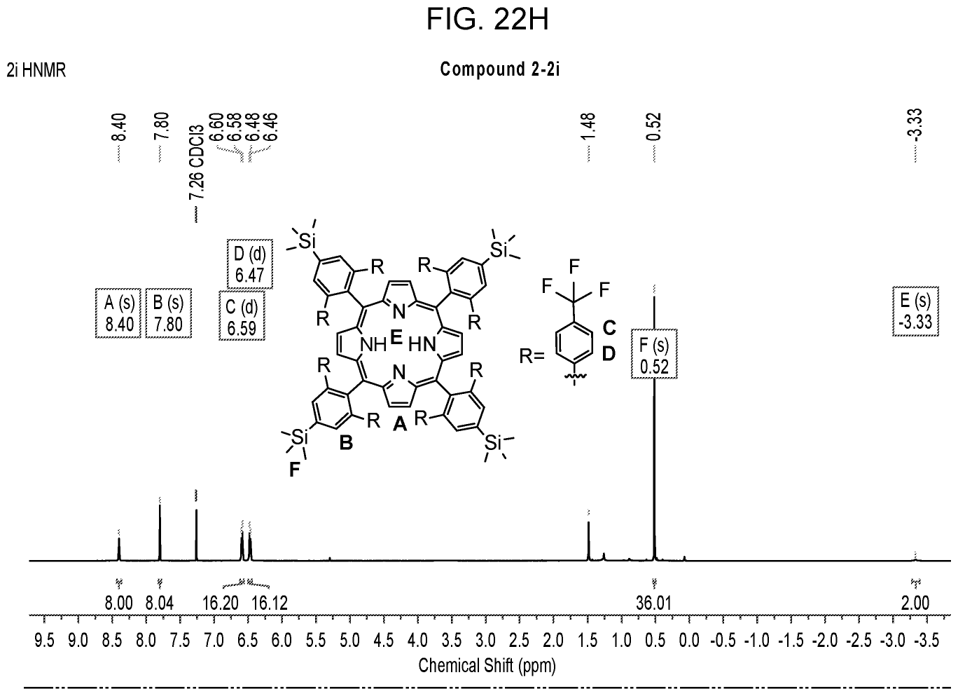

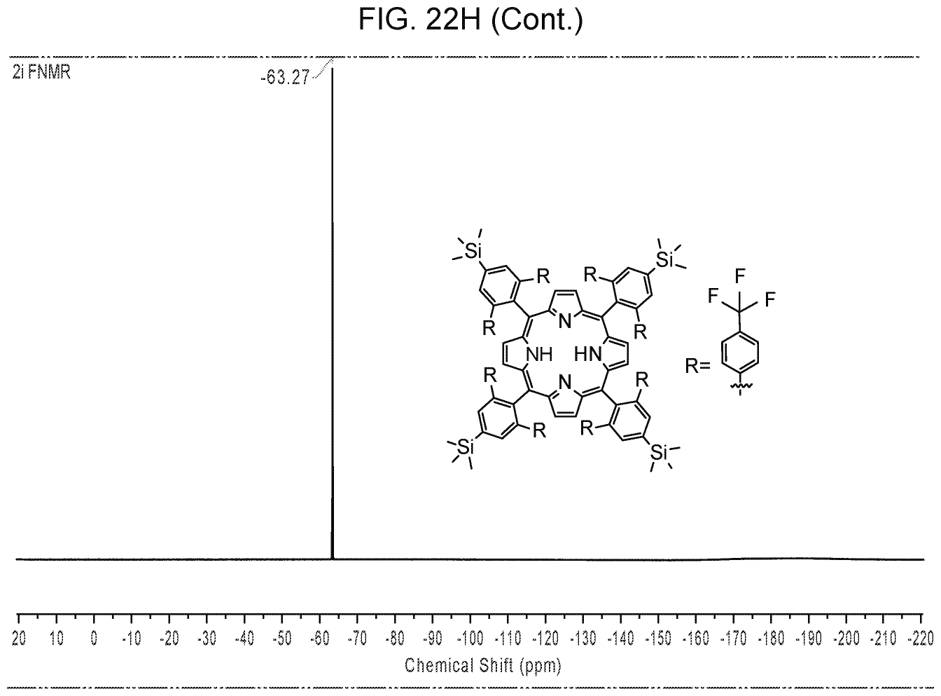

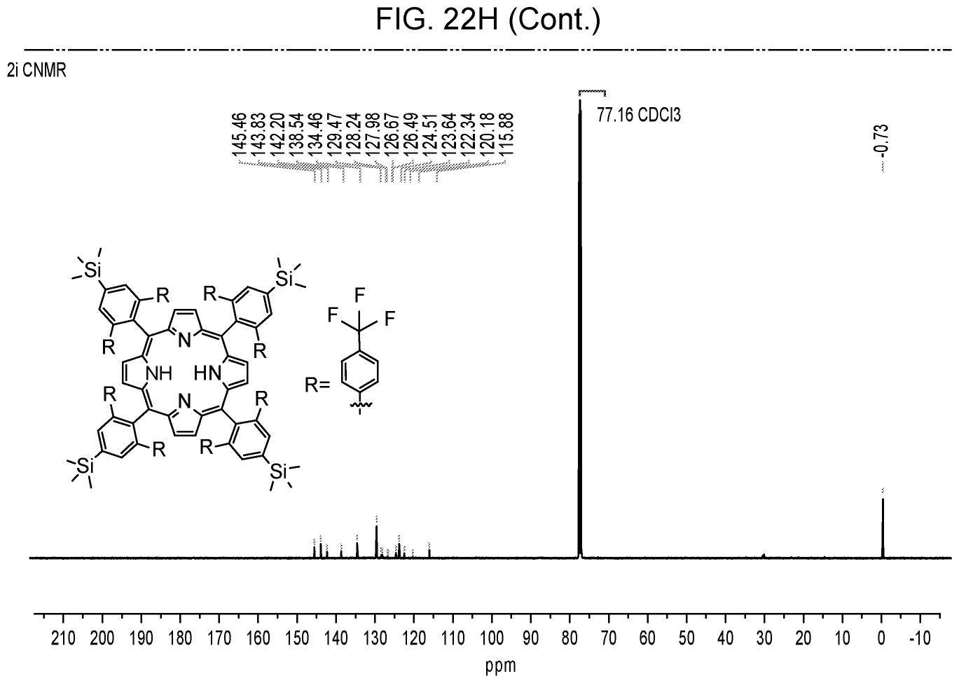

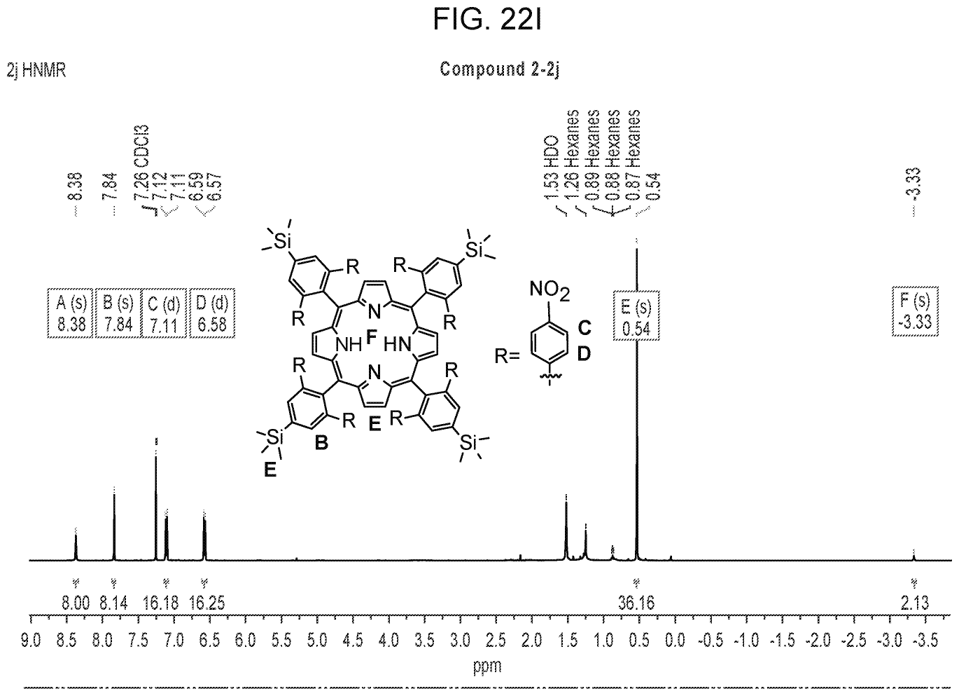

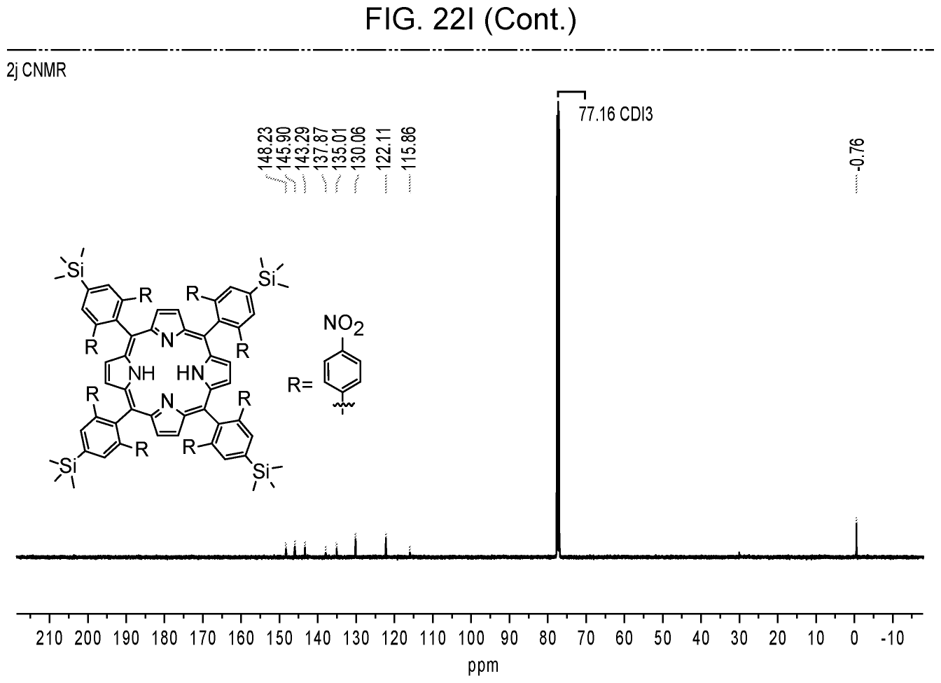

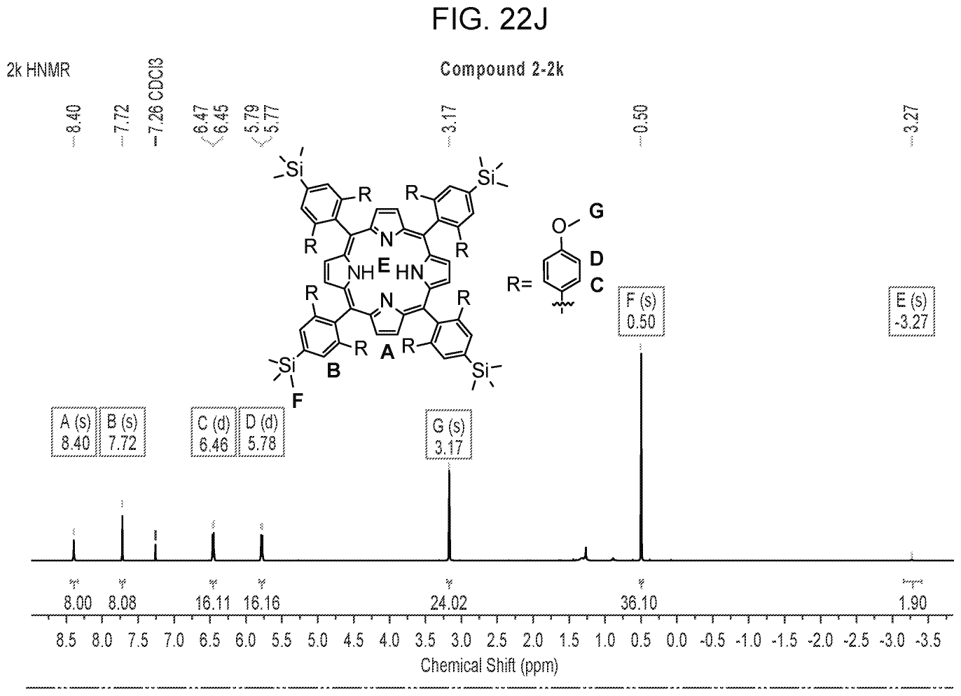

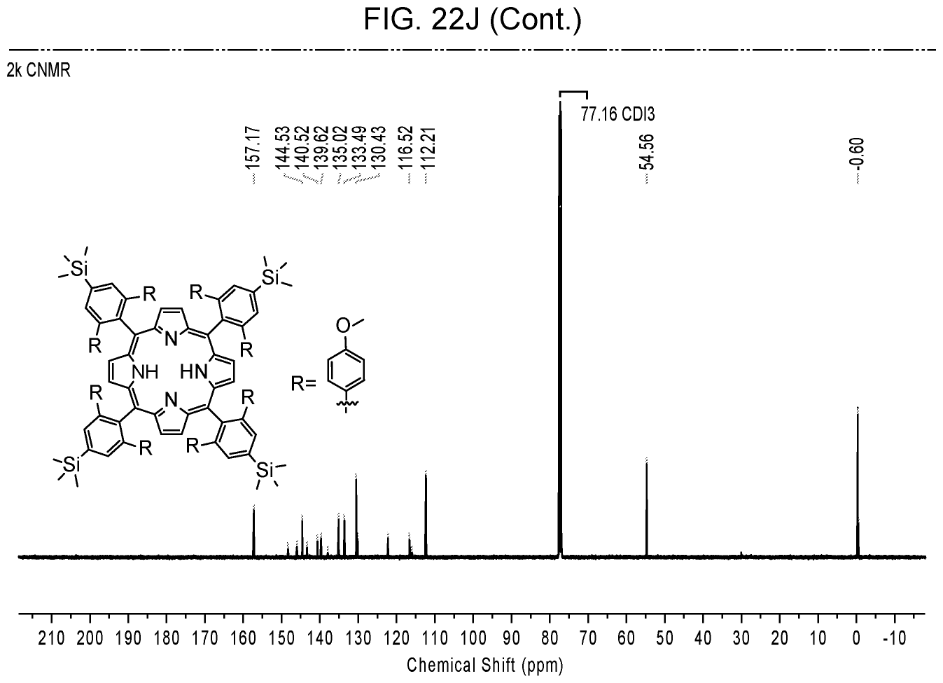

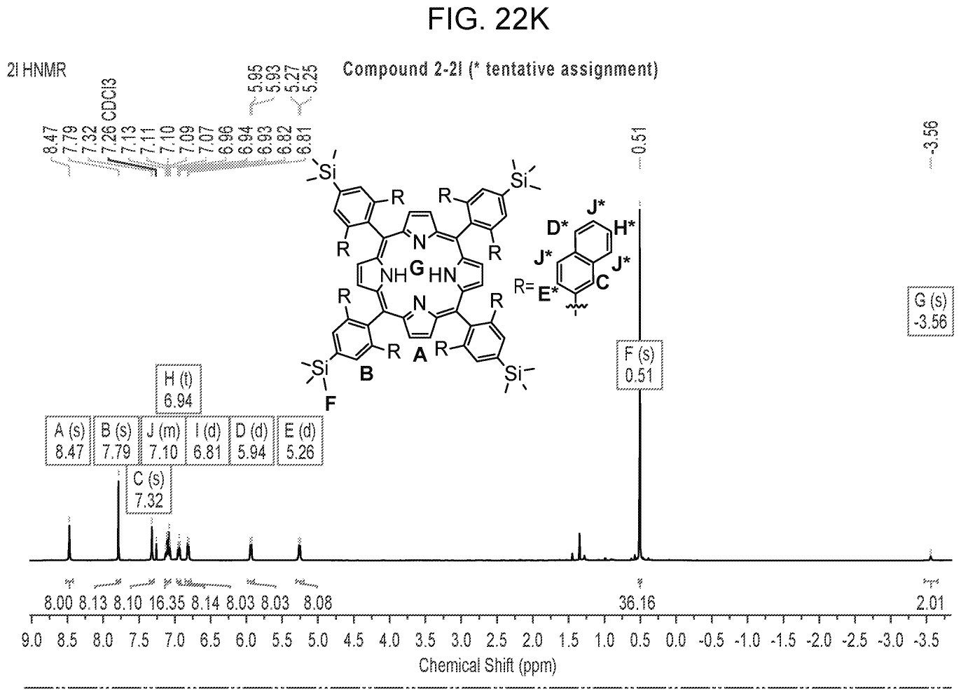

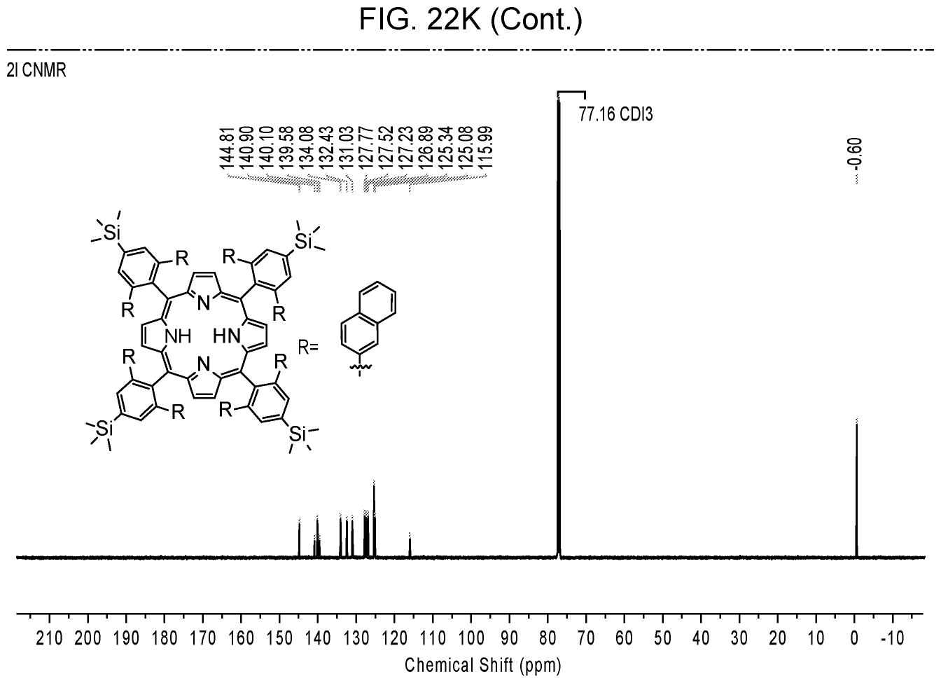

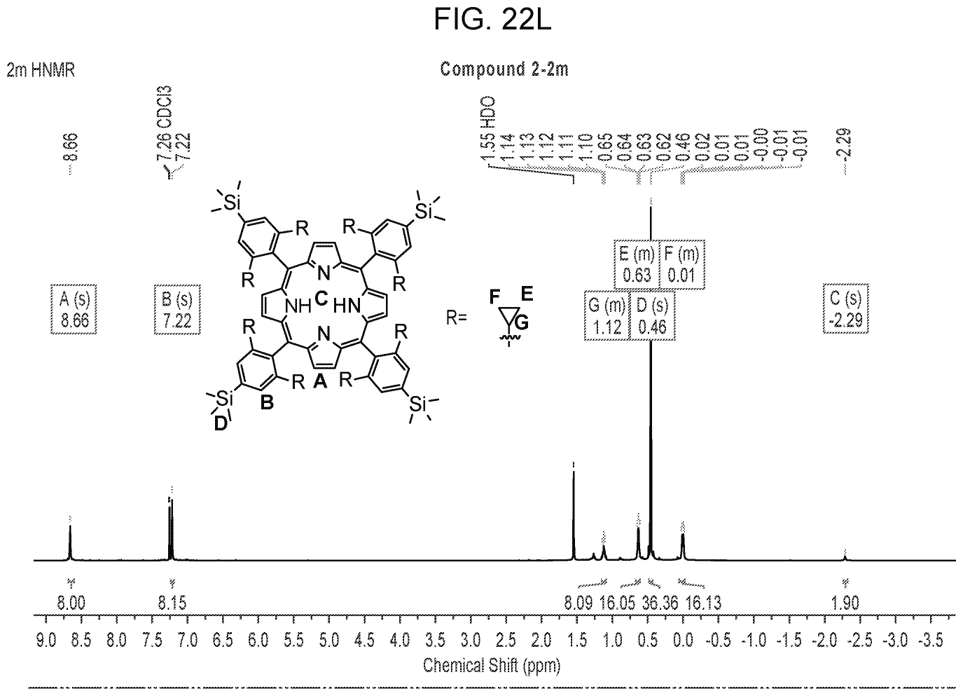

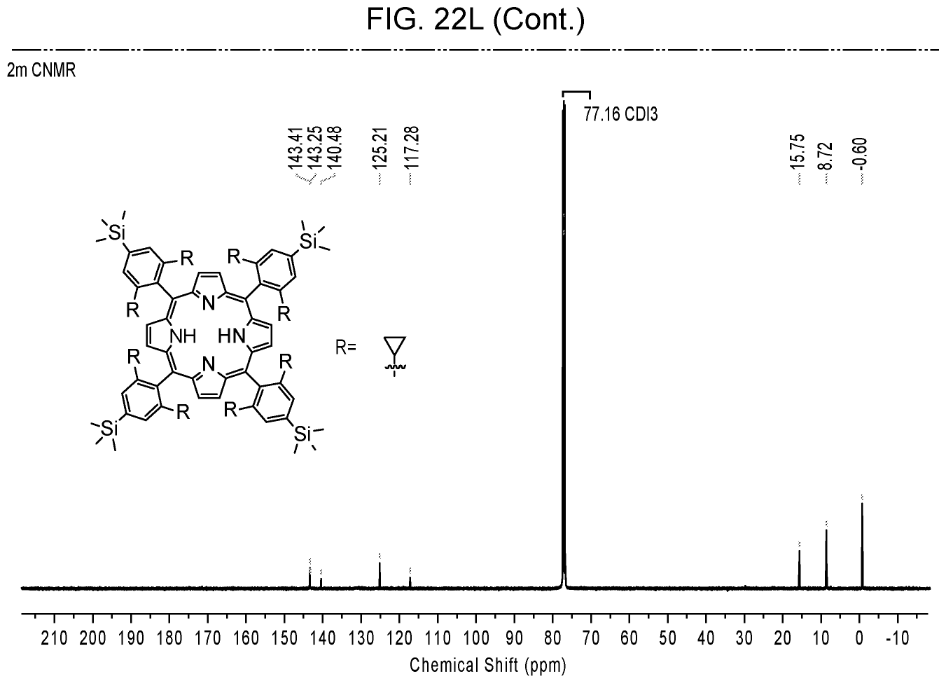

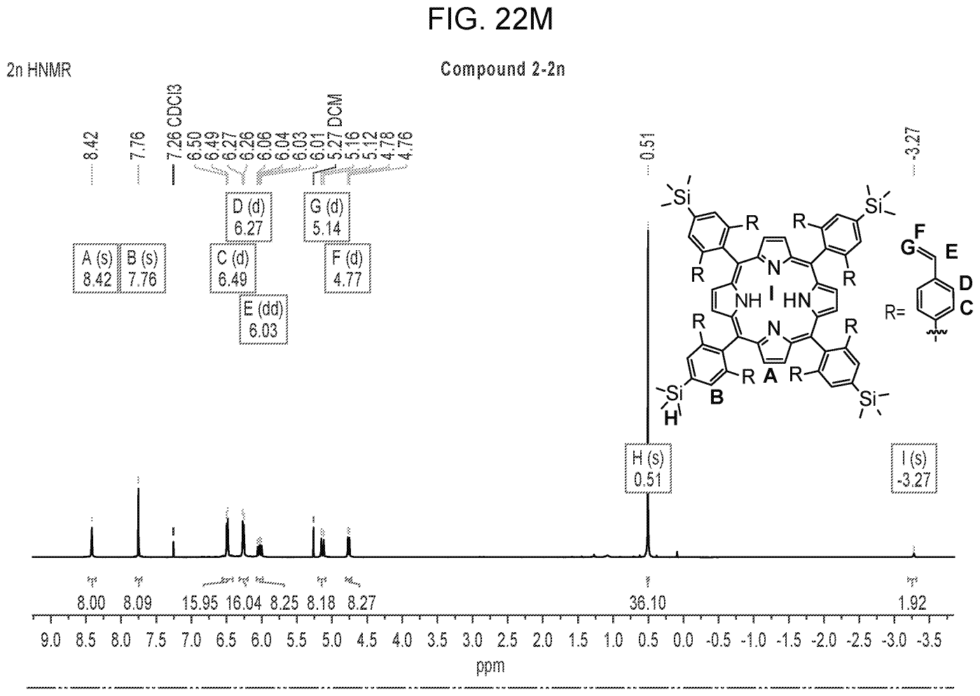

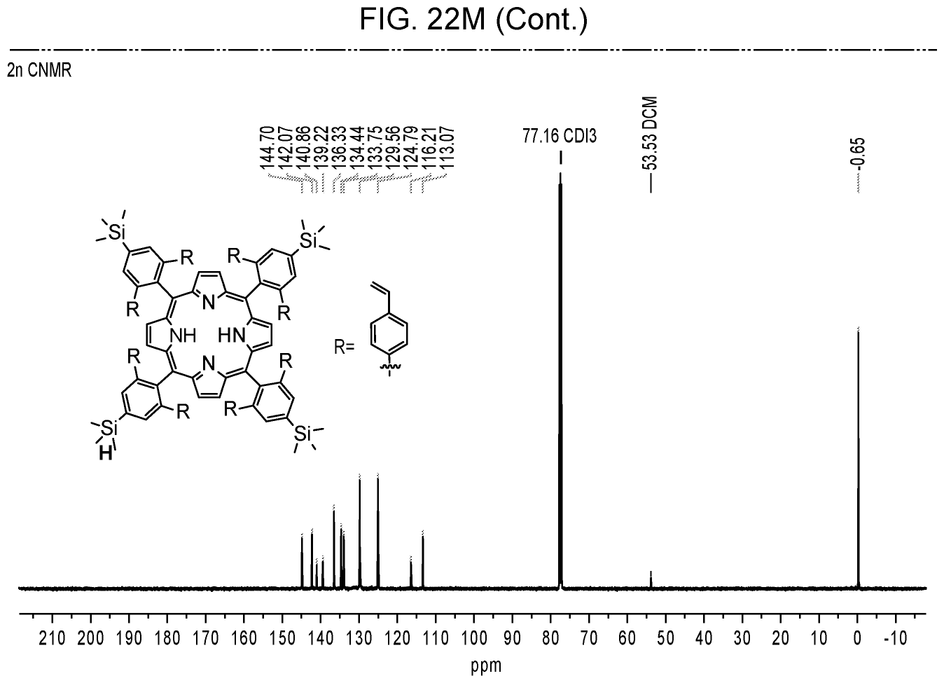

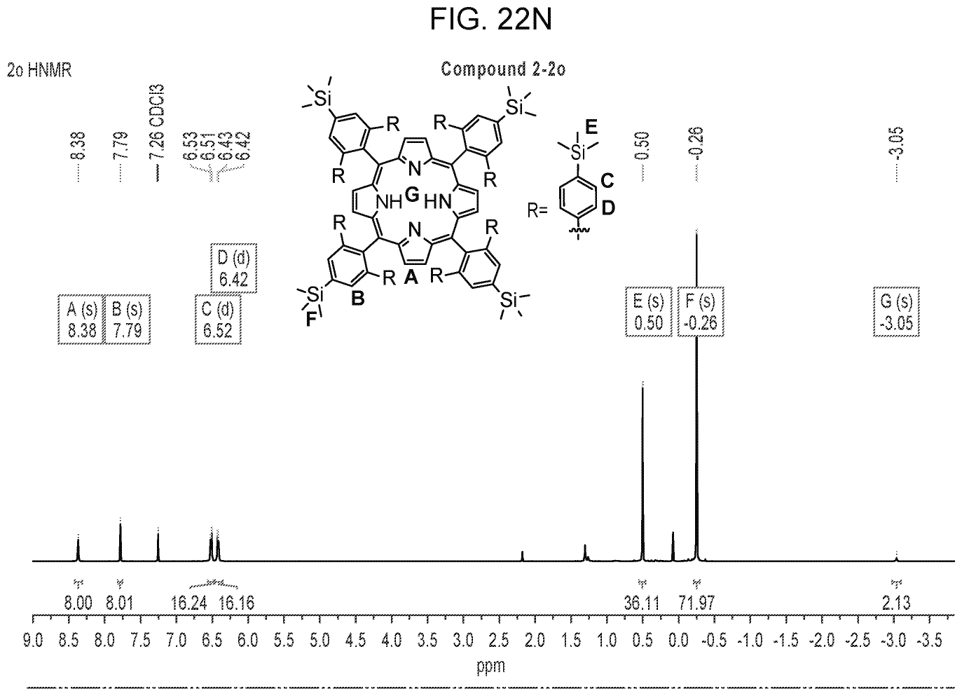

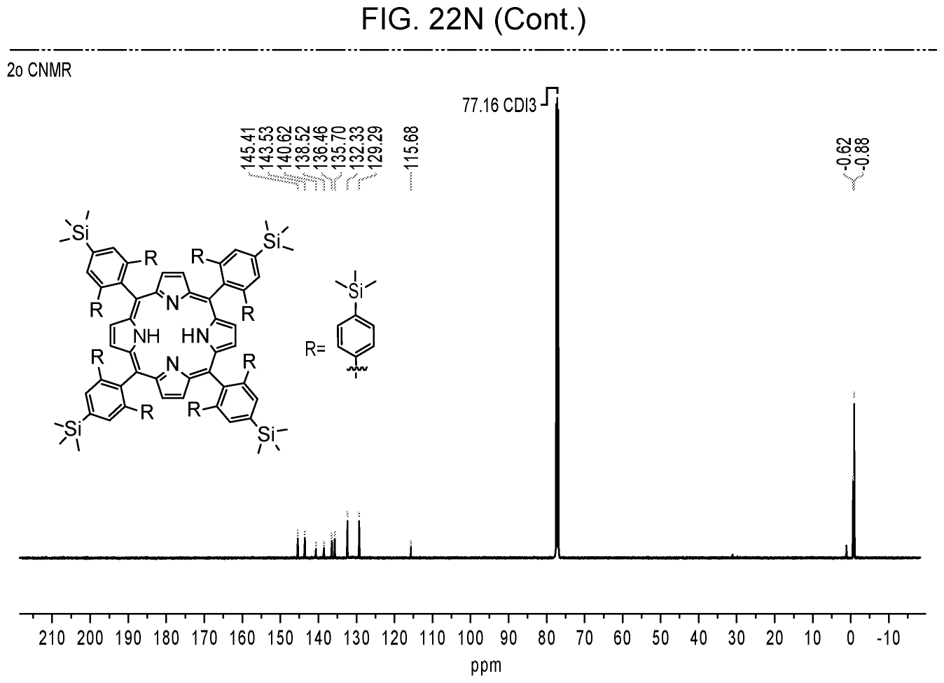

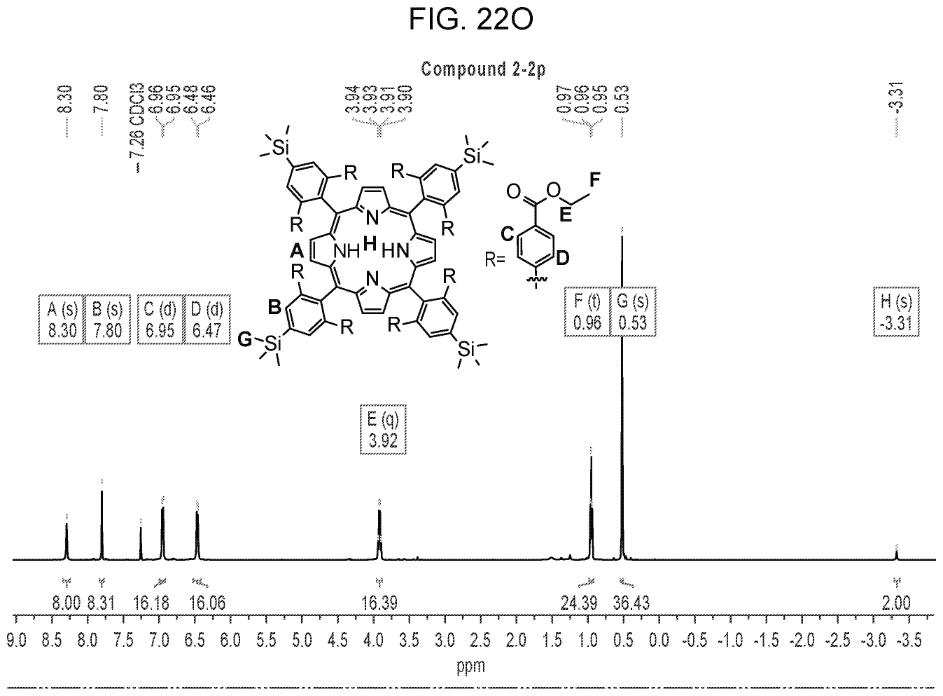

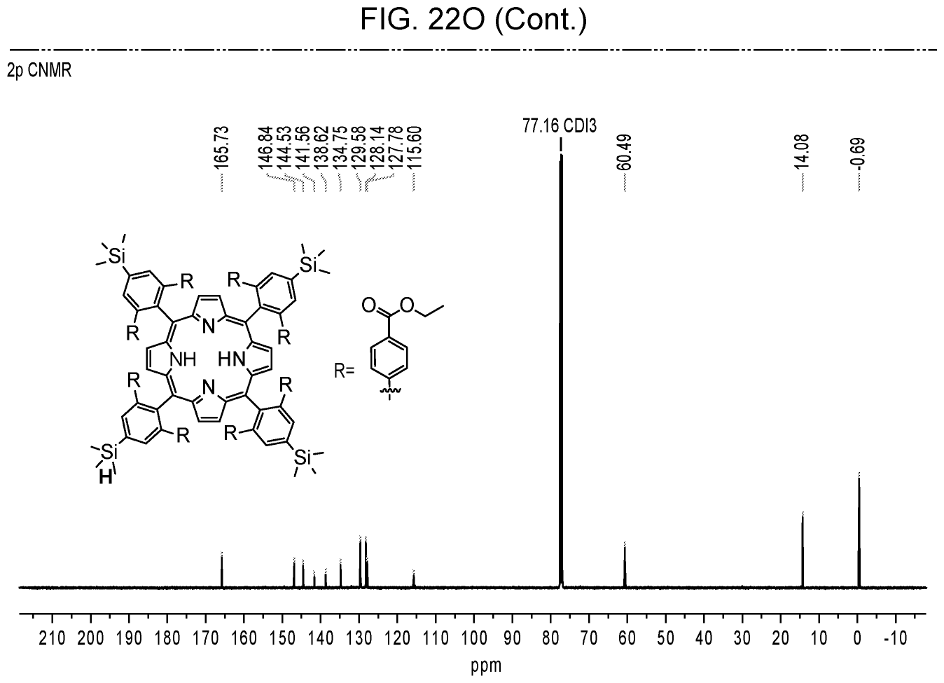

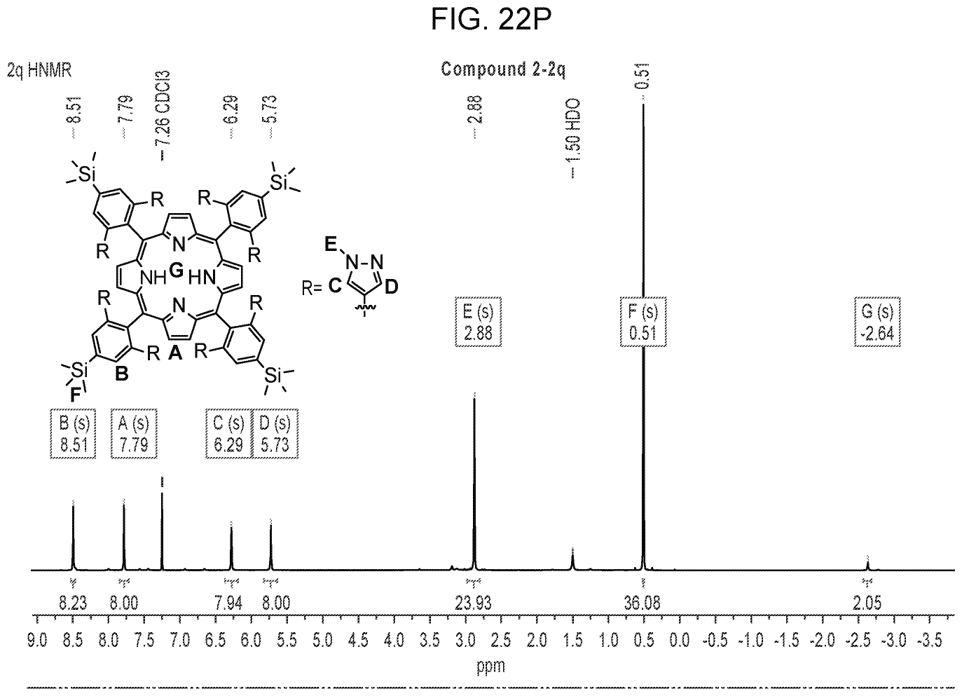

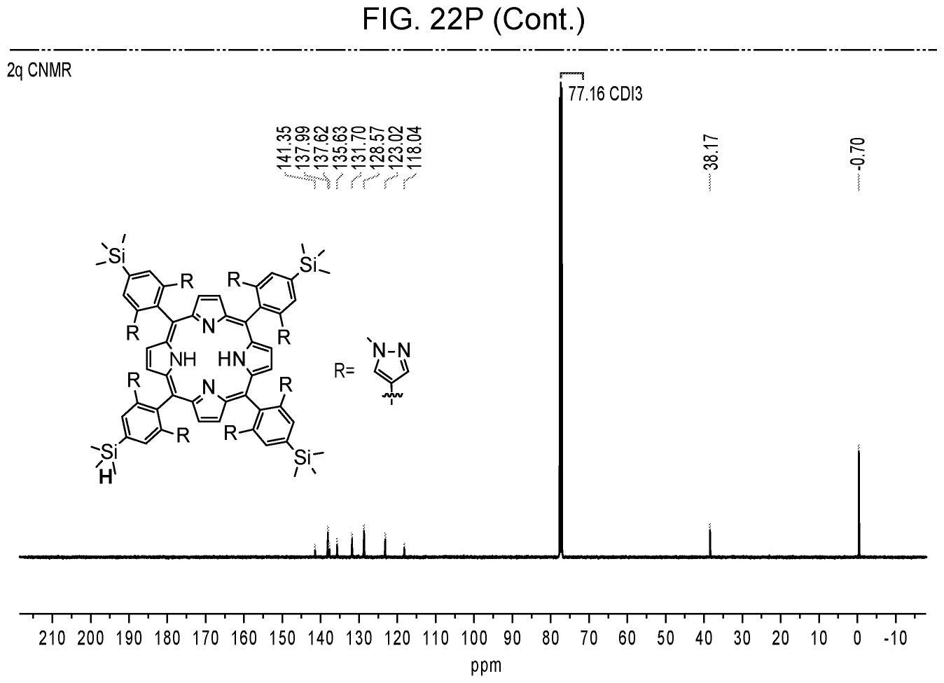

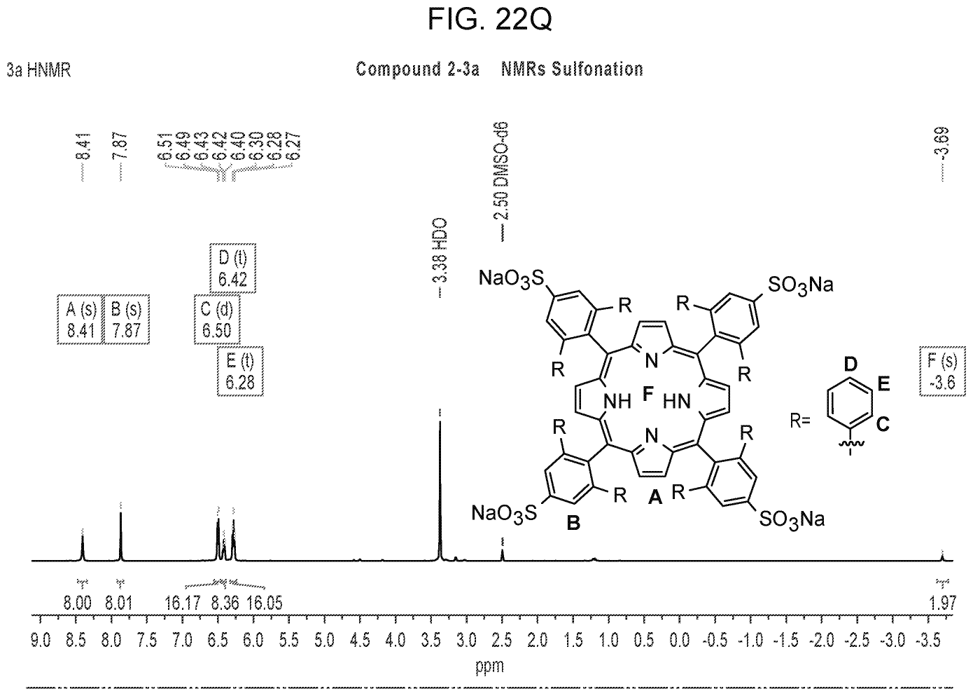

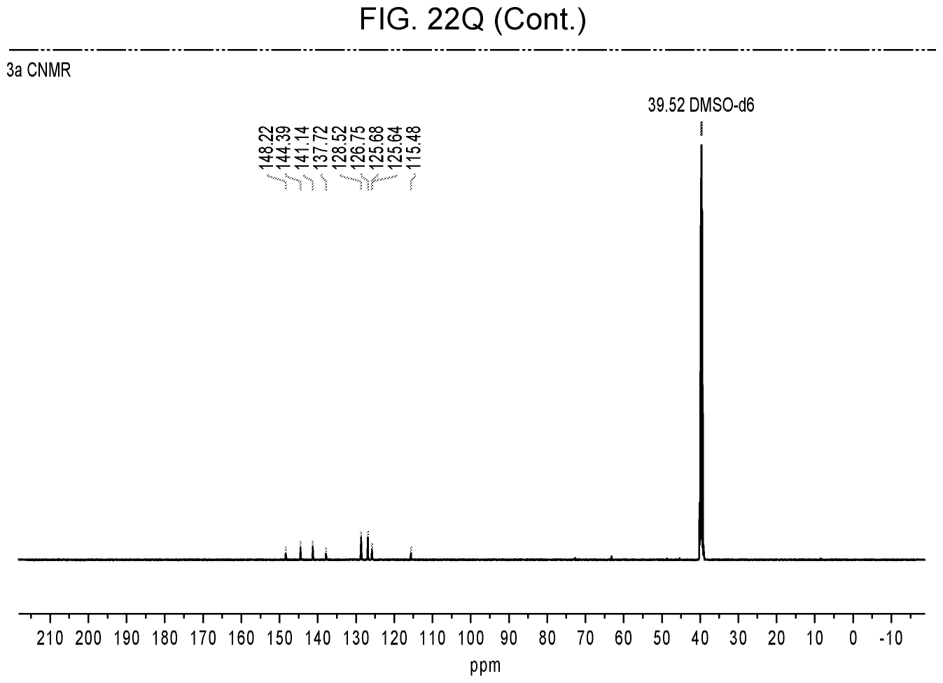

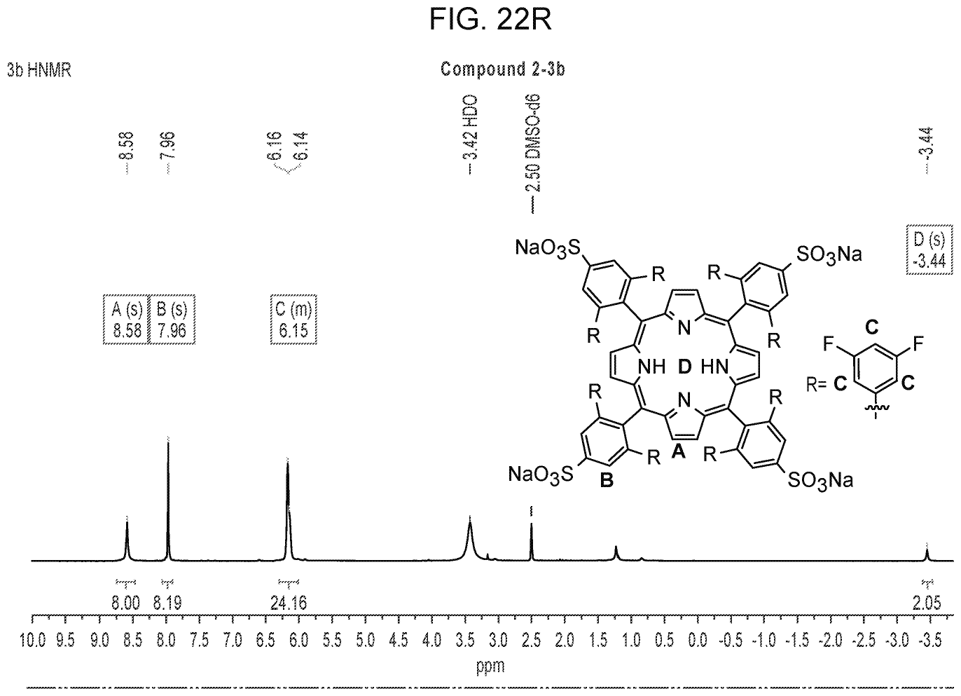

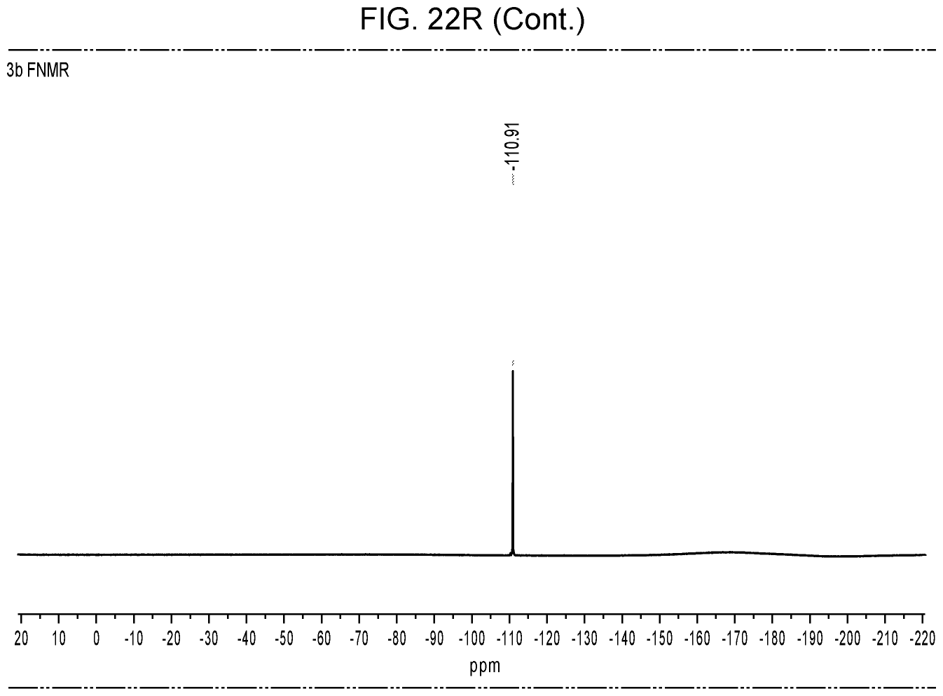

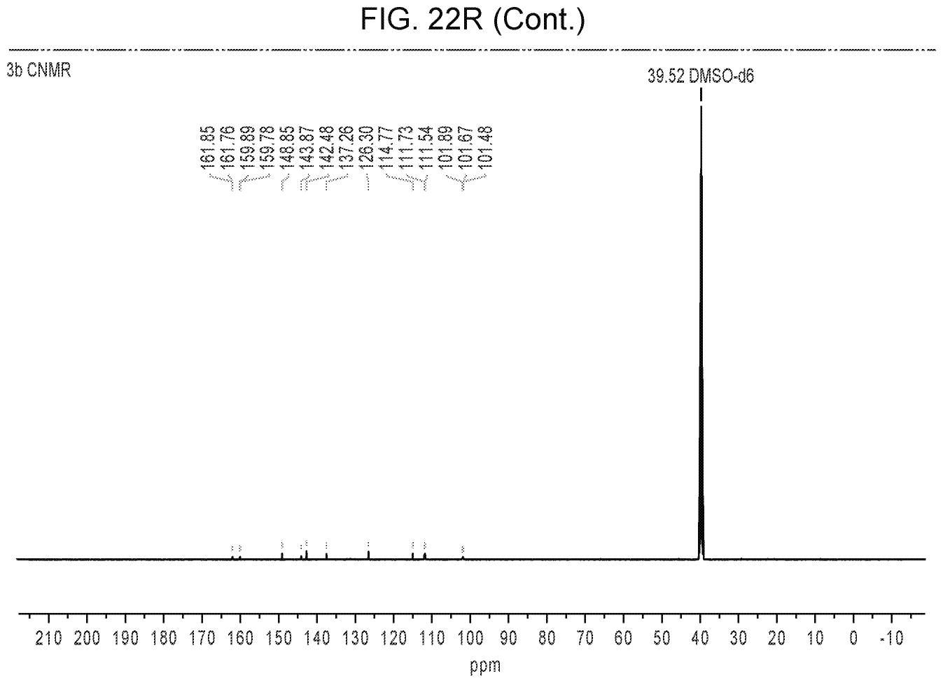

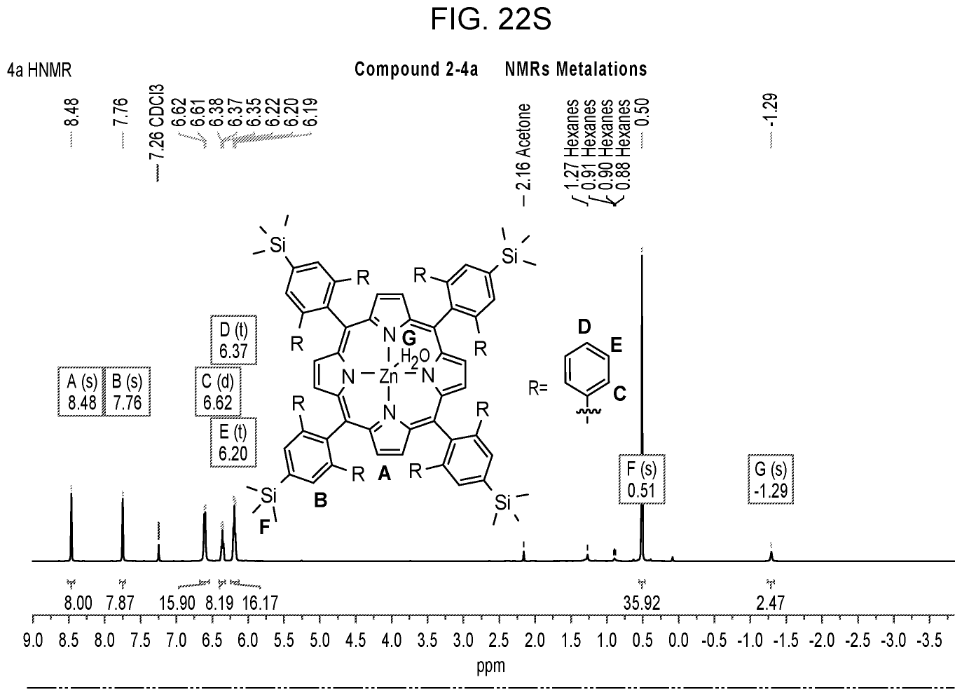

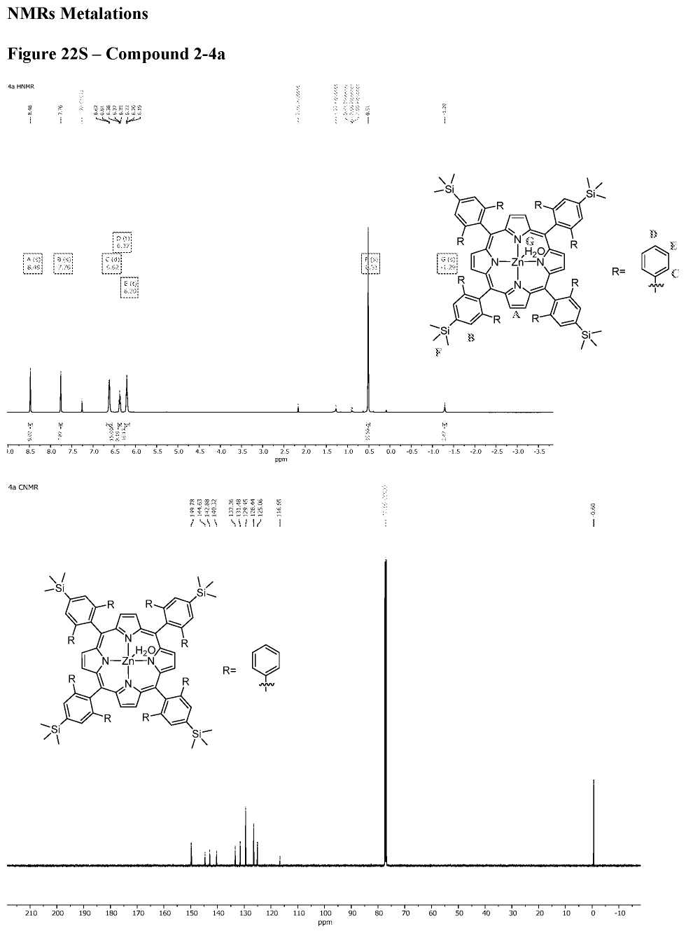

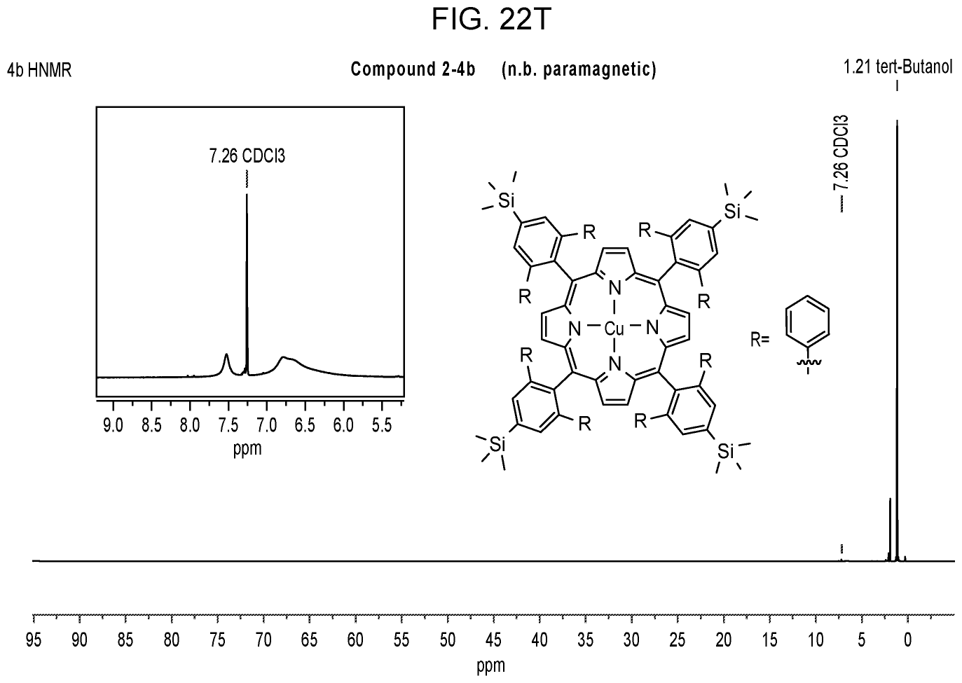

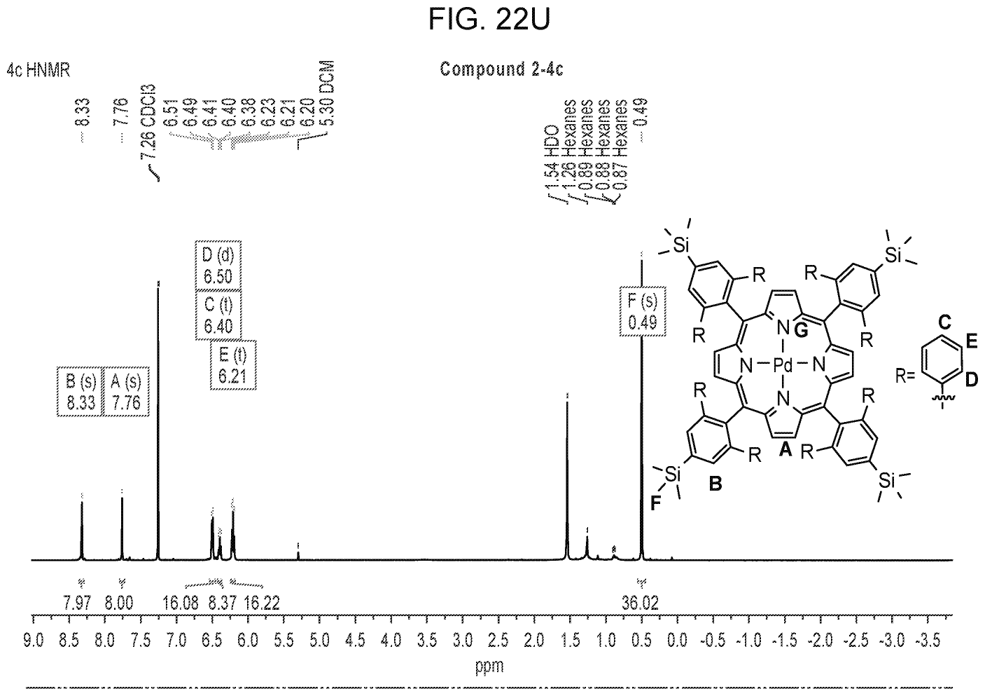

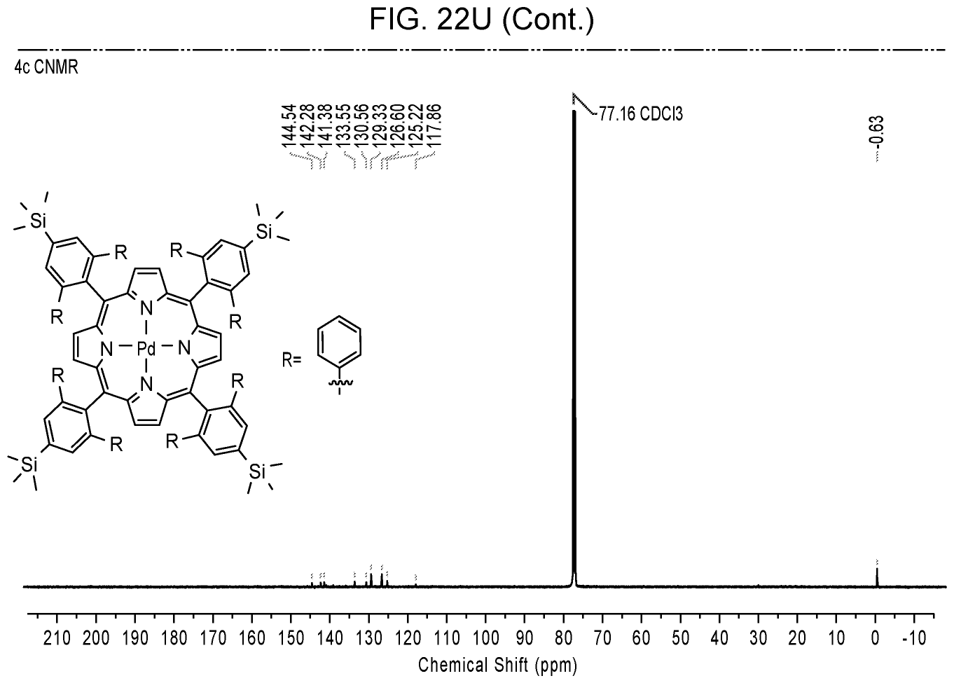

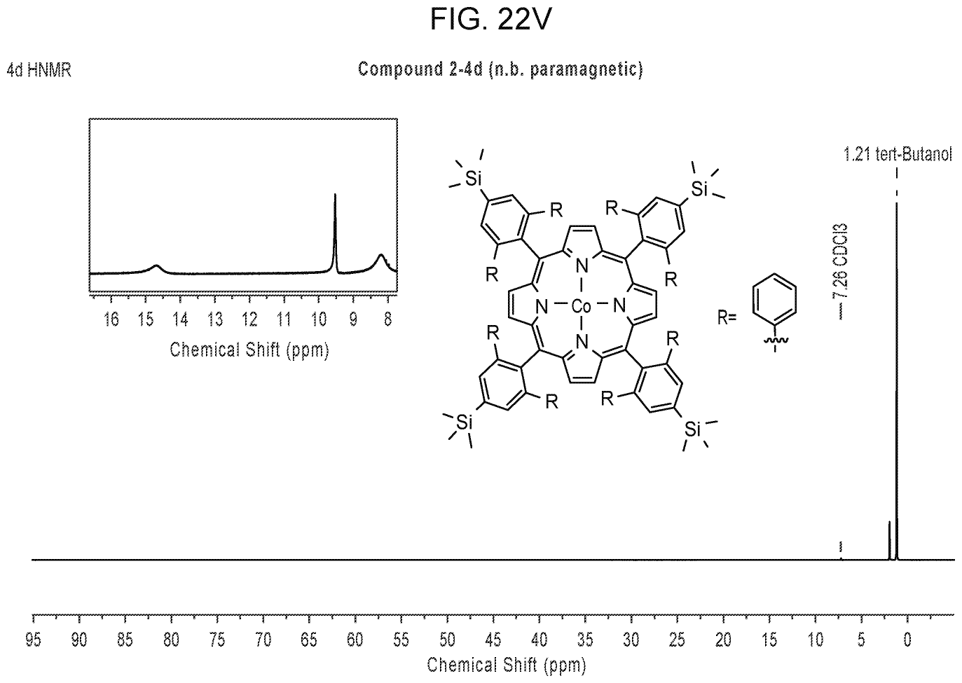

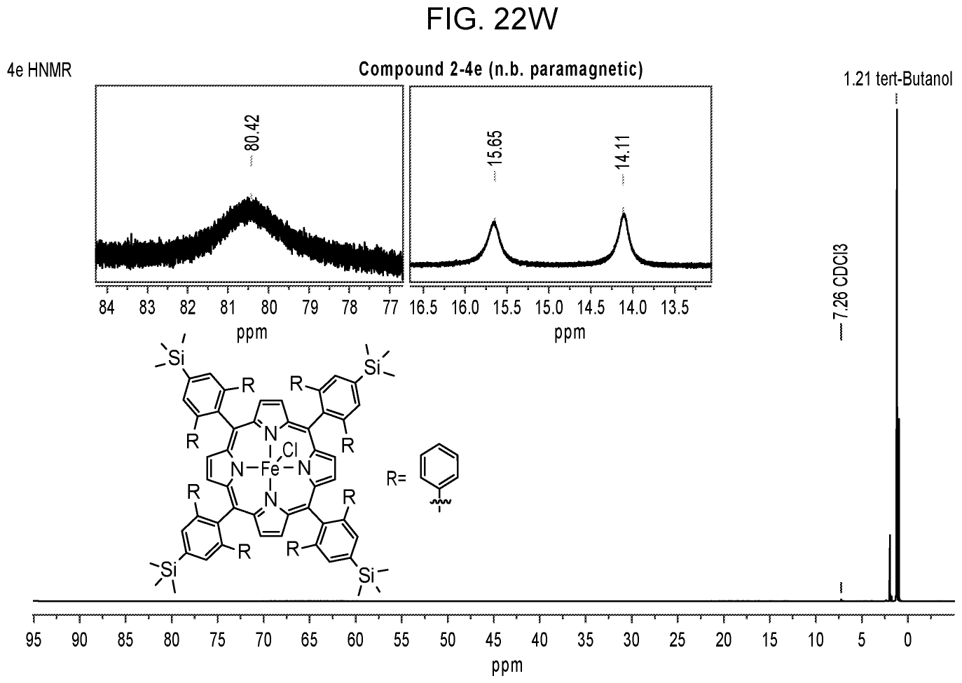

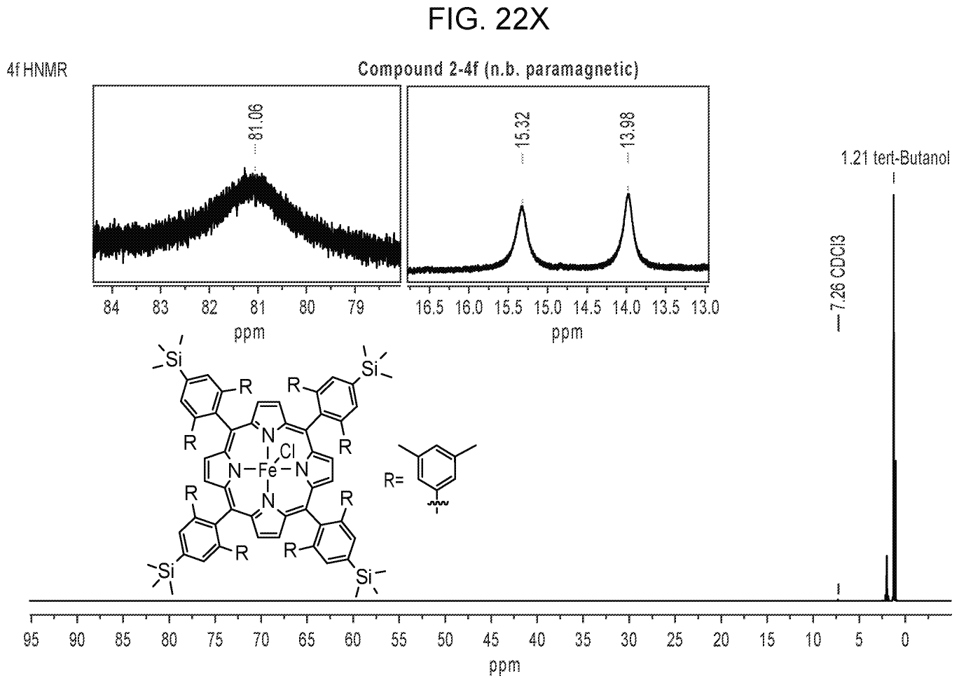

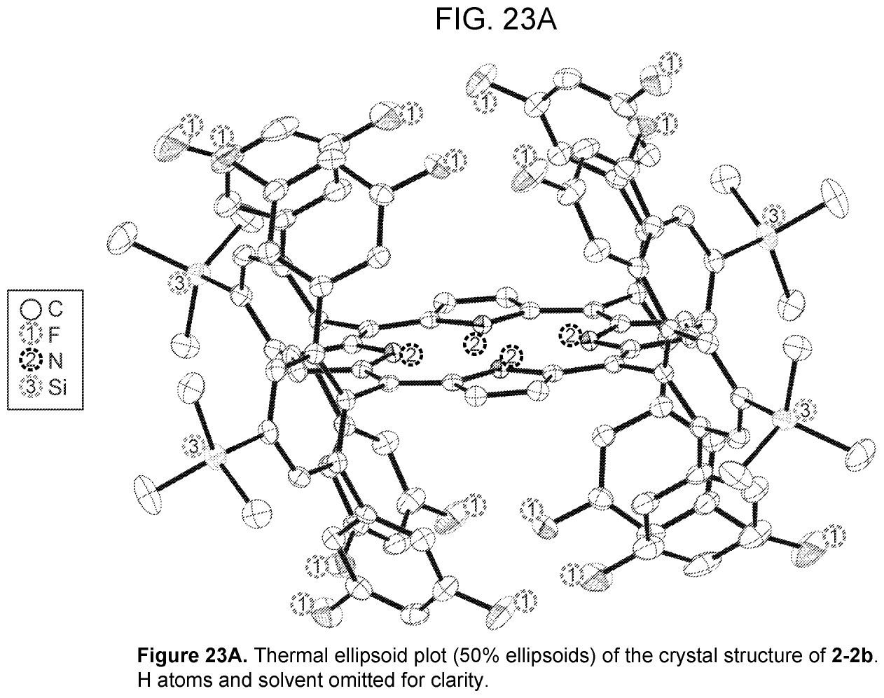

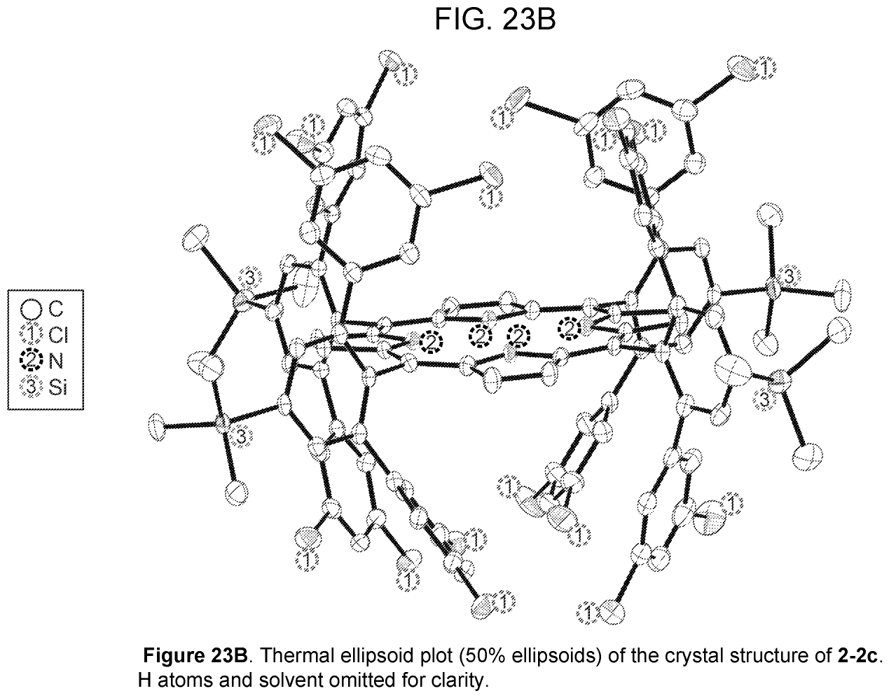

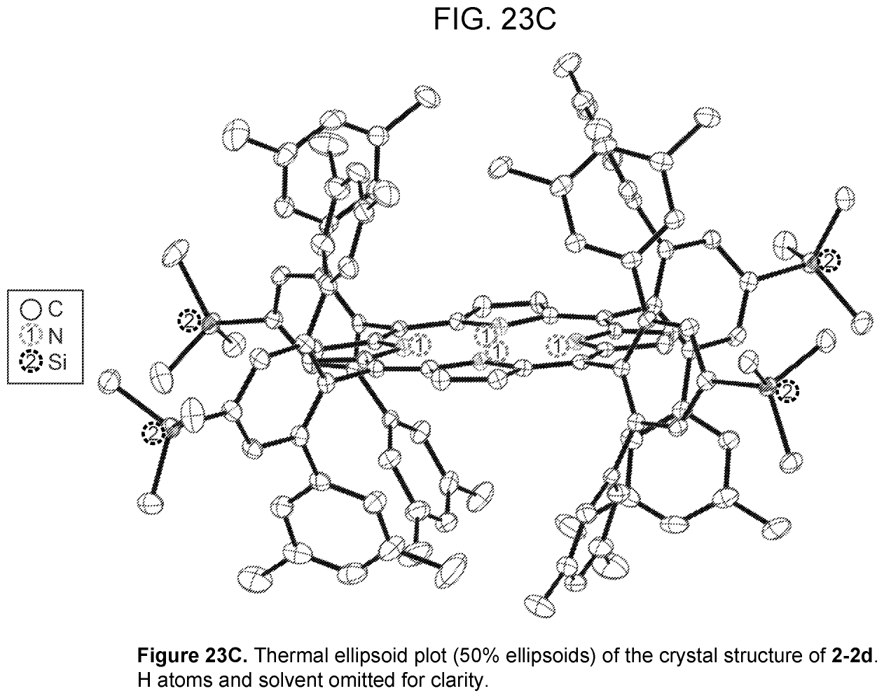

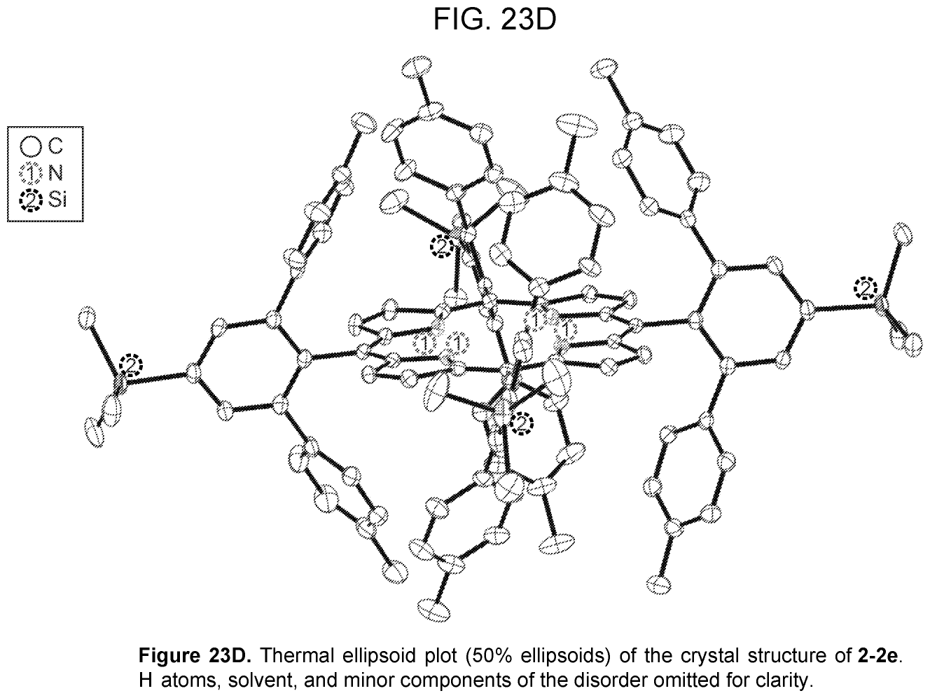

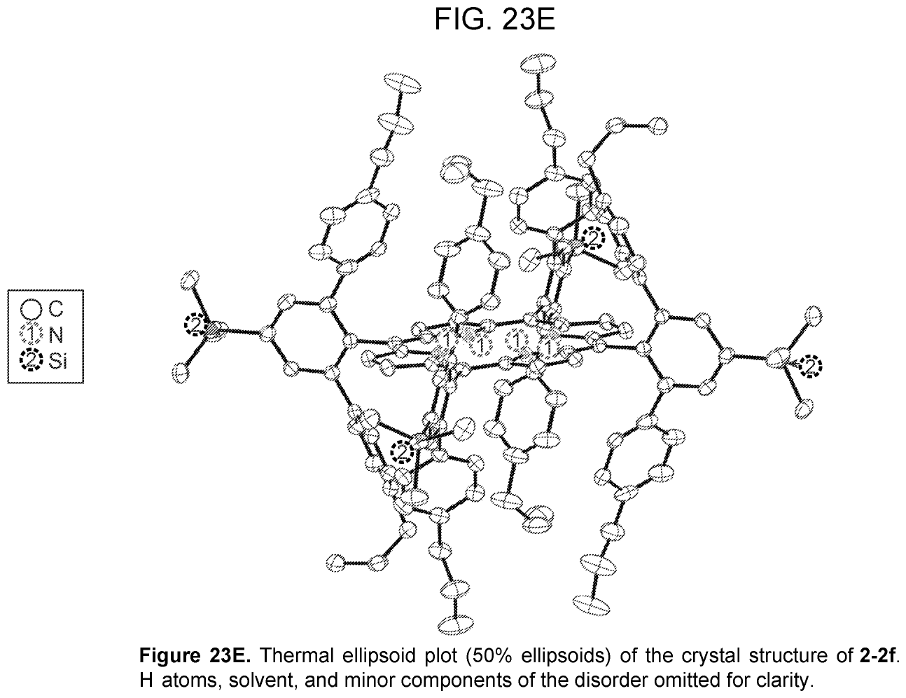

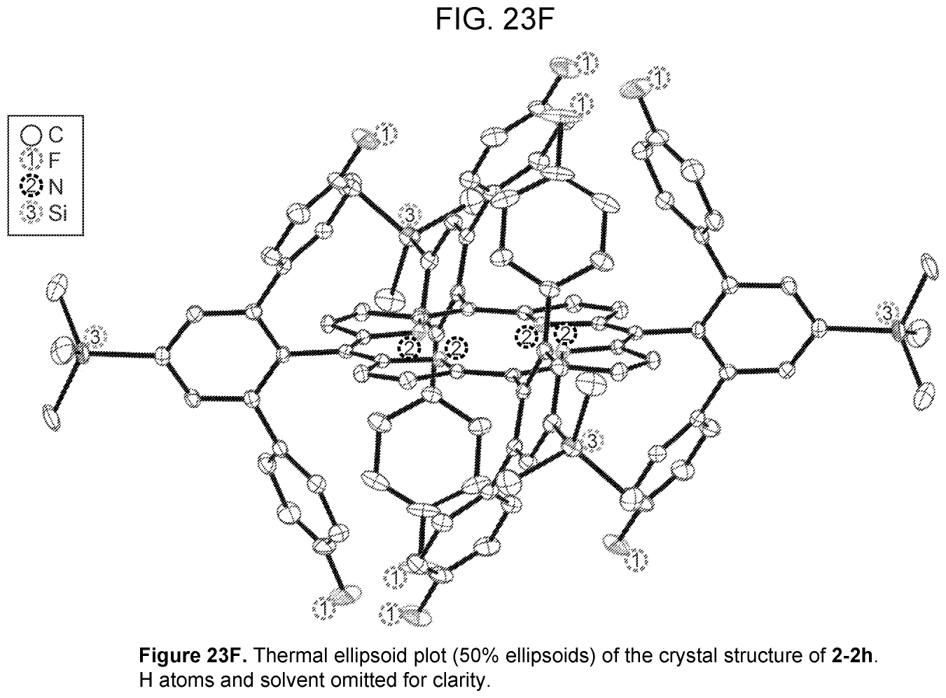

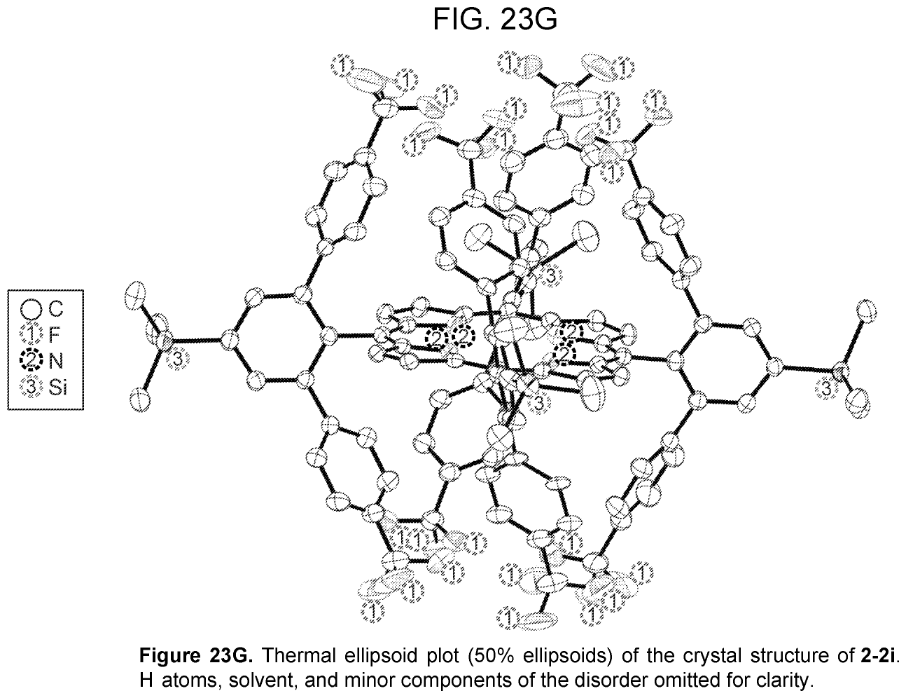

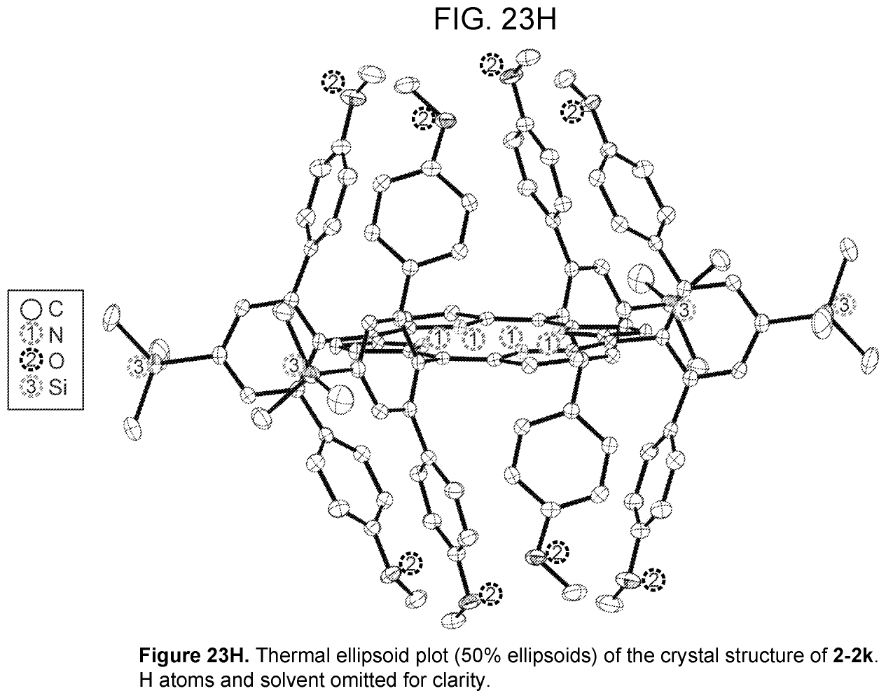

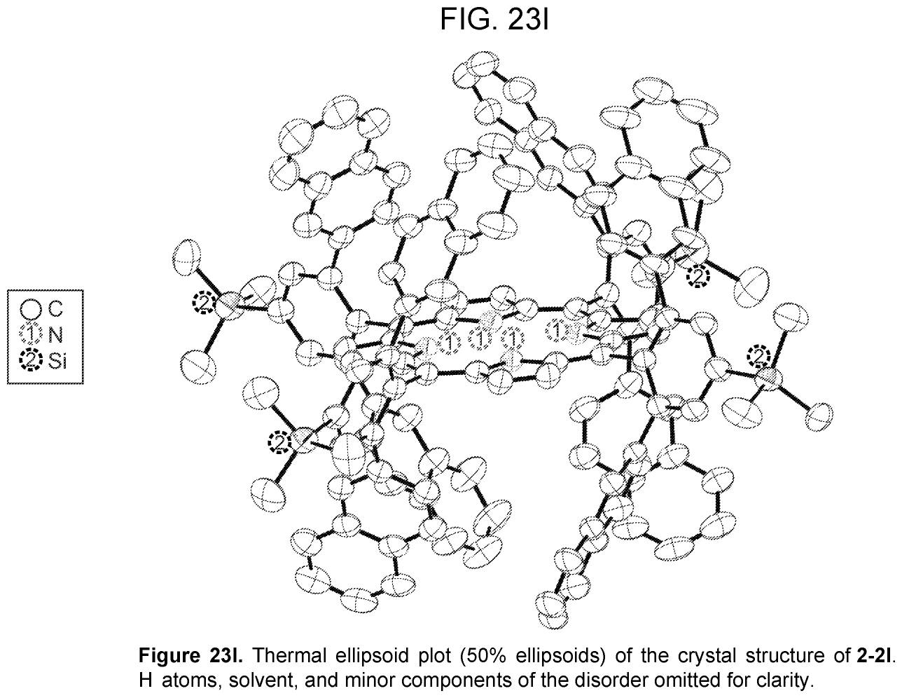

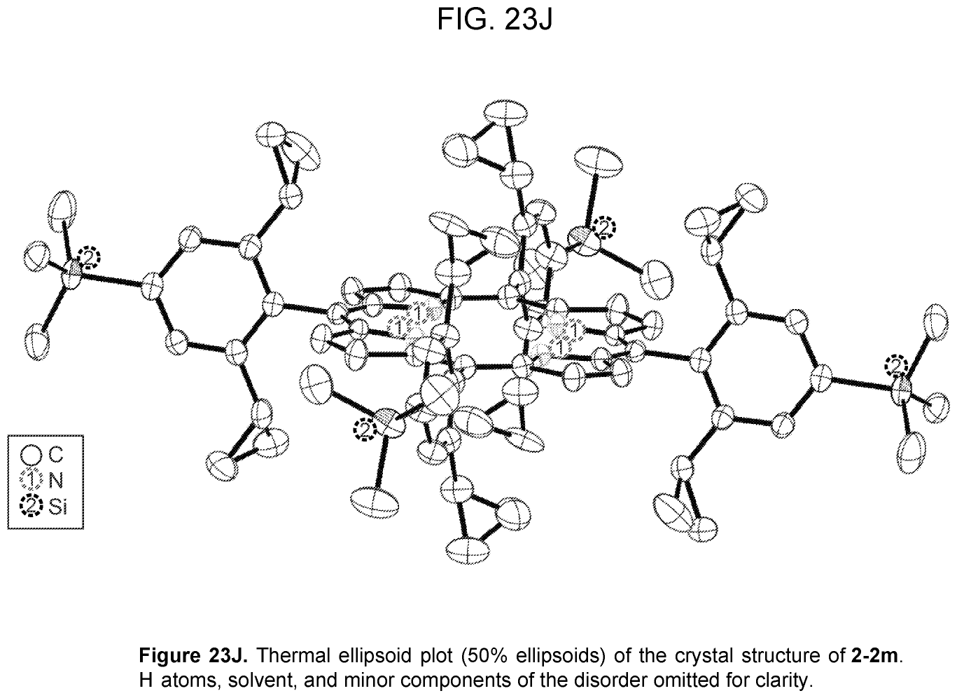

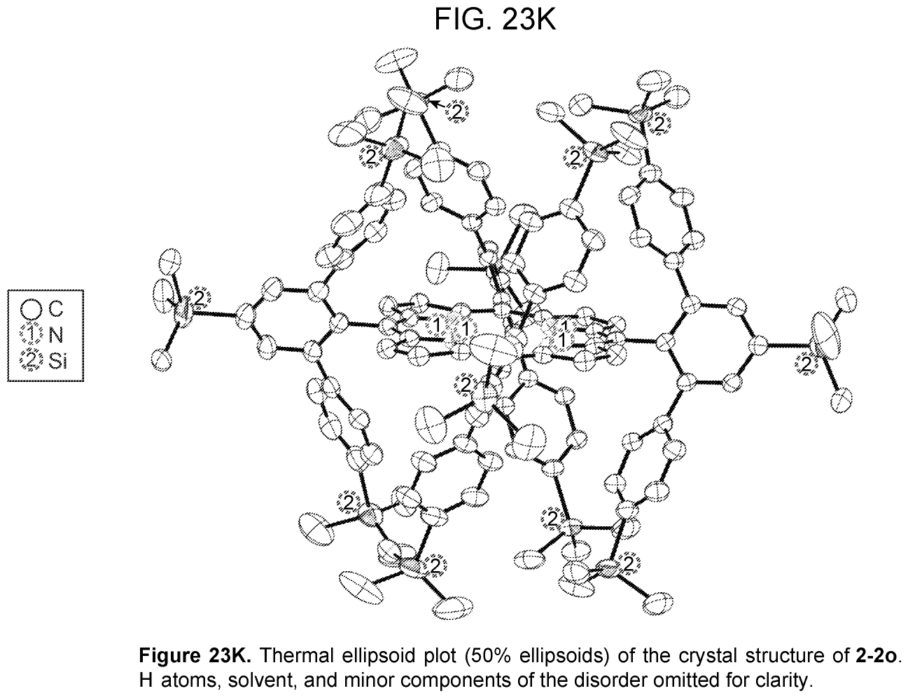

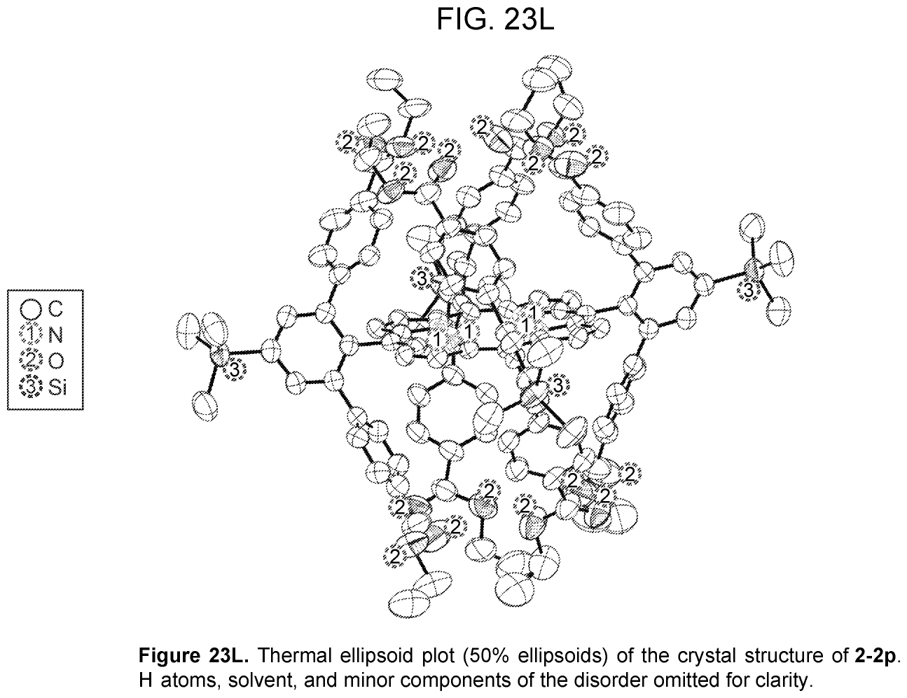

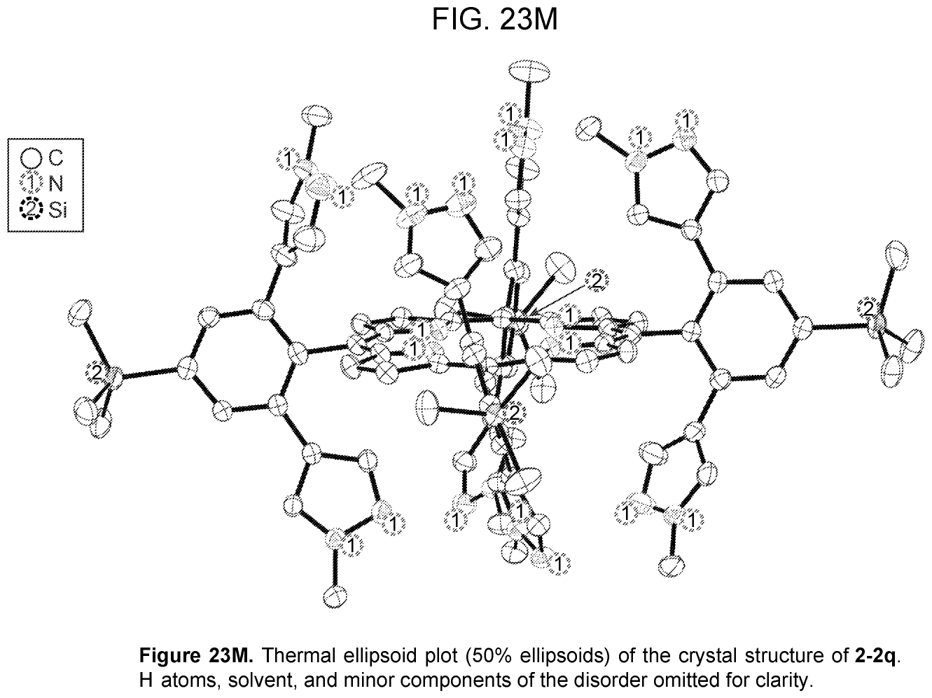

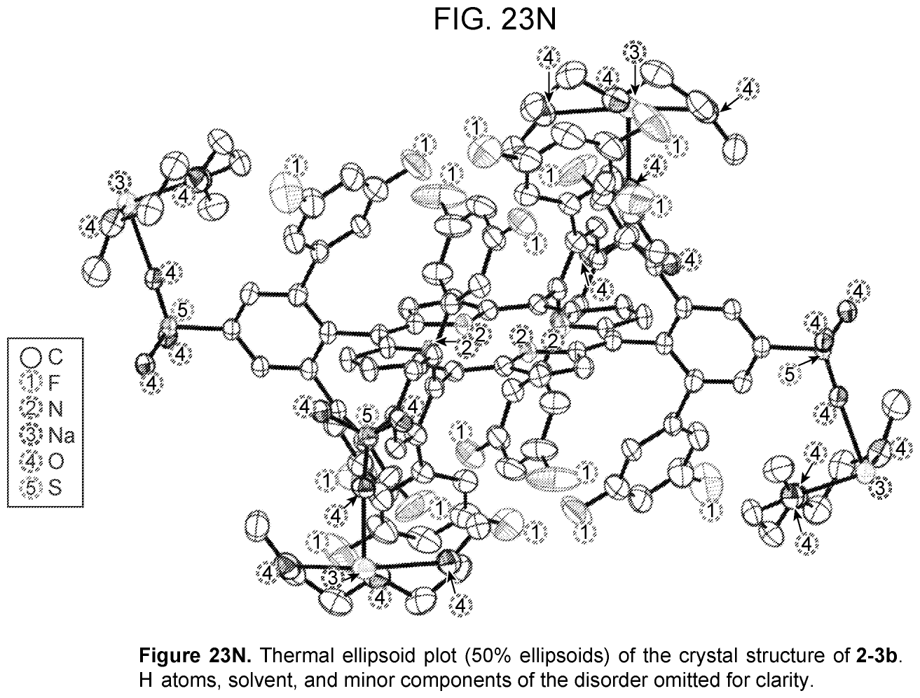

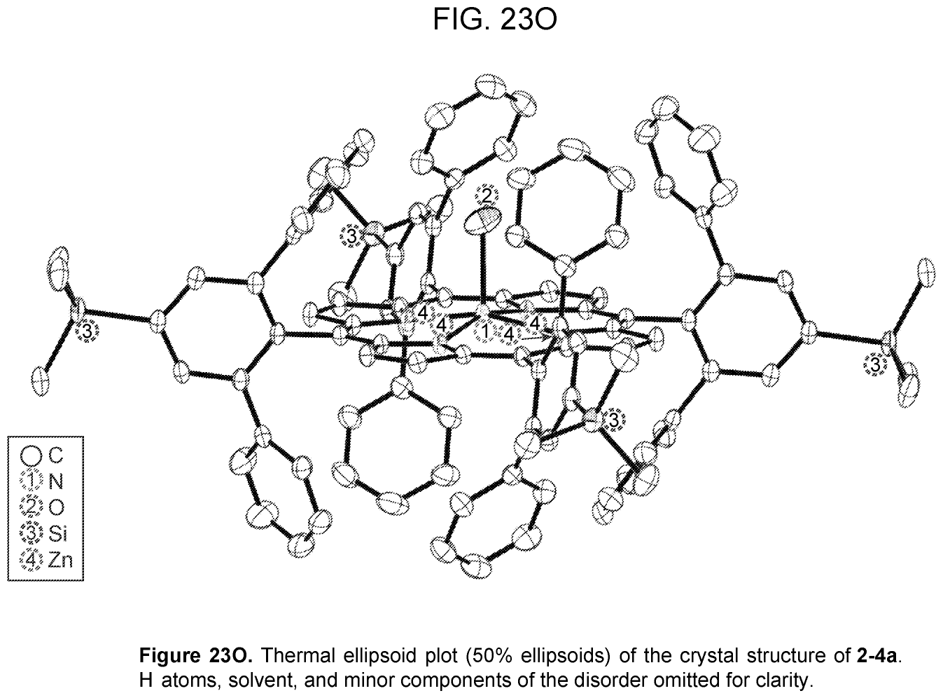

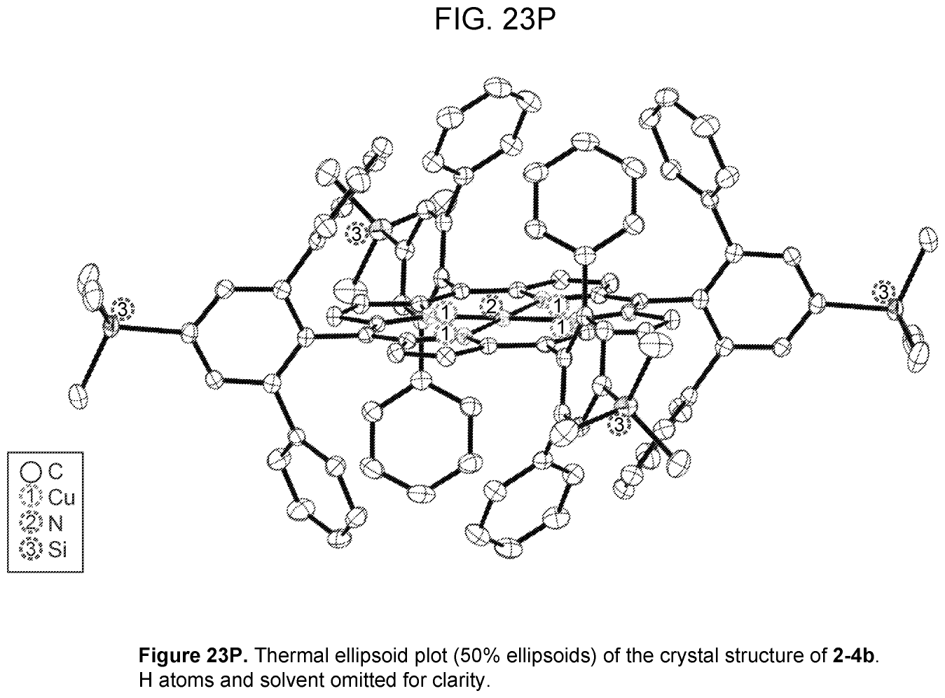

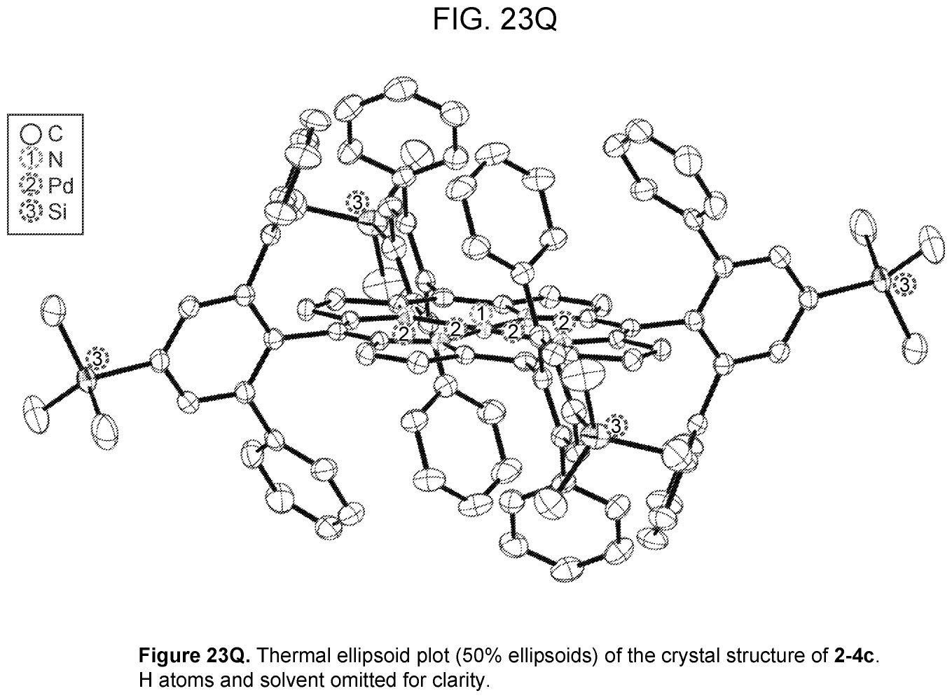

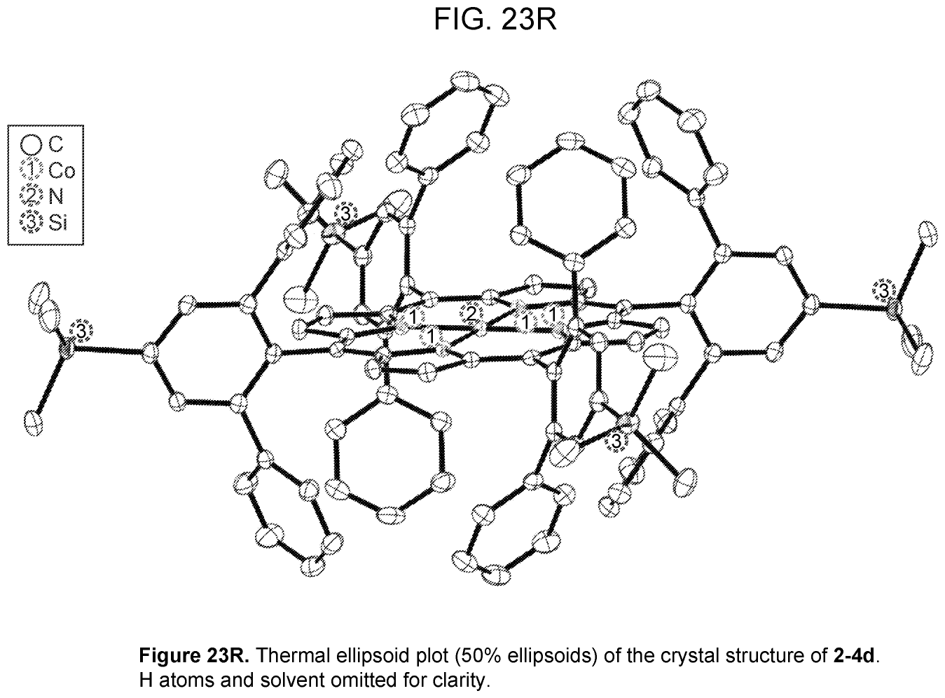

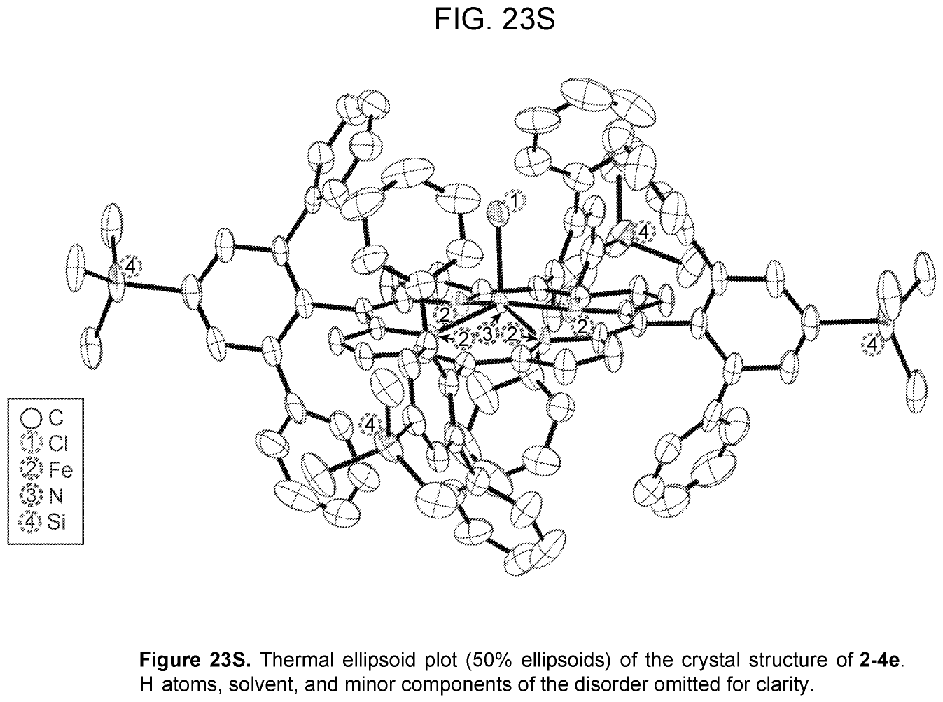

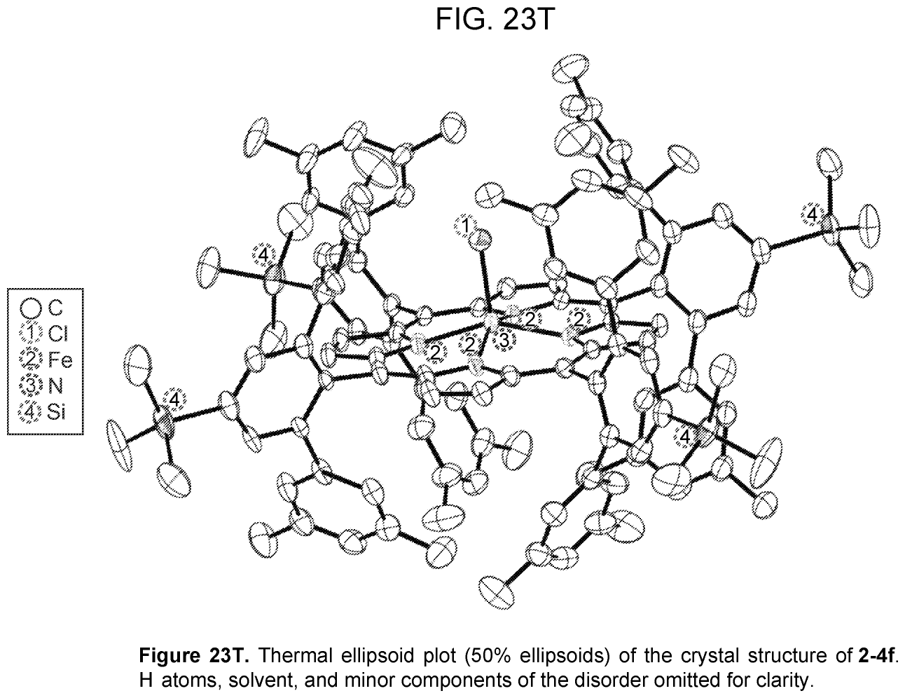

FIGS. 22A-22X depict the NMR spectra for Compound 2-2b (FIG. 22A), Compound 2-2c (FIG. 22B), Compound 2-2d (FIG. 22C), Compound 2-2e (FIG. 22D), Compound 2-2f (FIG. 22E), Compound 2-2g (FIG. 22F), Compound 2-2h (FIG. 22G), Compound 2-2i (FIG. 22H), Compound 2-2j (FIG. 22I), Compound 2-2k (FIG. 22J), Compound 2-21 (FIG. 22K), Compound 2-2m (FIG. 22L), Compound 2-2n (FIG. 22M), Compound 2-20 (FIG. 22N), Compound 2-2p (FIG. 22I), Compound 2-2q (FIG. 22P), Compound 2-3a (FIG. 22Q), Compound 2-3b (FIG. 22R), Compound 2-4a (FIG. 22S), Compound 2-4b (FIG. 22T), Compound 2-4c (FIG. 22U), Compound 2-4d (FIG. 22V), Compound 2-4e (FIG. 22W), Compound 2-4f (FIG. 22X). FIGS. 23A-23T depict the thermal ellipsoid plot of the crystal structures for Compound 2-2b (FIG. 23A), Compound 2-2c (FIG. 23B), Compound 2-2d (FIG. 23C), Compound 2-2e (FIG. 23D), Compound 2-2f (FIG. 23E), Compound 2-2h (FIG. 23F), Compound 2-2i (FIG. 23G), Compound 2-2k (FIG. 23H), Compound 2-21 (FIG. 23I), Compound 2-2m (FIG. 23J), Compound 2-20 (FIG. 23K), Compound 2-2p (FIG. 23L), Compound 2-2q (FIG. 23M), Compound 2-3b (FIG. 23N), Compound 2-4a (FIG. 23I), Compound 2-4b (FIG. 23P), Compound 2-4c (FIG. 23Q), Compound 2-4d (FIG. 23R), Compound 2-4e (FIG. 23S) and Compound 2-4f (FIG. 23T).

### Results And Discussion

Reaction Optimization. In Example 1, the synthesis of Compound 2-2a was accessed via Pd-catalyzed cross-coupling of 1 and PhB(OH)₂. This Suzuki-Miyaura reaction was performed over 16 h in 20:1 1,4-dioxane/water using a three-fold excess of PhB(OH)₂ (per Ar-Br bond), Cs₂CO₃ as a base, and 12.5 mol % (dppf) PdCl₂ (per Ar-Br bond). These conditions were used as the starting point for optimization as described here for the coupling of 1 with arylboronic acids. Here, optimizing the yield while minimizing the amount of catalyst used; the initial 12.5 mol % (per Ar-Br bond) ensured that the product was obtained in our previous work,21 but is assuredly in excess of the amount needed to achieve this goal.

The amount of Pd catalyst (Table 2-9) was iteratively decreased. The yield of the reaction remained unchanged with a catalyst loading as low as 1 mol % (per Ar-Br bond). The loading could be further reduced to 0.5 mol % but a longer reaction time of 20 h was needed to ensure that the reaction proceeded to completion. A loading of 0.1 mol % resulted in negligible formation of product, even at the longer reaction time. Subsequent steps in the optimization were performed using 1 mol % catalyst.

TABLE 2-9
Catalyst Loading for Coupling ^{a}

| Catalyst | mol % cat | Yield | Time (h) |
|---|---|---|---|
| (dppf)PdCl₂ | 12.5 | 76% | 16 |
| (dppf)PdCl₂ | 5 | 77% | 16 |
| (dppf)PdCl₂ | 1 | 73% | 16 |
| (dppf)PdCl₂ | 0.5 | 60% | 16 |
| (dppf)PdCl₂ | 0.5 | 72% | 20 |
| (dppf)PdCl₂ | 0.1 | 0% | 20 |
| None | 0 | 0% | 20 |

^{a} mol % of catalyst, equiv PhB(OH)₂, and equiv base are provided with respect to Ar—Br bonds

A panel of Pd catalysts was screened with a focus on complexes that facilitate coupling reactions, in particular those used to catalyze the coupling of sterically hindered substituents

(Table 2-10). Pd(PPh₃)₄ was observed to perform comparably to (dppf) PdCl₂. Pd catalysts with bidentate ligands, such as Xantphos and rac-BINAP, afforded greater yields than catalysts with the monodentate ligands SPhos and DavePhos. The precatalyst alone afforded no product. Under our initial set of reaction conditions, (dppf)PdCl₂ and Pd(PPh₃)₄ were the only catalysts tested that allowed the reaction to reach completion; all others gave mixtures of the desired product and various partially functionalized intermediates with fewer than eight of the aryl bromides having reacted. These partially substituted intermediates can be separated from the desired product by silica gel column chromatography, but even with extensive optimization, resolution of the intermediates and product was poor. To quantitatively evaluate the different catalysts (and other reaction conditions, vide infra), all partially substituted intermediates along with the product were collected and determined the fraction of that porphyrinic material corresponding to the desired product using NMR spectroscopy. Although the aromatic ¹H NMR resonances of these species overlap, the N—H signals, characteristically shifted upfield (δ<−2 ppm) because of their position within the center of the strong diamagnetic ring current of the porphyrin macrocycle, are well separated. Indeed, the reactions could be monitored readily by observing progressive growth and disappearance of the N—H signal of each intermediate. The yields reported below were calculated by multiplying the mass of the total isolated porphyrinic material by the quotient of the integral of the N—H resonance of the desired product and the integral of all N—H resonances in the isolated material. Although Pd(PPh₃) s afforded a reaction yield comparable to that of (dppf)PdCl₂, we chose to continue our optimization with (dppf)PdCl due to its relatively low cost, case of use, and benchtop stability.

TABLE 2-10
Coupling with PhB(OH)₂ ^{a}

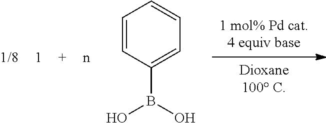

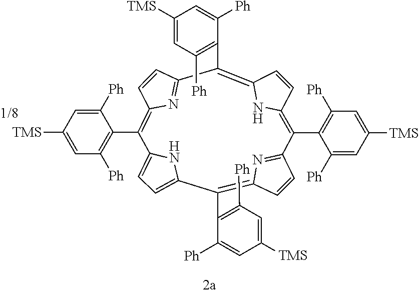 [SMILES: C[Si](C)(C)c1cc(-c2ccccc2)c(-c2c3nc(c(-c4c(-c5ccccc5)cc([Si](C)(C)C)cc4-c4ccccc4)c4ccc([nH]4)c(-c4c(-c5ccccc5)cc([Si](C)(C)C)cc4-c4ccccc4)c4nc(c(-c5c(-c6ccccc6)cc([Si](C)(C)C)cc5-c5ccccc5)c5ccc2[nH]5)C=C4)C=C3)c(-c2ccccc2)c1]

| | Equiv | | | |
|---|---|---|---|---|
| Catalyst | PhB(OH)₂ Base | | Solvent | Yield |
| Pd(PPh₃)₄ | 3 | Cs₂CO₃ | Dioxane | 79% |
| Xantphos^{b} | 3 | Cs₂CO₃ | Dioxane | 57% |
| rac-BINAP^{b} | 3 | Cs₂CO₃ | Dioxane | 70% |
| SPhos^{b} | 3 | Cs₂CO₃ | Dioxane | 8% |
| DavePhos^{b} | 3 | Cs₂CO₃ | Dioxane | 3% |
| Pd₂(DBA)₃^{c} | 3 | Cs₂CO₃ | Dioxane | 0% |
| (dppf)PdCl₂ | 3 | KOH | Dioxane | 0% |
| (dppf)PdCl₂ | 3 | KOAc | Dioxane | 51% |
| (dppf)PdCl₂ | 3 | K₃PO₄ | Dioxane | 57% |
| (dppf)PdCl₂ | 3 | K₂CO₃ | Dioxane | 36% |
| (dppf)PdCl₂ | 3 | Cs₂CO₃ | DMF | 0% |
| (dppf)PdCl₂ | 3 | Cs₂CO₃ | Toluene | 89% |
| (dppf)PdCl₂ | 4 | Cs₂CO₃ | Toluene | 93% |
| (dppf)PdCl₂ | 2 | Cs₂CO₃ | Toluene | 6% |
| (dppf)PdCl₂ | 3 | Cs₂CO₃^{d} | Toluene | 77% |

^{a} mol % of catalyst, equiv PhB(OH)₂, and equiv base are provided with respect to Ar—Br bonds.
^{b}1 mol % Pd₂(DBA)₃ (per Pd atom) and 2 mol % of specified ligand were precomplexed prior to the start of the reaction.
^{c}No ligand added.
^{d}3 equiv

The use of different bases was next explored in Suzuki-Miyaura couplings. KOH, which has a significantly greater pKₐ than Cs₂CO₃, formed no product. Decreasing the pKₐ (KOAc) was detrimental to the yield and the reaction did not reach completion within 20 h. Reactions with K₃PO₄ and K₂CO₃ also failed to reach completion within 20 h and featured a consequent drop in yield

Keeping Cs₂CO₃ as the base, we next investigated the influence of changing the solvent. Solvent choices were limited due the solubility of the starting porphyrin. For example, performing the reaction in DMF afforded no product, which we attribute to the poor solubility of the starting porphyrin in this solvent, even at elevated temperatures. Switching the solvent to toluene, in which the starting porphyrin is more soluble, increased the yield to 89%. Including an additional equivalent of PhB(OH)₂ in the reaction (for a total of 4 equiv) increased the yield slightly to 93%. This value reflects an isolated yield of purified, recrystallized product. Decreasing the amount of either PhB(OH)₂ or Cs₂CO₃ was detrimental to the reaction yield.

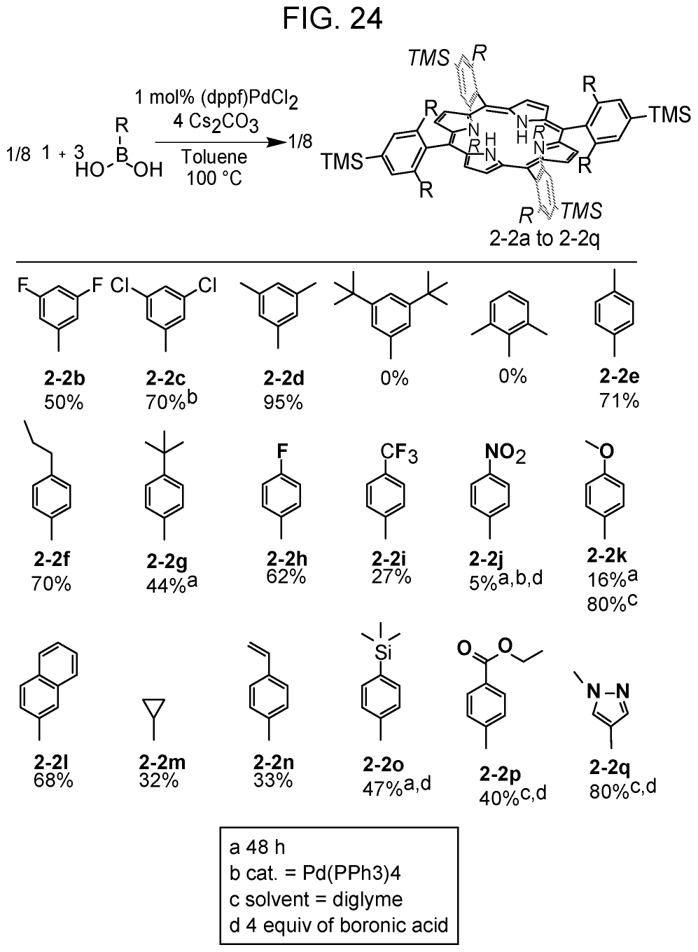

Reaction Scope. With a set of optimized reaction conditions for coupling 1 to PhB(OH)₂ in hand, the versatility of groups that could be installed on the porphyrin framework in this way (FIG. 24) was explored. Because of the inherent congestion of the bis-pocket motif, we were particularly interested in exploring the limitations imposed by the size of the substituent to be installed. We began by increasing the bulk at the 3 and 5 positions of phenylboronic acid.

Analysis of our previously reported crystal structure of 2-2a highlights that the canting of the phenyl rings poised above and below the plane of the porphyrin causes their 3 and 5 positions to point at each other. The 3,5-difluoro,-dichloro, and-dimethyl derivatives of phenylboronic acid were tolerated in the coupling, but further increase in size to 3,5-di-tert-butylphenylboronic acid afforded no product. Unsurprisingly, introduction of steric bulk at the 2 and 6 positions was detrimental and 2,6-dimethylphenylboronic acid afforded no product. The 4 position of phenylboronic acid could tolerate a range of larger substituents; with methyl, n-propyl, or tert-butyl groups at this position, the coupling product could be successfully performed. In the case of the 4-tert-butylphenylboronic acid, the yield was attenuated and the reaction time was increased to 48 h.

The influence of the electronic properties of the substituents on the coupling of phenylboronic acid derivatives was also studied. Using initially optimized conditions, halogen-functionalized aromatic rings, such as 3,5-difluorophenyl and 4-fluorophenyl, could be installed with moderately high yields. A trend whereby an increase in the number/strength of electron-withdrawing groups systematically decreased the yield. Inclusion of 4-trifluoromethylphenyl and 4-nitrophenyl into the panel afforded results consistent with this trend. To obtain even a 5% yield of the 4-nitrophenyl-coupled product, an extended reaction time of 48 h was used, included an extra equivalent of arylboronic acid, and changed the catalyst to Pd(PPh₃)₄. Installation of the electron-donating 4-methoxyphenyl group was similarly inhibited under the standard reaction conditions. Without changing the standard reaction conditions, we were also able to successfully couple 1 to either 2-naphthylboronic acid or cyclopropylboronic acid. The latter example demonstrating that the strategy is not restricted to forming sp²-sp² C—C bonds.

Given the steric and electronic trends established thus far, we were initially surprised by the poor yield for the product in which eight 3,5-dichlorophenyl groups were installed; better yields were obtained with groups that were both larger and smaller as well as with groups that were more or less electron-withdrawing. Although (dppf) PdCl2 is generally not employed in the coupling of aryl chlorides and arylboronic acids, we hypothesized that the decrease in yield stemmed from over-derivatization. That is, additional 3,5-dichlorophenyl groups were coupled to 3,5-dichlorophenyl groups that had already been attached to the porphyrin scaffold. Mass spectrometric analysis of the crude reaction confirmed this hypothesis; prominent signals were observed for over-coupling (m/z =2172.58, 2285.54, 2394.58). We reasoned that this undesired reactivity could be avoided by using a catalyst that would not readily undergo oxidative addition with aryl chlorides.²⁶ To our benefit, we had already established that Pd(PPh₃)₄, which should be even less competent to couple aryl chlorides than (dppf)PdCl₂, worked well for our desired coupling. Indeed, using 1 mol % Pd (PPh₃)₄ as the catalyst afforded the desired product in 70% yield without any further change to the reaction conditions (FIG. 24).

Having demonstrated the general electronic and steric tolerance of this strategy for porphyrin functionalization, its usefulness in preparing porphyrin precursors suitable for further functionalization was further demonstrated. In addition to the 4-nitrophenyl derivative described above, which can be reduced to afford reactive amine units, we were also able to successfully install 4-vinylphenyl groups, which are amenable to further functionalization via alkene metathesis. 4-Trimethylsilylphenyl groups could be installed, although the reaction proceeded more slowly that many of the others. Increasing the reaction time to 48 h and including an extra equivalent of boronic acid allowed the product to be obtained in 47% yield.

Installation of ester groups, which would be able to either undergo transesterification or saponification was studied. Coupling of 1 and 4-ethoxycarbonylphenylboronic acid was unsuccessful using standard conditions. Simple substitution of the solvent for diglyme and inclusion of an extra equivalent of boronic acid provided the desired product in 40% yield. This example highlights that porphyrin functionalization strategy maintains benefits of Pd-catalyzed C-C bond forming reactions: the modular nature of the synthetic protocol allows for rapid and efficient screening of solvent, catalyst, base, temperature, and reaction time to allow for ready incorporation of a given group. As a further example of the flexibility of the method, we highlight that our standard reaction conditions did not permit the coupling of 1 and 1-methylpyrazole-4-boronic acid but that this product formed in 80% yield using the same toluene-to-diglyme solvent substitution described above and an extra equivalent of boronic acid, but no further optimization of reaction conditions.

The above highlights that the synthesis strategy in Example 2 is amenable to preparing bis-pocket porphyrins with a variety of different substituents. The reaction conditions were optimized with PhB (OH)₂ to obtain a general set of conditions that would allow the feasibility of incorporating these groups. For example, although the coupling with 4-methoxyphenylboronic acid was successful using standard reaction conditions, changing the solvent to diglyme increased the yield 5-fold (FIG. 24).

Bis-pocket Porphyrin Architecture. The aryl substituents introduced at the 2 and 6 positions of the meso phenyl groups create pockets above and below the plane of the porphyrin, giving rise to the “bis-pocket” moniker. By varying the nature and positions of the substituents decorating these aryl rings, the pockets can be sculpted. 13 of the free-base TMS-functionalized porphyrin compounds 2-2, in addition to 2-2a were successfully crystallized. The structures confirm, in all cases, the connectivity of the desired products. In many cases, the porphyrin resides on an inversion center, but in no case is the planarity of the entire porphyrin core crystallographically required. Nevertheless, the porphyrins exhibit little distortion from planarity, with the greatest deviation (RMSD=0.091 Å) observed for 2c, R=3,5-dichlorophenyl.

Because the crystals are not isostructural, well-defined relationships between molecular structure and pocket volume are not anticipated. The shapes and volumes of the pockets are impacted significantly by torsion angles, the shallow potential energy profiles of which allow them to be readily deformed by crystal packing forces. The present structures do, however, reveal the variety of pocket shapes and sizes that can be accessed when the substituents are varied. The structures of a number of the compounds (2-2b, 2-2h, 2-2e, 2-2f, 2-2l, 2-2k, 2-2o) feature a pocket on either side of the plane of the porphyrin and both pockets contain solvent molecules. The structure of 2-2i also features two pockets, one above and one below the plane of the porphyrin, but neither contains a solvent molecule. In the structures of 2-2c and 2-2d, one pocket contains a solvent molecule, and the other does not. Interestingly, these are two of the most sterically congested porphyrins prepared in this study. The internal motions that open one pocket sufficiently to accommodate a solvent molecule cause the other pocket to collapse. A similar effect is observed in the structure of 2-2q, in which adjacent molecules interlock across an inversion center with an N-methylpyrazolyl group of each residing in a pocket of the other.

The tilting of the meso terphenyl groups to open up the pocket on one face leads to closing of the pocket on the opposite face.

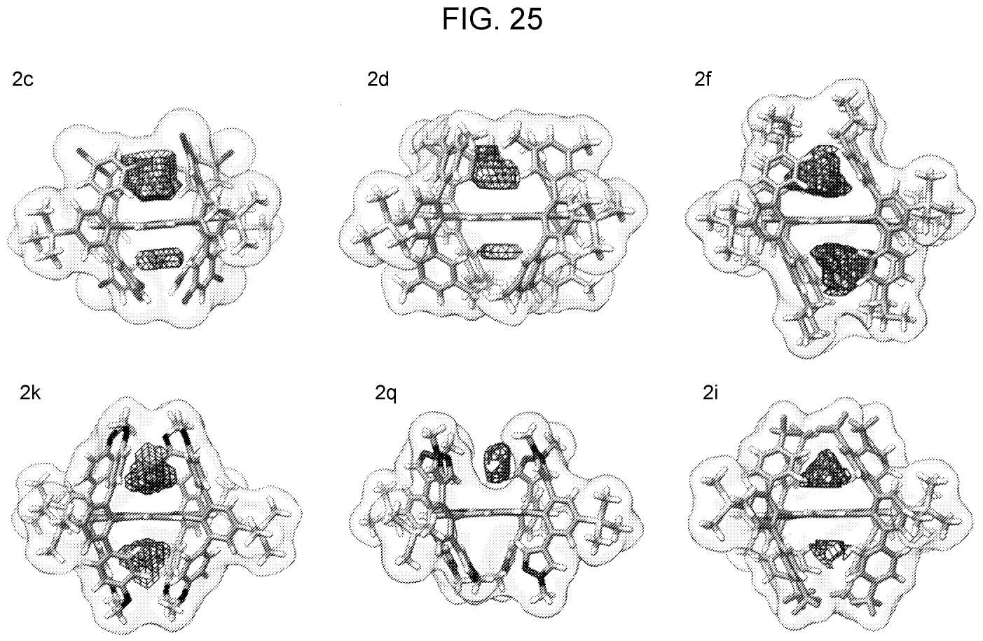

To quantify these variations in pocket volume, we employed POVME2, a tool developed to measure the volumes of protein pockets (FIG. 25, Table 2-8). The absolute values of the volumes obtained from different pocket volume estimation algorithms can vary significantly, but that relative values tend to accurately reflect trends in volumes. Consistent with the analysis above, the volume estimates for 2-2c, 2-2d, and 2-2q reflect the differences in the volumes of the two pockets; whereas 2-2b, 2-2h, 2-2e, 2-2f, 2-2l, 2-2k, 2-2o, and 2-2i features pockets with similar or identical volumes (the latter arising in the case of crystallographic equivalence). The volume estimates also highlight the variation in pocket shape from one molecule to the next. For example, the volumes of the pockets for 2-2a and 2-2l are approximately equal despite the fact that 2-2a features phenyl substituents and 2-2l features the taller naphthyl substituents. The increase in pocket height for 2-2l is offset by a narrowing of the pocket width. The torsionally defined pockets present in these crystal structures are undoubtedly influenced by crystal packing forces in many instances, but we reiterate that they highlight the variability in pocket size/shape that is accessible with this scaffold. This diversity is showcased in FIG. 25.

Sulfonation. The TMS groups present in the porphyrin starting material serve a number of functions. In addition to providing additional ¹H, ¹³C, and ²⁹Si NMR spectroscopic handles, they impart increased organic solubility to 1 facilitating the coupling reaction. The enhanced organic solubility extends to the products, which can be helpful for either solution-phase processing of the products or investigation of their solution-phase properties/reactivity. In addition to advantages related to organic solubility, the TMS groups also provide a means of performing regioselective sulfonation. Sulfonation of these porphyrins can confer upon them greater solubility in polar organic solvents or, in some cases, aqueous solubility. In our previous work, we reported that 5,10,15,20-tetrakis(2,6-diphenyl-4-(trimethylsilyl)phenyl)porphyrinatohydroxoiron (III) could be converted to the corresponding tetrasulfonate salt in 40% yield by treatment with trimethylsilyl chlorosulfonate in refluxing CCl₄ for 1 h, followed by aqueous alkaline work up.²¹ Although we had obtained evidence that this low yield arose from desilylation of the starting material, the 40% yield was sufficient to obtain the amount of material needed to test the CO-sequestering properties of this metalloporphyrin.

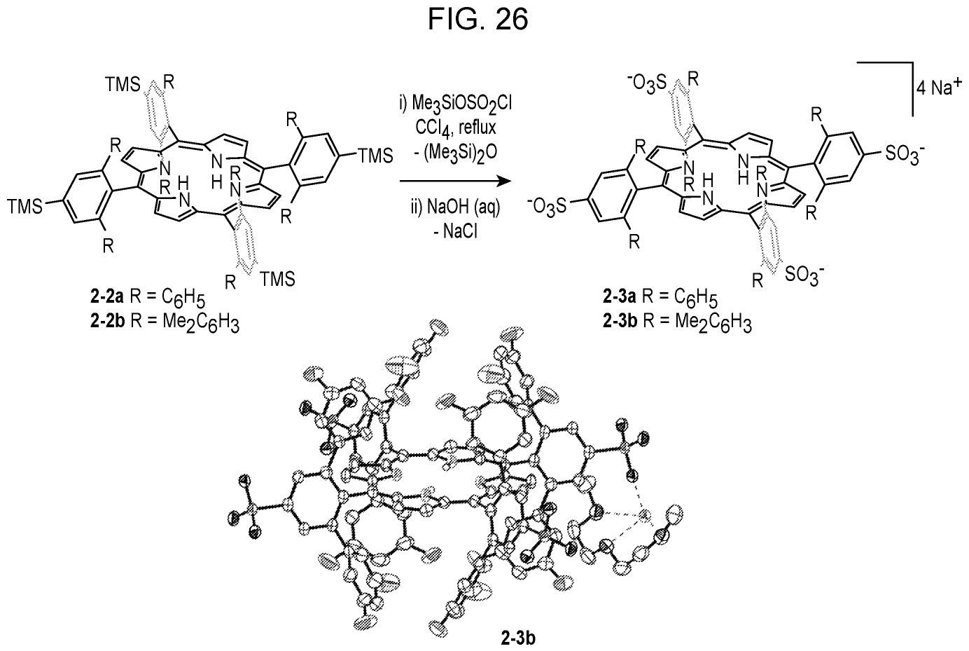

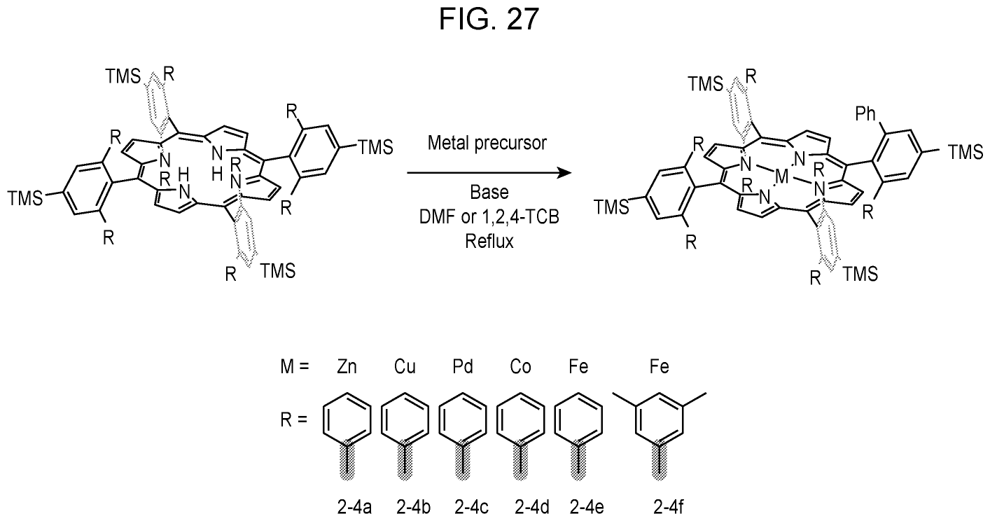

To decrease desilylation, we performed the sulfonation of the free-base 2-2a with fewer equivalents of sulfonating agent (1.2 equiv). The tetrasulfonated product 2-3a was obtained in 60% yield by performing the reaction at 75° C. for 1 h. Compound 2-3b, the tetrasulfonated derivative of 2-2b, was obtained in 84% yield (FIG. 26). This mild reaction provides a convenient means of drastically altering the solubility of the porphyrin: 3a and 3b are readily dissolved in water and methanol, whereas the starting molecules 2a and 2b are completely insoluble in these solvents.

Although all of the porphyrin compounds described generally crystallize well, a number of difficulties in growing diffraction-quality crystals of the porphyrin sulfonate salts were encountered. Crystals grown under a variety of different conditions would exhibit intractable twinning or cracking. Diffraction-quality crystals of 2-3b were grown by including diglyme in the crystallization (FIG. 26). The sodium cation, residing on a general position, is chelated by a molecule of diglyme and otherwise interacts with the sulfonate groups of two symmetry-related porphyrins. The porphyrin itself has 2/m site symmetry (the asymmetric unit contains one quarter of the polyanion), sitting on an inversion center generated by the intersection of a mirror plane and a perpendicular two-fold rotation axis. The porphyrin remains essentially planar (RMSD: 0.029 Å) but the pocket-bounding aryl groups have collapsed to reduce the pocket volume to 7.75 Å³ (from 23.25 Å³ in 2-2b), highlighting the flexibility of the pockets.

Metalation. Although free-base porphyrins can have a variety of valuable properties and reactivities, these molecules are perhaps most widely investigated as ligands for transition metals. The porphyrin scaffold provides a strong thermodynamic preference for metal binding, but the steric bulk of the bis-pocket architecture can provide a significant kinetic barrier to metalation. Although challenging porphyrin metalations are typically performed by heating the free-base ligand with a metal halide and a base in DMF, the original bis-pocket porphyrin report described a process whereby the ligand was heated with Fe(CO)₅ and I₂, followed by aqueous aerobic work up. As shown in Example 1, this approach affords the Fe(III) complex of 2-2a. The Zn(II) and Cu(II) complexes of 2-2a could be readily accessed using standard reaction conditions: refluxing the free-base and excess pyridine in DMF with excess Zn(OAc)₂.2H₂O and CuCl₂.2H₂O, respectively. The ¹H NMR spectrum of the diamagnetic Zn(II) product 2-4a shows the loss of the upfield N-H resonances, as compared to the spectrum of the free ligand, and subtle shifts in the aromatic signals. Additionally, the spectrum features a new singlet of 2H integration at −1.29 ppm. This signal arises from coordination of the Zn center to adventitious water as an aqua ligand. Square-planar coordination is disfavored for Zn(II), providing a strong driving force to coordinate even trace amounts of water. Although coordination to a Lewis acidic metal center would be expected to deshield the protons of the aqua ligand, the geometric position of these protons above the plane of the macrocycle would result in significant shielding from the diamagnetic porphyrin ring current, which is consistent with the upfield location of the resonance (−1.29 ppm). The presence of the aqua ligand was ultimately confirmed crystallographically (FIG. 6) with a Zn—O bond length of 2.194(5) Å. The crystal structure also revealed the Zn center to lie 0.3196(6) Å above the plane of the porphyrin. The porphyrin itself is highly planar with a RMSD of 0.023 Å.

The paramagnetic nature of the Cu(II) complex 2-4b precluded NMR spectroscopic characterization, but single-crystal X-ray diffraction from the red plates of the product confirm insertion of the metal (FIG. 26). The Cu assumes a square-planar geometry with no axial ligand coordination. The primary coordination sphere is rigorously planar, as required by the crystallographic site symmetry of the complex. Beyond the primary coordination sphere of the metal, the porphyrin ligand retains a planar configuration with a RMSD from planarity of 0.018 Å.

Attempts to form the Pd(II) complex in the same way using Pd(OAc)₂ were unsuccessful. Refluxing an excess of Pd(OAc)₂ with ligand 2a in 1,2,4-TCB, however, resulted in insertion into the macrocycle, as indicated by mass spectrometric analysis. We hypothesize that the higher refluxing temperature of 1,2,4-TCB, as compared to DMF, provides the greater activation energy needed to metalate the bulky porphyrin. The mass spectrometric analysis of the reaction mixture revealed, however, that side-products featuring loss of TMS groups formed during the reaction, in addition to the desired product. We confirmed that the temperature of the reaction alone is not sufficient to induce desilylation of the starting porphyrin and therefore suspect that the Pd itself is effecting this transformation. By using only 1 equiv of Pd(OAc)₂, the desilylation was decreased and the Pd(II) complex could be isolated in 25% yield. The complex is diamagnetic and features all of the expected resonances. Unlike the Zn(II) complex, there are no additional features to the spectrum, consistent with the square-planar geometry expected for a Pd(II) complex. Single-crystal X-ray diffraction revealed the Pd(II) complex to be isostructural with the Cu(II) complex.

Refluxing metal halide, lutidine, and 2-2a in 1,2,4-TCB also permitted insertion of Co(II). The reaction proceeds smoothly and the resulting paramagnetic Co(II) complex, 2-4d, was isolated in 91% yield. X-ray crystallography confirmed the formation of a square-planar complex that is isostructural with the Pd(II) and Cu(II) complexes.

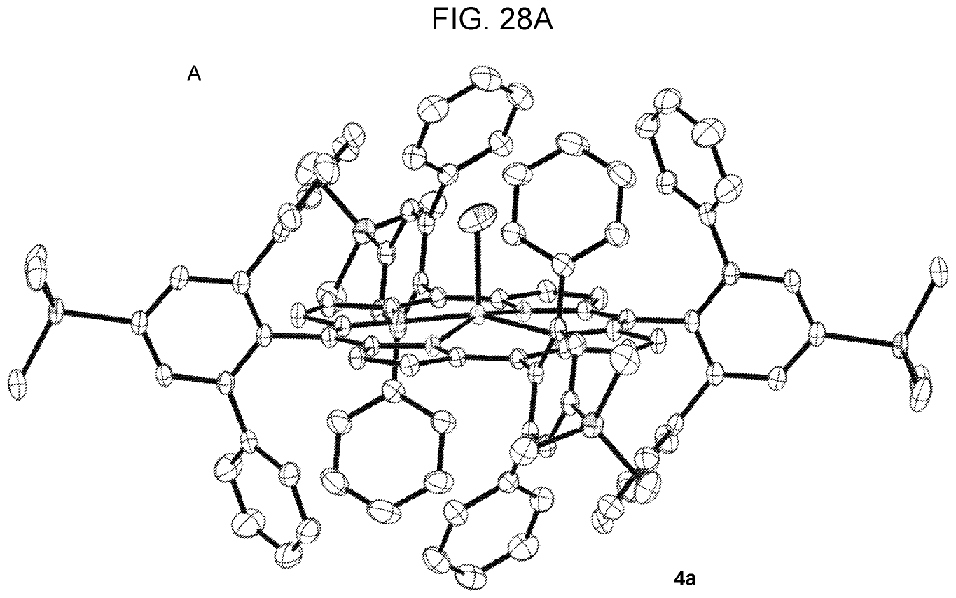

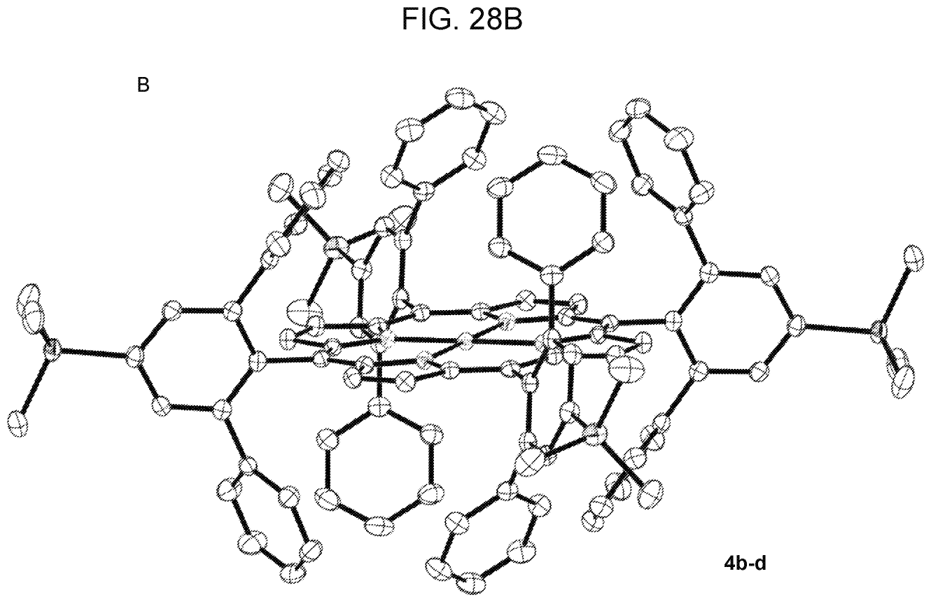

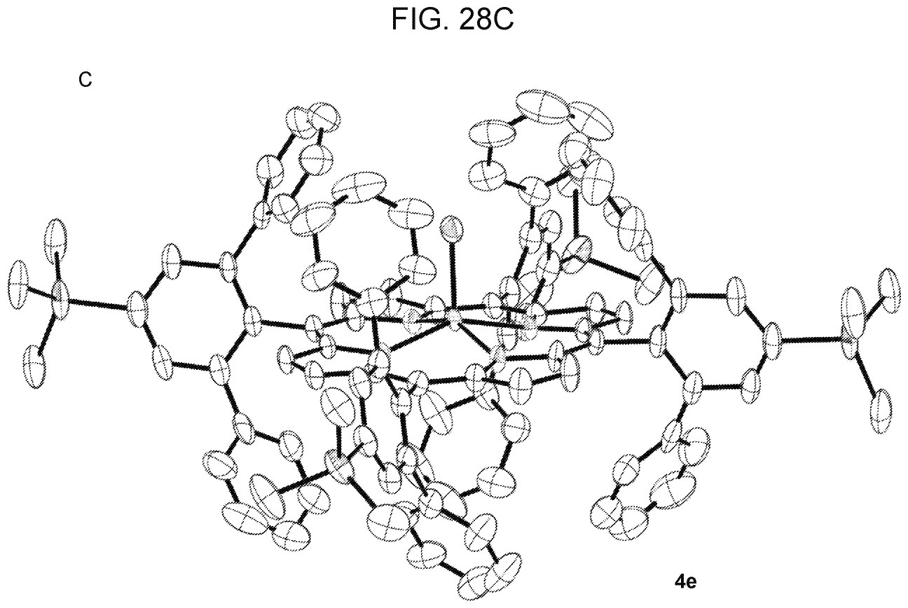

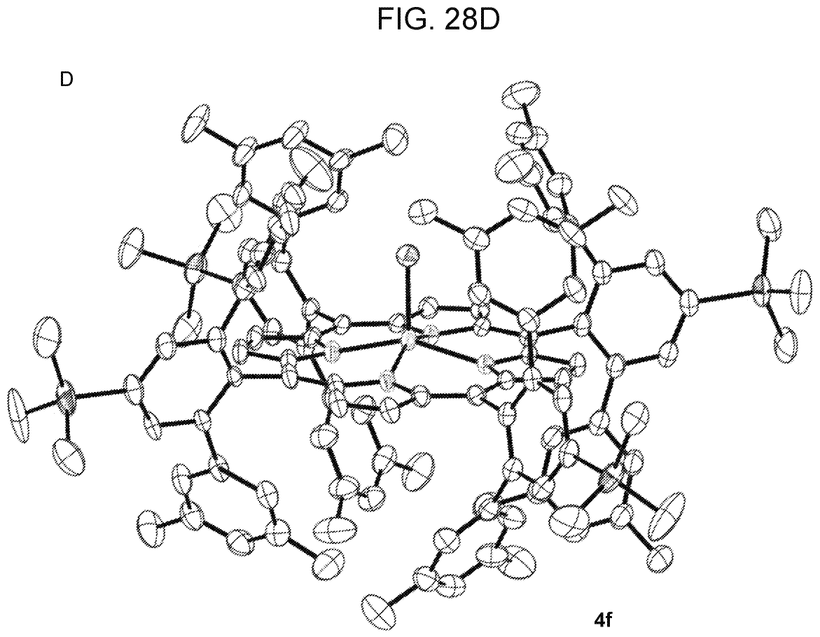

It was also studied whether Fe could be inserted into 2-2a directly using a metal halide as opposed to the circuitous route involving Fe(CO)₅ described above. As observed with Pd, refluxing the porphyrin ligand with an excess of FeCl₂ and lutidine in DMF afforded no product. In contrast, refluxing 1,2,4-TCB cleanly produced 5,10,15,20-tetrakis(2,6-diphenyl-4-(trimethylsilyl)phenyl)porphyrinatochloroiron(III), 2-4e. The ¹H NMR spectrum of this paramagnetic complex clearly shows the characteristic β-pyrrole signal at 80.42 ppm. Single-crystal X-ray diffraction confirms formation of the desired complex (FIG. 28). The Fe center is displaced 0.492(3) Å from the plane of the porphyrin, which adopts a flat configuration (RMSD=0.069 Å). In contrast to the Fe(CO)₅/I₂/alkaline hydrolysis procedure, which affords an Fe(III) hydroxide complex, the presently described reaction affords an Fe(III) chloride complex with an Fe—Cl bond length of 2.203(5) Å. We then demonstrated that this metalation protocol is also capable of producing the Fe(III) chloride derivative of 2-4d, which bears the even more sterically encumbered 2,6-bis(3,5-dimethylphenyl)-4-trimethylsilylphenyl meso substituents. With this ligand, the Fe(CO)₅/I₂/alkaline hydrolysis method was unsuccessful, whereas refluxing 2-2d with excess FeCl₂ and 2,6-lutidine in 1,2,4-TCB affords the product in 71% yield. Again, crystallographic analysis (FIG. 28) reveals that the Fe center is displaced from the plane of the porphyrin (0.4993(15) Å) and that the axial ligand is a chloride (Fe—Cl=2.213(2) Å). Given that two equivalents of Cl⁻ are expected to be eliminated from the reaction as 2,6-lutidinium chloride, the Cl atom at the axial position is believed to come from cannibalized excess FeCl₂.

### CONCLUSIONS

Example 2 shows that Pd-catalyzed Suzuki-Miyaura cross coupling can be readily performed with an easily synthesized free-base porphyrin to access a range of novel porphyrins. This reaction proved versatile in that the steric and electronic properties of the resulting porphyrins could be readily tuned. Substituents featuring a variety of synthetic handles could be installed, rendering the bis-pocket porphyrin products amenable to further modification. The TMS groups of the precursor 1 and the products 2-2 impart organic solubility, which can be readily converted to aqueous solubility upon sulfonation with trimethylsilyl chlorosulfonate. Finally, metalation could be readily achieved using standard protocols for some metals, or refluxing 1,2,4-TCB when necessary.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112 (f) or 35 U.S.C. § 112 (6) is expressly defined as being invoked for a feature in the claim only when the exact phrase “means for” or the exact phrase “step for” is recited at the beginning of such feature in the claim; if such exact phrase is not used in a feature in the claim, then 35 U.S.C. § 112 (f) or 35 U.S.C. § 112 (6) is not invoked.

What is claimed is:

1. A compound of formula I: 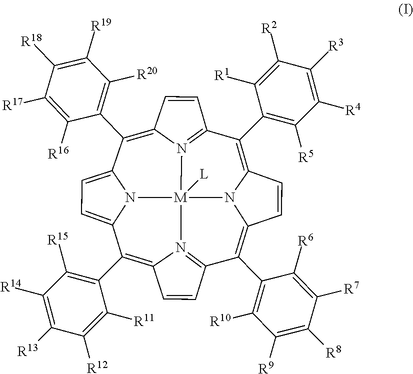
wherein:
R₂, R₄, R₇, R₉, R₁₂, R₁₄, R₁₇ and R₁₉ are each independently selected from hydrogen, hydroxy, alkoxy, amine, cyano, thiol, halogen, alkyl, substituted alkyl, haloalkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
R₁, R₅, R₆, R₁₀, R₁, R₁₅, R₁₆ and R₂₀ are each independently selected from substituted alkyl, haloalkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
R₃, R₈, R₁₃ and R₁₈ are each independently a water soluble group;
M is a metal; and
L is a ligand, or a salt, solvate or hydrate thereof.

2. The compound according to claim 1, wherein M is Fe.

3. The compound according to claim 1 wherein R₂, R₄, R₇, R₉, R₁₂, R₁₄, R₁₇ and R₁₉ are each hydrogen.

4. The compound according to any one of claims 1-3, wherein R₁, R₅, R₆, R₁₀, R₁₁, R₁₅, R 16 and R₂₀ are each selected from aryl, substituted aryl, arylalkyl, substituted arylalkyl, heteroaryl, substituted heteroaryl, heteroarylalkyl, and substituted heteroarylalkyl.

5. The compound according to claim 4, wherein R₁, R₅, R₆, R₁₀, R₁₁, R₁₅, R₁₆ and R₂₀ are each an aryl group.

6. The compound according to claim 5, wherein R₁, R₅, R₆, R₁₀, R₁₁, R₁₅, R₁₆ and R₂₀ are each independently selected from the group consisting of phenyl, 4-methylphenyl, 4-propylphenyl, 4-tertbutylphenyl, 3,5-dimethylphenyl, and 3,5-dichlorophenyl.

7. The compound according to claim 6, wherein R₁, R₅, R₆, R₁₀, R₁₁, R₁₅, R₁₆ and R₂₀ are each independently selected from the group consisting of:

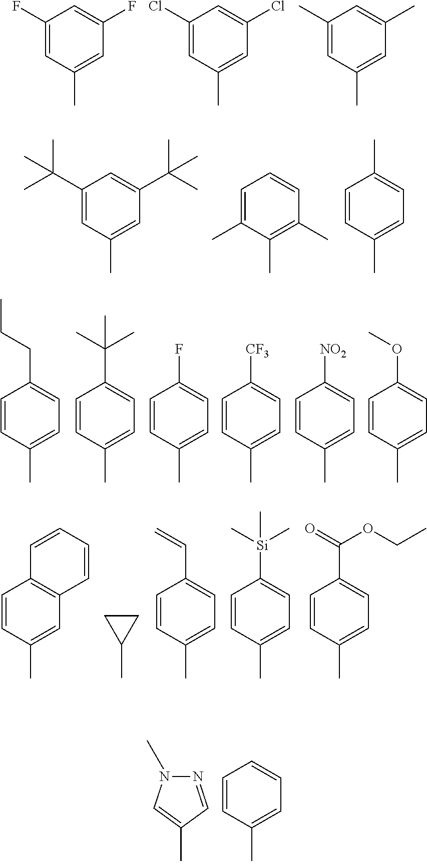

8. The compound according to any one of claims 1-7, wherein R₃, R₈, R₁₃ and R₁₈ are each independently the water soluble group is selected from trimethylsilyl, sulfonate, carboxylate, ammonium, trialkylammonium, pyridinium, N-alkylpyridinium, or poly (ethylene glycol).

9. The compound according to any one of claims 1-8, wherein L is hydroxy.

10. The compound according to claim any one of claims 1-9, wherein the compound is sodium 5,10,15,20-tetrakis(2,6-diphenyl-4-(sulfonate)phenyl)porphyrinatohydroxoiron(III):

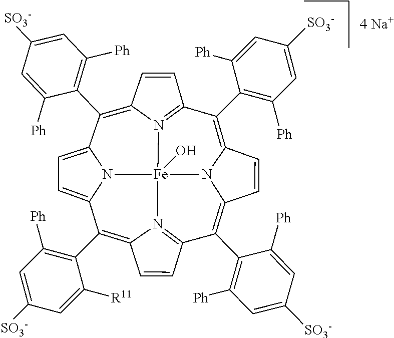

11. The compound according to any one of claims 1-10, wherein the compound is formed from contacting the sodium 5,10,15,20-tetrakis(2,6-diphenyl-4-(sulfonate)phenyl)porphyrinatohydroxoiron(III) with a reducing agent selected from the group consisting of alkylthiols, substituted alkylthiols, arylthiols, substituted arylthiols, thiol-bearing amino acids and thiol-bearing peptides.

12. The compound according to any one of claims 1-11, wherein the compound is selected from:
5,10,15,20-tetrakis(2,6-diphenyl-4-(trimethylsilyl)phenyl)porphyrin;
5,10,15,20-tetrakis(2,6-di(3,5-difluoro)phenyl-4-(trimethylsilyl)phenyl)porphyrin;
5,10,15,20-tetrakis(2,6-di(3,5-dichloro)phenyl-4-(trimethylsilyl)phenyl)porphyrin;
5,10,15,20-tetrakis(2,6-di(3,5-dimethyl)phenyl-4-(trimethylsilyl)phenyl)porphyrin;
5,10,15,20-tetrakis(2,6-di(4-methyl)phenyl-4-(trimethylsilyl)phenyl)porphyrin;
5,10,15,20-tetrakis(2,6-di(4-n-propyl)phenyl-4-(trimethylsilyl)phenyl)porphyrin;
5,10,15,20-tetrakis(2,6-di(4-tert-butyl)phenyl-4-(trimethylsilyl)phenyl)porphyrin;
5,10,15,20-tetrakis(2,6-di(4-fluoro)phenyl-4-(trimethylsilyl)phenyl)porphyrin;
5,10,15,20-tetrakis(2,6-di(4-trifluoromethyl)phenyl-4-(trimethylsilyl)phenyl)porphyrin;
5,10,15,20-tetrakis(2,6-di(4-nitro)phenyl-4-(trimethylsilyl)phenyl)porphyrin;
5,10,15,20-tetrakis(2,6-di(4-methoxy)phenyl-4-(trimethylsilyl)phenyl)porphyrin;
5,10,15,20-tetrakis(2,6-di(2-napthyl)phenyl-4-(trimethylsilyl)phenyl)porphyrin;
5,10,15,20-tetrakis(2,6-dicyclopropyl-4-(trimethylsilyl)phenyl)porphyrin;
5,10,15,20-tetrakis(2,6-di(4-vinyl)phenyl-4-(trimethylsilyl)phenyl)porphyrin;
5,10,15,20-tetrakis(2,6-di(4-trimethylsilyl)phenyl-4-(trimethylsilyl)phenyl)porphyrin;
5,10,15,20-tetrakis(2,6-di(4-ethoxycarbonyl)phenyl-4-(trimethylsilyl)phenyl)porphyrin;
5,10,15,20-tetrakis(2,6-di(N-methylpyrrazolyl)phenyl-4-(trimethylsilyl)phenyl)porphyrin;
5,10,15,20-tetrakis(2,6-diphenyl-4-(sulfonato)phenyl)porphyrin;
5,10,15,20-tetrakis(2,6-di(3,5-difluoro)phenyl-4-(sulfonato)phenyl)porphyrin;
5,10,15,20-tetrakis(2,6-diphenyl-4-(trimethylsilyl)phenyl)porphyrinatoaquazinc(II);
5,10,15,20-tetrakis(2,6-diphenyl-4-(trimethylsilyl)phenyl)porphyrinatocopper(II);
5,10,15,20-tetrakis(2,6-diphenyl-4-(trimethylsilyl)phenyl)porphyrinatopalladium(II);
5,10,15,20-tetrakis(2,6-diphenyl-4-(trimethylsilyl)phenyl)porphyrinatocobalt(II);
5,10,15,20-tetrakis(2,6-diphenyl-4-(trimethylsilyl)phenyl)porphyrinatochloroiron(III); and
5,10,15,20-tetrakis(2,6-di(3,5-dimethyl)phenyl-4-(trimethylsilyl)phenyl)porphyrinatochloroiron(III)

13. A composition comprising:
a compound according to any one of claims 1-12; and
a pharmaceutically acceptable excipient.

14. A method for sequestering carbon monoxide, the method comprising contacting a composition comprising carbon monoxide with a compound according to any one of claims 1-12.

15. A method for treating a subject exposed to carbon monoxide, the method comprising administering to the subject a therapeutically effective amount of a compound according to any one of claims 1-12.
